SEQUENCE LISTING (38 sequences)

SEQ ID NO: 1 - DNA, 22 bp, Artificial Sequence
  Bcl11b-cko-FW Genotyping PCR primer
tgagtcaataaacctgggcgac

SEQ ID NO: 2 - DNA, 23 bp, Artificial Sequence
  Bcl11b-cko-RV Genotyping PCR primer
ggaatccttggagtcacttgtgc

SEQ ID NO: 3 - DNA, 20 bp, Artificial Sequence
  Bcl11b-cko-DEL Genotyping PCR primer
tcctggtaacacacaattgc

SEQ ID NO: 4 - DNA, 19 bp, Artificial Sequence
  Notch1-Fwd qPCR primer
cccttgctctgcctaacgc

SEQ ID NO: 5 - DNA, 19 bp, Artificial Sequence
  Notch1-Rev qPCR primer
ggagtcctggcatcgttgg

SEQ ID NO: 6 - DNA, 21 bp, Artificial Sequence
  Ets1-Fwd qPCR primer
ttaggaaaggctcgtttgctc

SEQ ID NO: 7 - DNA, 22 bp, Artificial Sequence
  Ets1-Rev qPCR primer
ccaaagcacaagcatagtttgc

SEQ ID NO: 8 - DNA, 19 bp, Artificial Sequence
  Hes1-Fwd qPCR primer
ccagccagtgtcaacacga

SEQ ID NO: 9 - DNA, 21 bp, Artificial Sequence
  Hes1-Rev qPCR primer
aatgccgggagctatctttct

SEQ ID NO: 10 - DNA, 21 bp, Artificial Sequence
  Gata3-Fwd qPCR primer
ctcggccattcgtacatggaa

SEQ ID NO: 11 - DNA, 20 bp, Artificial Sequence
  Gata3-Rev qPCR primer
ggatacctctgcaccgtagc

SEQ ID NO: 12 - DNA, 23 bp, Artificial Sequence
  Deltax1-Fwd qPCR primer
tgttcaggctatacacgcatcaa

SEQ ID NO: 13 - DNA, 18 bp, Artificial Sequence
  Deltax1-Rev qPCR primer
ccaccgcccactttcaag

SEQ ID NO: 14 - DNA, 20 bp, Artificial Sequence
  Tcf1-Fwd qPCR primer
atgggcggcaactctttgat

SEQ ID NO: 15 - DNA, 19 bp, Artificial Sequence
  Tcf1-RevqPCR primer
cgtagccgggctgattcat

SEQ ID NO: 16 - DNA, 20 bp, Artificial Sequence
  Cdkn1c-Fwd qPCR primer
cgaggagcaggacgagaatc

SEQ ID NO: 17 - DNA, 21 bp, Artificial Sequence
  Cdkn1c-Rev qPCR primer
gaagaagtcgttcgcattggc

SEQ ID NO: 18 - DNA, 21 bp, Artificial Sequence
  Id2-Fwd qPCR primer
atgaaagccttcagtccggtg

SEQ ID NO: 19 - DNA, 19 bp, Artificial Sequence
  Id2-Rev qPCR primer
agcagactcatcgggtcgt

SEQ ID NO: 20 - DNA, 19 bp, Artificial Sequence
  Il2rb-Fwd qPCR primer
tggagcctgtccctctacg

SEQ ID NO: 21 - DNA, 21 bp, Artificial Sequence
  Il2rb-Rev qPCR primer
tccacatgcaagagacattgg

SEQ ID NO: 22 - DNA, 21 bp, Artificial Sequence
  Zfp105-Fwd qPCR primer
ggcatccagccaacaagtgta

SEQ ID NO: 23 - DNA, 23 bp, Artificial Sequence
  Zfp105-Rev qPCR primer
catttcctgacccttttcctcat

SEQ ID NO: 24 - DNA, 20 bp, Artificial Sequence
  Traf1-Fwd qPCR primer
ggaggcatcctttgatggta

SEQ ID NO: 25 - DNA, 20 bp, Artificial Sequence
  Traf1-Rev qPCR primer
agggacaggtgggtcttctt

SEQ ID NO: 26 - DNA, 21 bp, Artificial Sequence
  Zbtb32-Fwd
gctctgagagaggacttggga

SEQ ID NO: 27 - DNA, 23 bp, Artificial Sequence
  Zbtb32-Rev qPCR primer
tgctttatgcttgtgtgacatct

SEQ ID NO: 28 - DNA, 24 bp, Artificial Sequence
  TCRB_Dbeta2-Fwd PCR primer
gtaggcacctgtggggaagaaact

SEQ ID NO: 29 - DNA, 20 bp, Artificial Sequence
  TCRB_Vbeta2-Fwd PCR primer
gggtcactgatacggagctg

SEQ ID NO: 30 - DNA, 25 bp, Artificial Sequence
  TCRB_Jbeta2-Rev PCR primer
tgagagctgtctcctactatcgatt

SEQ ID NO: 31 - DNA, 22 bp, Artificial Sequence
  BS1-Fwd qPCR pimer for ChIP assay
ccgctacgaggcaccctccttt

SEQ ID NO: 32 - DNA, 24 bp, Artificial Sequence
  BS1-Rev qPCR primer for ChIP assay
agtctccttgggaagcacgcgcta

SEQ ID NO: 33 - DNA, 29 bp, Artificial Sequence
  BS2-Fwd qPCR primer for ChIP assay
gcttgcttgtttttaattcagtttatggg

SEQ ID NO: 34 - DNA, 28 bp, Artificial Sequence
  BS2-Rev qPCR primer for ChIP assay
ttgaatgtctgtgttggtgtgtaatcac

SEQ ID NO: 35 - DNA, 24 bp, Artificial Sequence
  BS3-Fwd qPCR primer for ChIP assay
gtgaaaaaaagggggtaggccctc

SEQ ID NO: 36 - DNA, 25 bp, Artificial Sequence
  BS3-Rev qPCR primer for ChIP assay
cagcccaaagtcaaaaggcaagatg

SEQ ID NO: 37 - DNA, 27 bp, Artificial Sequence
  CTL-Fwd qPCR primer for ChIP assay
gttccttaactgagagttcctcctccc

SEQ ID NO: 38 - DNA, 22 bp, Artificial Sequence
  CTL-Rev qPCR primer for ChIP assay
tcactctgggccggagtcagtt

(19) United States
(12) Patent Application Publication
(10) Pub. No.: US 2012/0288484 A1
(43) Pub. Date: Nov. 15, 2012

(54) CELLS, COMPOSITIONS AND METHODS

(75) Inventors: Pentao Liu, London (GB); Peng Li, London (GB); Shannon Burke, London (GB)

(73) Assignee: Genome Research Limited, London (GB)

(21) Appl. No.: 13/384,081

(22) PCT Filed: Jul. 15, 2010
(86) PCT No.: PCT/GB2010/051158

§ 371 Date: Jul. 30, 2012

Related U.S. Application Data
(60) Provisional application No. 61/225,779, filed on Jul. 15, 2009.

(30) Foreign Application Priority Data
Jul. 15, 2009 (GB) 0912287.0
Apr. 21, 2010 (GB) 1006649.6

(51) Int. Cl. C12N 5/0783; A61P 35/00; A61P 31/12; A61K 35/12

(52) U.S. Cl. 424/93.71; 435/375; 435/325

(57) ABSTRACT

Method of producing induced T-to-Natural-Killer [ITNK] cells, target T cells and/or target pro-T cells from T cells and/or pro-T cells which method involves modulating the activity and/or effect of at least one Bcl11b gene and/or protein present in a T cell and/or pro-T cell, and converting said T cell and/or pro-T cell to an ITNK cell or target Tcells and/or target pro-T cells is described. ITNK cells, target T cells and/or target pro-T cells produced by such method and mature activated T cells in which Bcl11b expression is downregulated or absent, and the use of such cells or modulators of Bcl11b in medicine is also described.

The present invention relates to induced T-to-Natural-Killer cells [herein “ITNK” cells], methods for their production and use of such cells, as well as methods for producing T cells.

Natural killer (NK) cells are a type of cytotoxic lymphocyte that constitute a major component of the innate immune system. NK cells play a major role in the rejection of tumors and cells infected by viruses and microbes. NK-cells are large granular lymphocytes (LGL) and constitute cells differentiated from stem cells or multipotent progenitors. The molecular mechanisms controlling the development of different cell types from stem cells is not fully understood.

### STATEMENTS OF INVENTION

The invention provides a method of producing induced T-to-Natural-Killer [ITNK] cells from T cells and/or pro-T cells, the method comprising modulating the activity and/or effect of the Bcl11b gene and/or Bcl11b protein present in a T cell or pro-T cell, thereby converting said T cell and/or pro-T cell to an ITNK cell.

The invention provides a method of producing target T cells and/or target pro-T cells, the method comprising modulating the activity and/or effect of at least one Bcl11b gene and/or protein product present in a T cell and/or pro-T cell, and converting said T cell and/or pro-T cell to said target T cells and/or target pro-T cells.

The invention provides an ITNK cell obtainable, or obtained, from a T cell or pro-T cell. Suitably the T cell or pro-T cell includes a Bcl11b gene and/or gene product the activity and/or effect of which has been modulated so that the T cell or pro-T cell is capable of conversion to a ITNK cell.

The invention also relates to mature activated T cells in which Bcl11b expression is downregulated or absent (hereafter referred to as TBcl11b-cells), for use in medicine, such as prophylaxis or treatment of disease. The invention also relates to isolated or purified mature activated T cells in which Bcl11b expression is downregulated or absent.

The invention provides a target T cell or target pro-T cell obtainable, or obtained, from a T cell or pro-T cell respectively. Suitably the target cell comprises at least one Bcl11b gene and/or gene product the activity and/or effect of which has been modulated when compared to the wild type cell, so that the target T cell or target pro-T cell is capable of conversion to an ITNK cell. Wild type cells in the context of this disclosure does not refer to cancerous or transformed cells.

The invention provides a pharmaceutical composition comprising ITNK cells, or target T cells, or target pro-T cells together with a pharmaceutically acceptable excipient.

The invention provides ITNK cells or target T cells or target pro-T cells for use in therapy.

The invention provides a method of treating a human or non-human mammal subject suffering from, or susceptible to disease such as cancer or viral infection, the method comprising administering to the subject a therapeutically effective amount of ITNK cells or target T cells/pro-T cells, preferably ITNK cells or target T cells/pro-T cells which are derived from T cells or pro-T cells that have been obtained from that subject.

The invention provides a method of treating a human or non-human mammal subject suffering from, or susceptible to disease such as cancer or viral infection, the method comprising administering to the subject a therapeutically effective amount of a compound which modulates or inhibits the expression, activity and/or effect of Bcl11b gene or protein in T cells or pro-T cells and leads to the conversion of these T cells or pro-T cells to ITNK cells.

The invention provides an assay for identifying a target with which the Bcl11b gene product and/or protein product interacts or has an effect thereon, which assay comprises modulating the activity of a Bcl11b gene and/or gene product and monitoring the interaction or effect on a potential downstream target. Optionally a downstream target thus identified is modified to cause or assist in ITNK cell production.

The invention also relates to upstream modulators of Bcl11b activity, suitably those capable of causing or assisting in the conversion of T cells or pro-T cells to ITNK cells or target T cells/pro T cells. The invention also relates to methods for identification of upstream modulators of Bcl11b comprising identification of compounds that are able affect Bcl11b gene or protein expression or activity or effect, suitably as assessed by an effect of the upstream modulator on ITNK formation as disclosed herein.

In one aspect the invention relates to the use of factors which regulate the Bcl11b gene or protein expression or activity, or which are functionally downstream of the Bcl11b gene or protein, or which are functionally upstream of the Bcl11b gene, to effect the conversion of T cells to ITNK cells, and to the use of modulators of these factors to effect the conversion of T cells to ITNK cells.

The invention provides an assay for identification of a compound which assists in the reprogramming of T cells or pro-T cells to ITNK cells, the method comprising contacting T cells or pro-T cells with a test compound and monitoring or selecting for the conversion of T cells to ITNK cells or target T/pro T cells.

The invention provides an assay for identification of a mutation which results in or contributes to the reprogramming of T cells or pro-T cells to ITNK cells, the method comprising mutagenesis of T cells or pro-T cells and monitoring or selecting for the conversion of T cells to ITNK cells, followed by identification of the location of the mutation.

The invention provides an assay for identification of a compound which assists in the reprogramming of T cells to ITNK cells, the method comprising screening for compounds that bind to the Bcl11b DNA or RNA or the Bcl11b protein, and assessing whether said compounds are able to promote the conversion of T cells to ITNK cells.

The invention further provides use of compounds so discovered in the conversion of T cells or pro-T cells to ITNK cells.

The invention further provides a non-human animal carrying ITNK cells, and/or target T cells or target pro-T cells.

### FIGURES AND TABLES

FIG. 1. Bcl11b is essential for T cell development and for maintaining T cell identity.

(A) Flow cytometry profiles of cultured DN1 and DN2 thymocytes (+OHT) in the absence of IL-2.
(B) Flow cytometry profiles of cultured flox/flox DN3 thymocytes (±OHT) supplemented with IL-2.
(C) Killing of OP9-DLI stromal cells by OHT-treated flox/flox DN3 thymocytes.
(D) DNA from purified NKp46⁺ cells was prepared and subjected to PCR to detect DJ (top) and VDJ (bottom) recombination at the TCRβlocus.
(E-G) Microarray analysis of gene expression in NKp46⁺CD3⁺ ITNK cells from DN3 thymocytes.
(E) Two-way hierarchical cluster map of the array data.
(F) and (G) qRT-PCR validation of gene expression of selected genes among ITNKs, LAKs and DN3 cells.

FIG. 2. Efficient reprogramming of T cells to ITNKs.

(A) Representative flow cytometry profiles of ITNKs reprogrammed from single flox/flox DN3 cells.
(B) PCR genotyping of Bcl11b deletion in two representative T cell (T1, T2) and ITNK (I1, I2) wells.
(C) DJ recombination at the TCRβ locus of five ITNK wells (I1-I5) showing unique DJ recombination.
(D) Giemsa stain of parental DN3 thymocytes (T) and ITNK cells.
(E) and (e) Transmission electron micrographs of an ITNK cell.
(F) Cytotoxicity of ITNKs (labeled as “+OHT”) and LAKs measured in standard ⁵¹Cr release assays with B16F10, RMA and RMA-S tumor cell targets at the indicated effector-to-target (E:T) ratios. −OHT: flox/flox T cells.

FIG. 3. ITNKs reprogrammed in vivo were potent tumour cell killers.

(A) Flow cytometric analysis of thymocytes and splenocytes from OHT treated flox/flox and flox/+ mice.
(B) Analysis of ITNKs from thymic γδ T cells in OHT treated flox/flox mice.
(C) ITNKs production in Rag2^{-/-}Il2rg^{-/-} recipients injected with flox/flox DP thymocytes.
(D) Ex vivo expansion of ITNKs in IL-2 from splenocytes of the recipient mice.
(d) Ex vivo expansion of in vivo reprogrammed iTNK cells starting from splenotypes of four Rag2^{-/-}Il2γc^{-/-} recipient mice.
(E) The ex vivo-expanded ITNKs (labeled as “+OHT”) were used in ⁵¹Cr release killing assays with B16F10, RMA and RMA-S tumor cell targets at the indicated effector-to-target (E:T) ratios. −OHT: flox/flox T cells.
(F) ITNKs prevented tumour metastasis. Rag2^{-/-}Il2rg^{-/-} recipients transplanted with treated (+OHT) or untreated (−OHT) flox/flox DP thymocytes or PBS and subsequently injected intravenously with 50,000 B16F10 melanoma cells.
(G) In vivo iTNKs effectively eliminated B16F10 melanoma cells in mice.

FIG. 4. Bcl11b is a direct downstream target gene of Notch signaling.

(A). Bcl11b protein in T cells following OHT treatment detected by Western blot.
(B) Schematic of the Bcl11b locus showing putative CSL binding sites (BS) and that of an irrelevant control binding site (CTL).
(C) Genomic DNA was prepared from immunoprecipitation of thymocytes, using CSL or control IgG antibodies, and was amplified using primers flanking the putative CSL or the control binding sites at the Bcl11b locus.

FIG. 5. Generation of the Bcl11b-tdTomato reporter mouse.

(A) The tdTomato cassette was targeted to the 3′ UTR of the Bcl11b locus.
(B) Insertion of the tdTomato cassette at the Bcl11b 3′ UTR did not affect T cell development.

FIG. 6. Detection of Bcl11b expression in hematopoietic lineages using the Bcl11b-tdTomato reporter mice.

(A) CD4 CD8 double negative (DN; DN1-DN4) thymocyte subsets.
(B) Double positive (DP) thymocytes (CD4⁺CD8⁺), splenic CD4⁺ and CD8⁺ T cells, thymic γδ T cells, and splenic NKT cells (CD3⁺CD1d⁺).
(C) Bone marrow B cells (CD19⁺B220⁺) and myeloid cells (CD11b⁺Gr-1⁺).
(D) Splenic (CD3), and thymic (CD3CD4CD8) NK cells.
(E) qRT-PCR of Bcl11b expression in sorted splenic naïve (CD44⁻CD62L⁺) and activated (CD44⁺CD62L⁻) T cells population.
(F) Quantification of Bcl11b expression in naïve and activated T cells in the Bcl11b^{td/+} mice.

FIG. 7. Strategies for identification of cell populations for flow sorting and analysis.

(A) Identification of double negative (DN) thymocyte (DN1-DN4) populations defined by Lin and expression of CD25 and CD44.
(B) Identification of γδ T cells.
(C) Identification of NKT cells in the spleen by first gating (or, prior to FACS sorting, magnetically depleting) out B cells.
(D) Identification of NK precursors and NK cell subsets cells.
(E) Thymic NK cells were defined as NK1.1⁺CD127⁺ thymocytes.
(F) Identification of naïve (CD44⁻CD62L⁺) and activated (CD44⁺CD62L⁻) T cells.

FIG. 8. In vitro analysis of Bcl11b-deficient T cells.

(A) Schematic diagram of the Bcl11b conditional knockout allele. (B) Experimental design for the analysis of Bcl11b-deficient DN thymocytes. (C) NKp46⁺CD3 cells from DN1 and DN20HT-treated flox/flox thymocytes did not express TCRβ.
(D) Homozygous Bcl11b deletion in ITNK (NKp46⁺CD3) but not in T (NKp46⁻CD3⁺) cell populations from DN1 and DN2 cultures.
(E) No NKp46⁺ cells but T cells were obtained from untreated flox/flox thymocytes.
(F) NKp46⁺TCRβ⁻ cells from OHT-treated DN1 and DN2 flox/flox thymocytes in the absence of IL-2 or IL-15 cultured on OP9 stromal cells.
(G) NKp46⁺TCRβ⁻ cells were detected in OHT-treated DN3 flox/flox, but not flox/+, thymocytes in T cell media.
(H) Reprogramming of Bcl11b-deficient DN3 thymocytes to NKp46⁺ cells in myeloid cell culture condition.
(I) Reprogramming of Bcl11b-deficient DN3 thymocytes to NKp46⁺CD19⁻ cells in B cell culture condition.
(J) Venn diagram comparison of the upregulated (>2-fold) genes between LAK vs DN3 (green) and ITNK vs DN3 (purple).
(K) ITNKs from DP flox/flox thymocytes treated with OHT and cultured on OP9-DL1 in the presence of IL-2.
(L) ITNKs from splenic flox/flox CD8⁺ T cells treated with OHT cultured on OP9-DL1 in the presence of IL-2.

FIG. 9. Characterization of in vitro reprogrammed ITNK phenotype.

(A) and (a) Experimental design for reprogramming of single DN3 thymocytes to ITNK.
(B-C) Expression of intracellular and NK cell surface markers by the reprogrammed ITNK from DN3 thymocytes in vitro.
(D) Expression of NK cell markers by ITNKs reprogrammed from Bcl11b-deficient DP thymocytes in vitro.
(E) ITNKs did not express CD127 and thus were not thymic NK cells.
(F) Analysis of CD27 and CD11b in bulk-cultured ITNKs reprogrammed from DN3 thymocytes.

FIG. 10. Analysis of in vivo reprogrammed ITNK cells in the flox/flox mouse.

(A) Experimental design for the analysis of in vivo reprogrammed ITNK cells.
(B) PCR of Bcl11b deletion in ITNK (NKp46⁺CD3⁺ and NKp46⁺CD3⁻) cell populations in flox/flox mice.
(C) Flow cytometric analysis of CD4 and CD8 expression in NKp46⁺ ITNKs.
(D) Flow cytometric analysis of cells following ex vivo expansion of whole thymocytes or splenocytes from OHT treated mice.
(E) Flow cytometric analysis of CD1d-restriced NKT cells in thymus and spleen.
(F) Analysis of CD1d-restricted cells in the ex vivo-expanded ITNK culture.
(G) qRT-PCR analysis of several key T or NK cell-associated genes in CD8⁺ T cells, CD8⁺ ITNKs and LAKs.
(H) Splenocytes from flox/flox or flox/+ mice treated with Tamoxifen were stained with NKp46, NK1.1, CD8 and CD3 to confirm expression of CD3 on ITNKs.

FIG. 11. In vivo reprogrammed ITNKs from DP thymocytes prevented tumour metastasis.

(A) Experimental design for the analysis of in vivo reprogramming of DP thymocytes to ITNKs.
(B) Most ITNKs in the spleen were CD8⁺.
(C) ITNKs had complete Bcl11b deletion whereas donor derived NKp46 cells still retained at least one copy of the foxed allele. (D) ITNKs were also found in bone marrow and peripheral blood.
(E) Expression of additional NK cell surface markers on the in vivo reprogrammed ITNKs.
(F) ITNKs prevented tumour metastasis. Rag2^{-/-}Il2rg^{-/-} recipients were transplanted with treated (+OHT) or untreated (−OHT) flox/flox DP thymocytes or PBSand subsequently injected intravenously with 5×10⁴B16F10 melanoma cells.
(G) Plot shows inverse correlation between the percentage of ITNK cells (squares) obtained from recipient mice following in vivo reprogramming and tumor challenge and the number of lung colonies (circles) observed.

FIG. 12. A working model showing that Bcl11b acts downstream of Notch signaling and promotes T cell development and maintains T cell identity.

FIG. 13.
a. Expression of Bcl11b in thymocytes from Bcl11b-lacZ knock-in mice using the fluorescent substrate FDG.
b. Detection of Bcl11b expression in the five DN1 subpopulations.
c. Top, acute loss of Bcl11b caused DN1 thymocytes to express NK-specific genes. Bottom, deleting Bcl11b in DN2 thymocytes gave rise to the same phenotype of converting to NK-like cells.

FIG. 14.
a. Left panel: different double negative (DN) thymocyte populations; Right panel: five subpopulations of DN1 thymocytes.
b. Flow chart of analyzing Bcl11b-deficient DN1 thymocytes.

FIG. 15.
a. Double positive (DP) thymocytes expressed NKp46 after Bcl11b deletion.
b. Purified CD8 single positive cells (−OHT) proliferated on OP9-DL1 stromal cells. They did not express NKp46. Once Bcl11b was deleted, 38% of the cells now expressed NKp46 which killed the stromal cells.
c. Purified CD4 single positive cells (−OHT) growing in T cell media (left). Bcl11b deletion (+OHT) caused these CD4 T cells to express NKp46 and NKG2D.
   Table 1. Comparison of gene expression profiles of ITNK, DN3 and LAK cells in microarray analysis.
   Table 2. Comparison of cell surface receptor repertoire of LAK and ITNKs.
   Table 3. Changes of gene expression profiles in thymocytes at 24 hours and 48 hours after deletion of Bcl11b in microarray analysis.
   Table 4. PCR primers

### GENERAL DESCRIPTION

T cells develop from early T cell progenitors which have NK and myeloid potential through a series of steps, known as DN1 (double negative stage 1), DN2, DN3 and DN4, DP (double positive), and then into single positive (SP) mature CD4 or CD8 positive T cells. There are many different types of T cells including helper, cytotoxic and regulatory T cells.

Activation of T cells is brought about by interaction with appropriate antigen MHC complex. For example, helper T cells become activated when they are presented with peptide antigens by MHC class II molecules that are expressed on the surface of Antigen Presenting Cells (APCs). The process of activation of T cells is known to the skilled person.

In the present invention we show that modulation of the Bcl11b gene/gene pathway allows T cells and pro-T cells to be reprogrammed into induced T-to-Natural-Killer (ITNK) cells. Data is presented for DN, DP and SP T cells. In addition, we show that such ITNK cells are effective in the amelioration of disease in an in vivo model and do not shown any adverse effects on the animal model. The Bcl11b protein in mice and humans is highly conserved, also, T cell development in both humans and mice is very similar. This information indicates that findings in mice may be extrapolated to the treatment or prevention of human diseases.

Reference to Bcl11b herein includes any Bcl11b homologues that may be identified in other species, suitably homologues that when deleted in whole or in part can result in the generation of ITNK cells in that species.

The invention provides a method of producing induced T-to-Natural-Killer [ITNK] cells from T cells and/or pro-T cells, the method comprising modulating the activity and/or effect of at least one Bcl11b gene and/or gene product present in a T cell or pro-T cell, thereby converting said T cell and/or pro-T cell to an ITNK cell.

The invention provides a method of producing target T cells and/or target pro-T cells, the method comprising modulating the activity and/or effect of at least one Bcl11b gene and/or protein product present in a T cell and/or pro-T cell, and converting said T cell and/or pro-T cell to said target T cells and/or target pro-T cells.

Reference to T cells includes, for example, DN, DP or SP T cells such as DN1, DN2, DN3, DN4, DP thymocytes, CD4 or CD8 single positive mature T cells or γδ-T cells. Reference to pro-T cells includes common lymphoid precursor cells, stem cells and other non-T hematopoietic cells or non-hematopoietic cells which can be converted to T cells

Target T cells or target proT cells are cells which have the potential to convert into ITNK cells as a result of the modulation of the activity and/or effect of at least one Bcl11b gene and/or gene product in the T cell or pro T cell, but which have not yet converted to give the ITNK like phenotype.

Modulation of the activity or the effect of the Bcl11b gene or protein is suitably achieved by inhibiting the activity or effect of Bcl11b, either directly or indirectly.

Suitably the inhibition comprises deletion of at least part of said Bcl11b gene, suitably at least a single exon of the Bcl11b gene, suitably at least exon 4 of the Bcl11b gene. In one aspect all of the gene is deleted. Suitably, inhibition of the activity or effect of Bcl11b may be achieved by disrupting the function of Bcl11b through insertion a genetic cassette to the Bcl11b locus. Suitably, inhibition of the activity or effect of Bcl11b may be achieved by modulating epigenetic changes at the Bcl11b locus or those gene loci that regulate Bcl11b or are regulated by Bcl11b. Suitably, inhibition of the activity or effect of Bcl11b may be achieved by using antibodies (conventional or peptide Abs) to neutralize gene products of Bcl11b or its upstream or down-stream genes.

In another aspect the invention relates to genomes comprising a Bcl11b conditional knockout (cko) allele, preferably T cells or pro T cells having such a conditional mutation. The generation of conditional alleles allows the growth of cells under conditions in which Bcl11b is expressed, followed by growth under different conditions that cause the Bcl11b gene to be deleted and the ITNK phenotype to be expressed. Thus the invention also relates to a process for the induction of ITNK cells comprising activation of a conditional mutation, suitable to modulation of the activity and/or effect of at least one Bcl11b gene and/or gene product in the T cell or pro T cell.

In one aspect the modulation is directly at the level of Bcl11b gene expression, where the expression of Bcl11b is preferably inhibited to stimulate ITNK cell production. In one aspect the sequences of the Bcl11b gene, or control sequences such as promoter or enhancer regions, may be mutated, such that transcription or translation are adversely affected.

In one aspect control of the expression of Bcl11b is achieved by control of mRNA expression or protein translation. In one aspect the expression of Bcl11b is modulated by antisense RNA or the use of small interfering RNA (sRNA) or miRNA.

In one aspect modulation of Bcl11b is at the protein level. The activity of the Bcl11b protein may be modulated, preferably inhibited, by Bcl11b binding proteins, for example.

In one aspect modulating or inhibiting of the activity and/or effect of said Bcl11b gene or protein produces a downstream modulation in a biological pathway (s) in which said Bcl11b protein is involved. In one aspect said downstream modulation regulates the presence and/or activity and/or effect of a downstream target in said biological pathway. Assessment of downstream elements regulated by Bcl11b allows alternative targets to be identified which may control ITNK production from T cells and pro-T cells. The present invention also relates to identification of downstream targets—see below.

The invention provides an ITNK cell obtainable, or obtained, from a T cell or pro-T cell, including from stem cells or progenitors, wherein the T cell or pro-T cell includes a Bcl11b gene and/or gene product the activity and/or effect of which has been modulated so that the T cell or pro-T cell is capable of conversion to a ITNK cell.

The invention also provides a target T cell or target pro-T cell including at least one Bcl11b gene and/or gene product the activity and/or effect of which has been modulated when compared to the wild type cell, so that the T cell or pro-T cell is capable of conversion to an ITNK cell. The target T cell or target pro-T cell may be an ES cell, or adult stem cell, or induced pluripotent stem cell (IPS cell).

In one aspect of the invention the ITNK cells or target T/pro T cells are obtained from T cells or pro-T cells in which all or part of the Bcl11B gene has been deleted. In one aspect there is a deletion in both alleles of the Bcl11b gene, or part thereof.

The invention also relates to a mammalian genome from which all or part of the Bcl11b gene has been deleted.

The invention also relates to mature activated T cells in which Bcl11b expression is downregulated or absent (also referred to as TBcl11b-cells). Mature T cells in this context refer to normal mature T cells and not to cancerous or transformed T cells. As shown in the example section below, it has been observed by the present inventors that at a single cell level about 10-20% of activated splenic T cells have very low level of Bcl11b expression (also FIG. 6 (F)). Hence, use of these cells in medicine, particularly in the treatment of cancers and viral infections forms an aspect of this invention.

The invention also relates to cells, such as T cells and pro T cells and stem cells and animals such as non-human animals, such as a mouse, the genome of which comprises a Bcl11b conditional knockout (cko) allele.

In one aspect all or part of Bcl11b gene is floxed or otherwise associated with recombinase target sequences, to allow the Bcl11b gene or part thereof to be deleted. In one aspect the cell comprising the floxed gene expresses Tamoxifen (OHT)-inducible Cre recombinase. Expression of the Cre recombinase by OHT induction suitably causes all or part of Bcl11b to be deleted.

The invention also relates to a cell or non-human mammal in which the Bcl11b gene or protein activity has been modulated, other than by deletion, to produce an ITNK cell or target ITNK cell.

ITNK cells suitably are obtained or obtainable from another cell type (such as T cells or pro-T cells, suitably DN1, DN2, DN3, DN4, DP thymocytes, CD4 or CD8 single positive mature T cells, common lymphoid precursor cells or stem cells) and suitably exhibit one or more or all of the following properties:

(a) a morphology comparable to natural killer cells, in comparison to T cells, for example as shown in FIG. 2D, FIG. 2E and FIG. 2e.

As shown below, reprogrammed thymocytes not only expressed NK cell surface receptors but morphologically do not look like T cells, rather, they were much similar to regular NK cells which are large size, large cytoplasm, have granules and high protein synthesis activity in the abundant endoplasmic reticulum (ER) (FIGS. 2D, 2E and 2e).

(b) TCR 6 specific genomic DNA re-arrangement, for example as shown in FIG. 2C;

As shown below, certain ITNK cells have a rearranged TCR 6 locus, indicative of their origin as T cells.

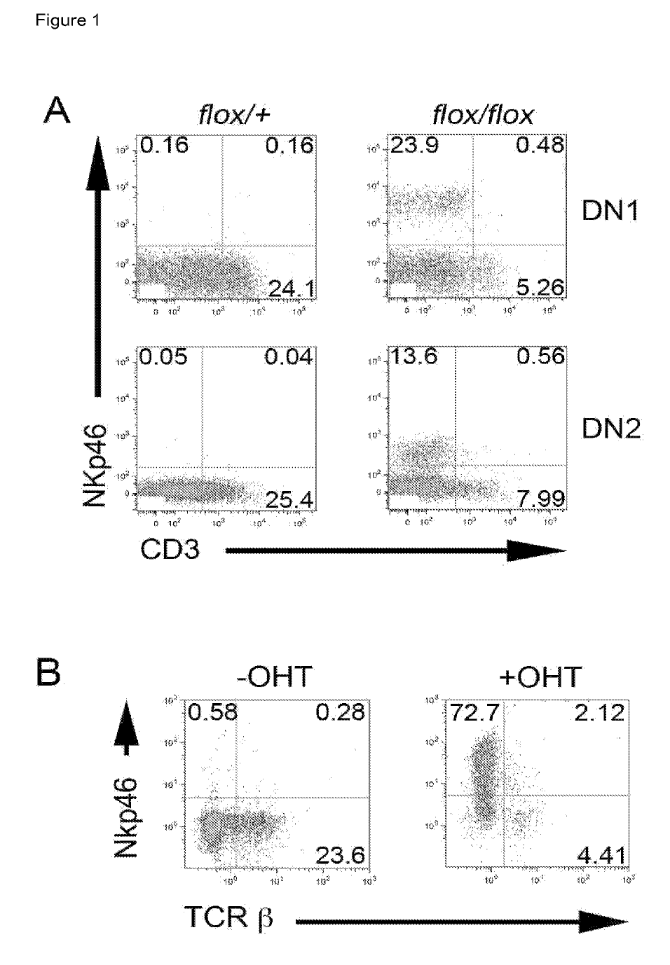

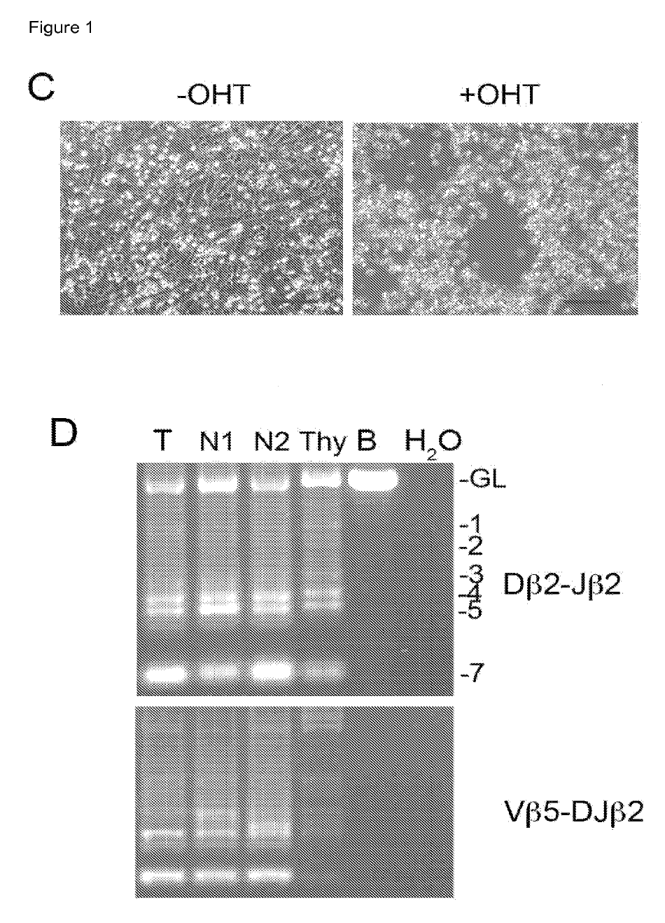

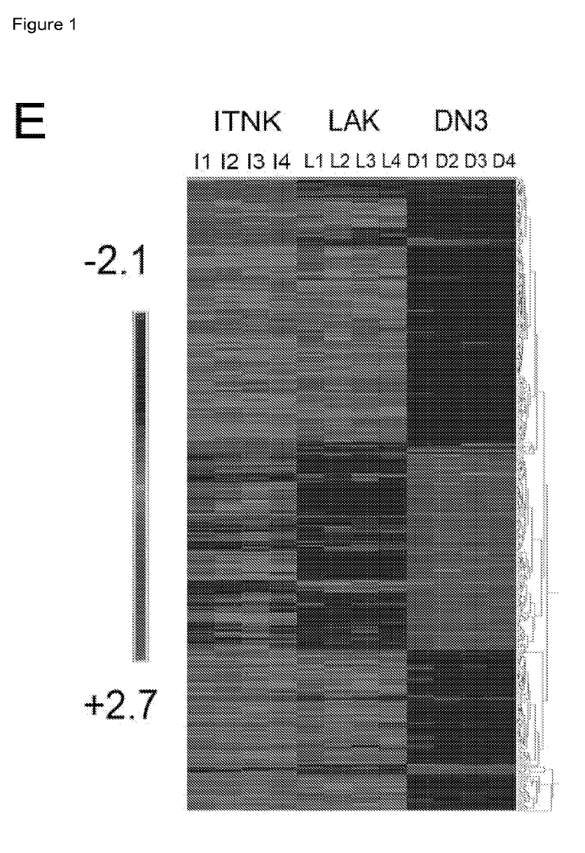

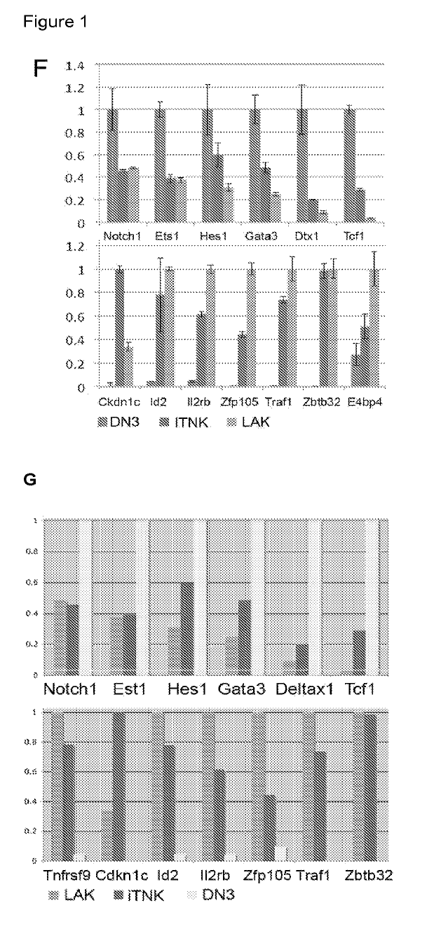

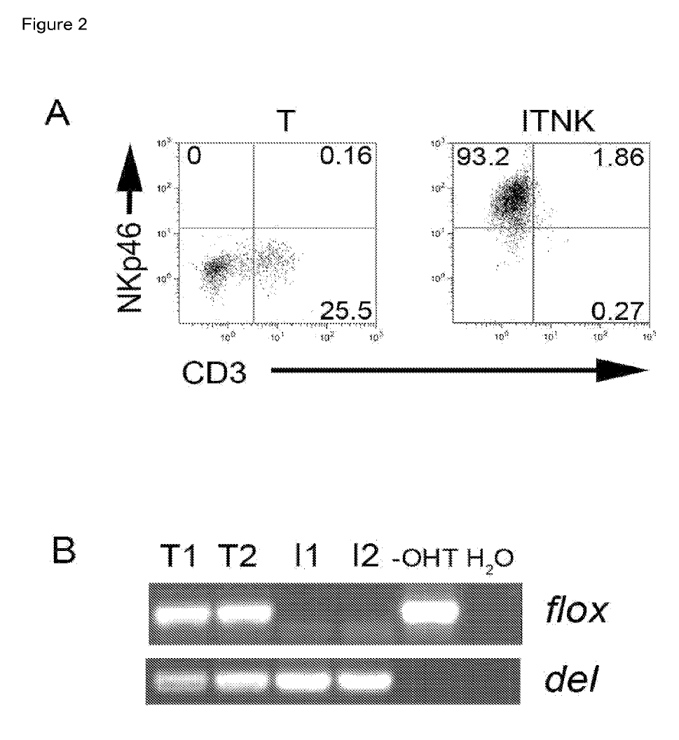

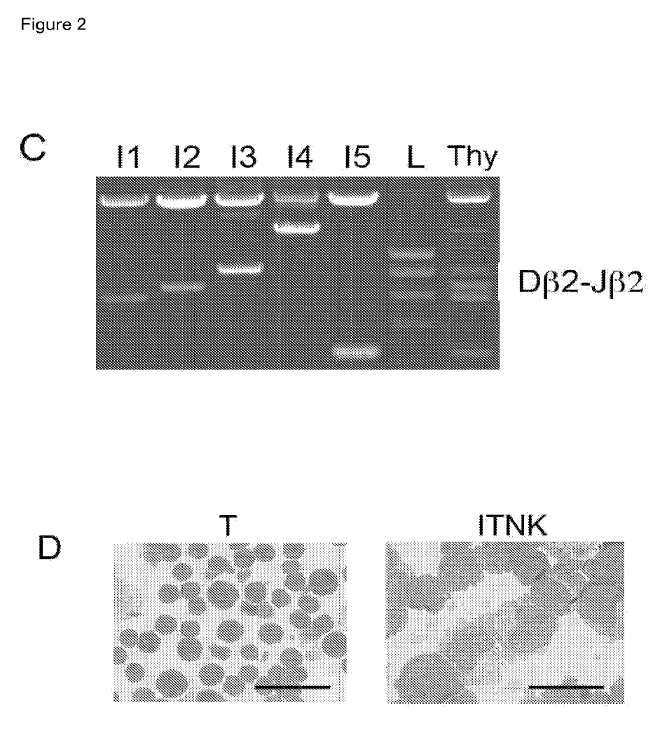

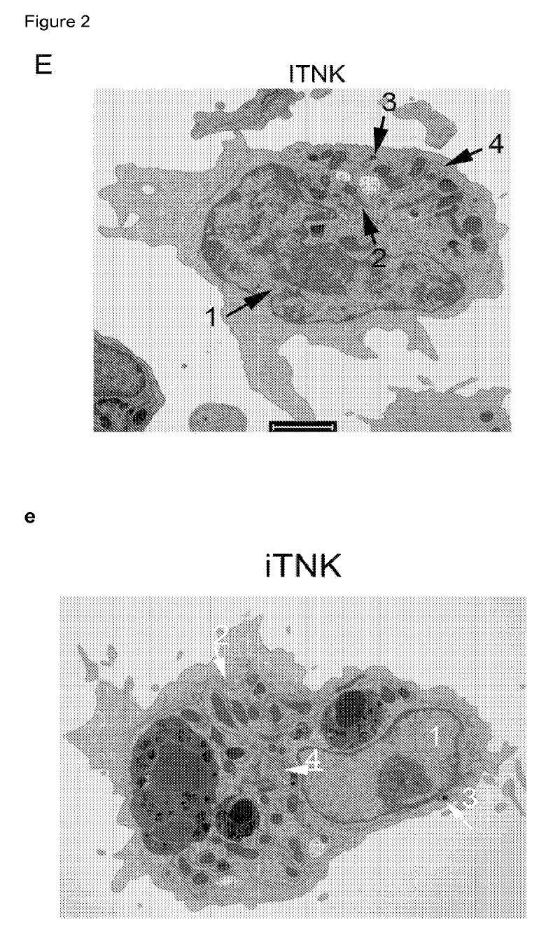

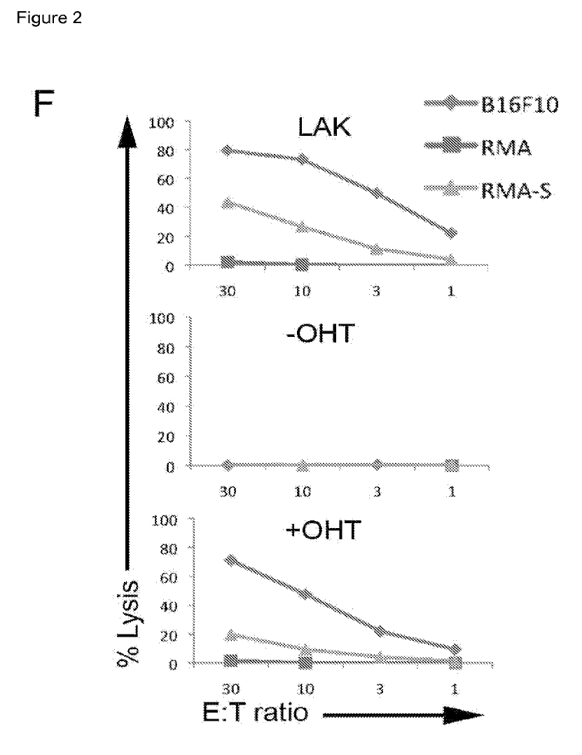

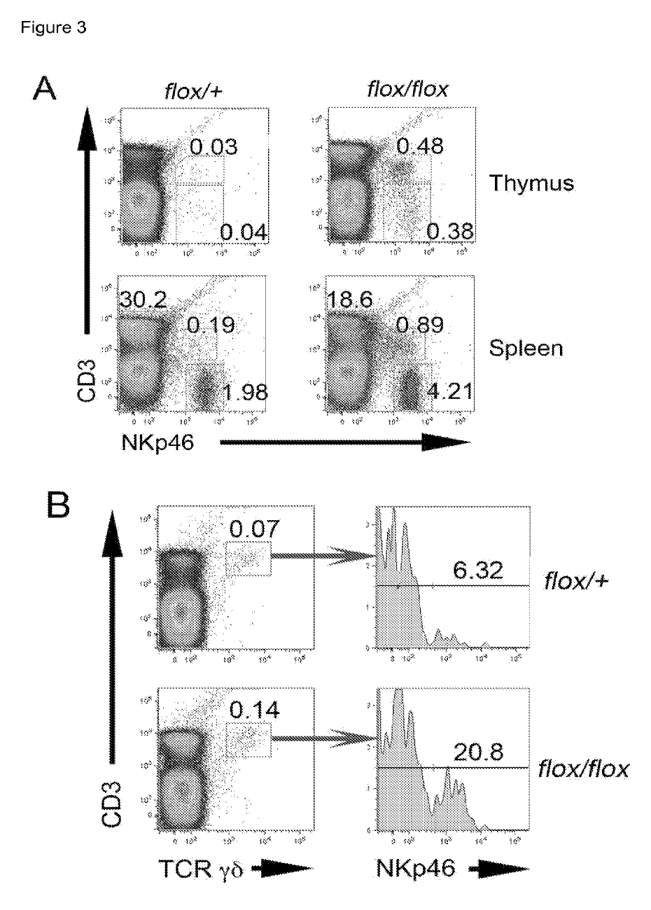

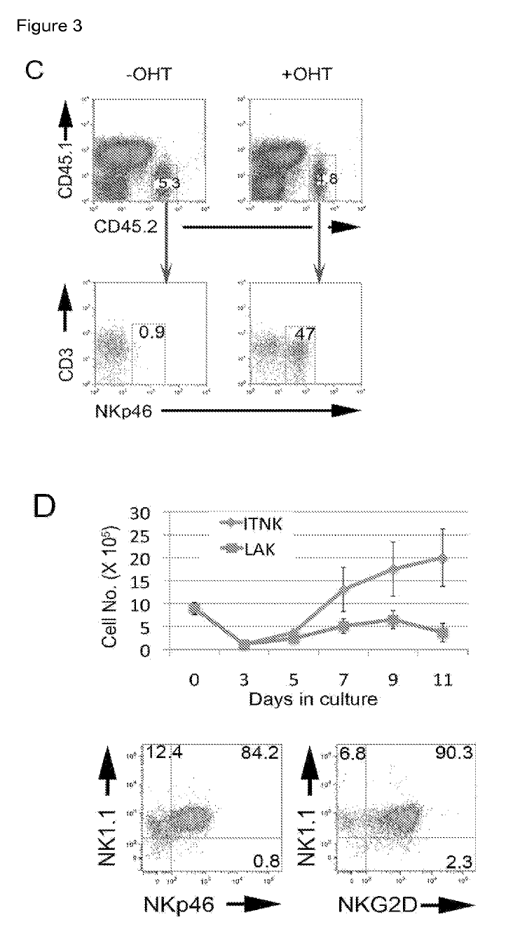

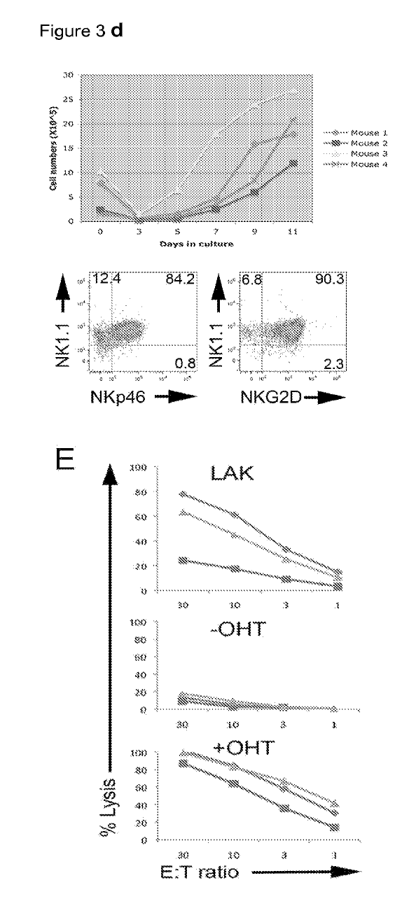

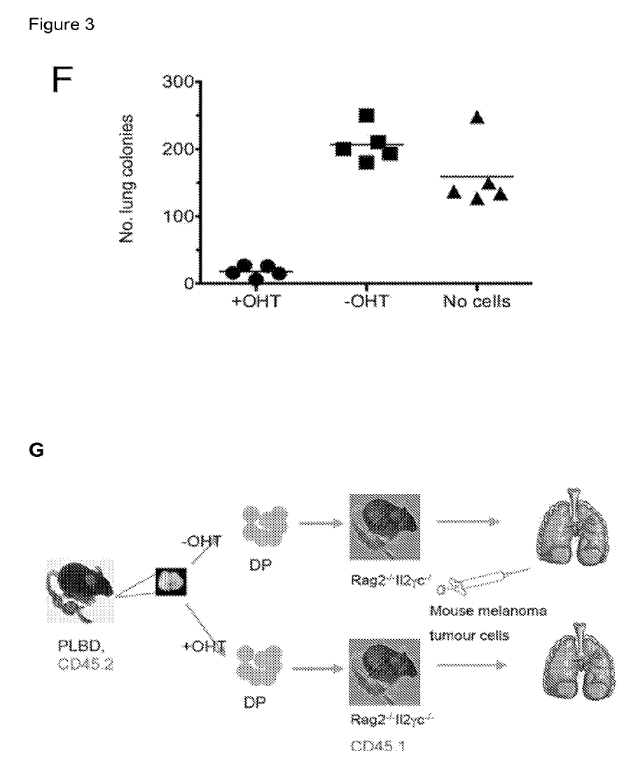

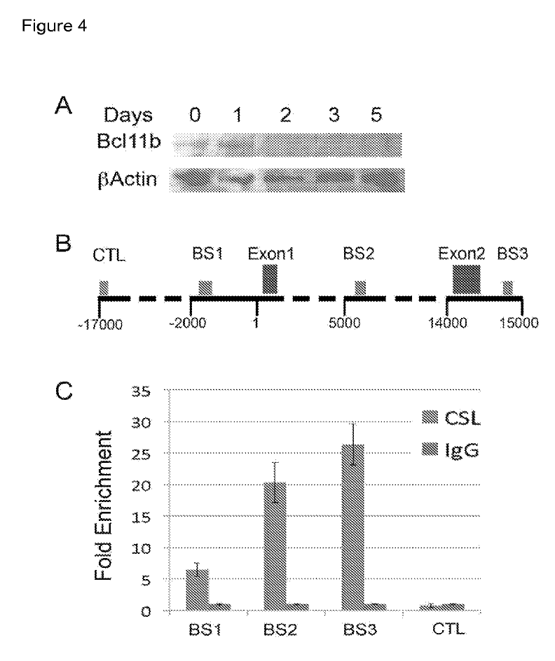

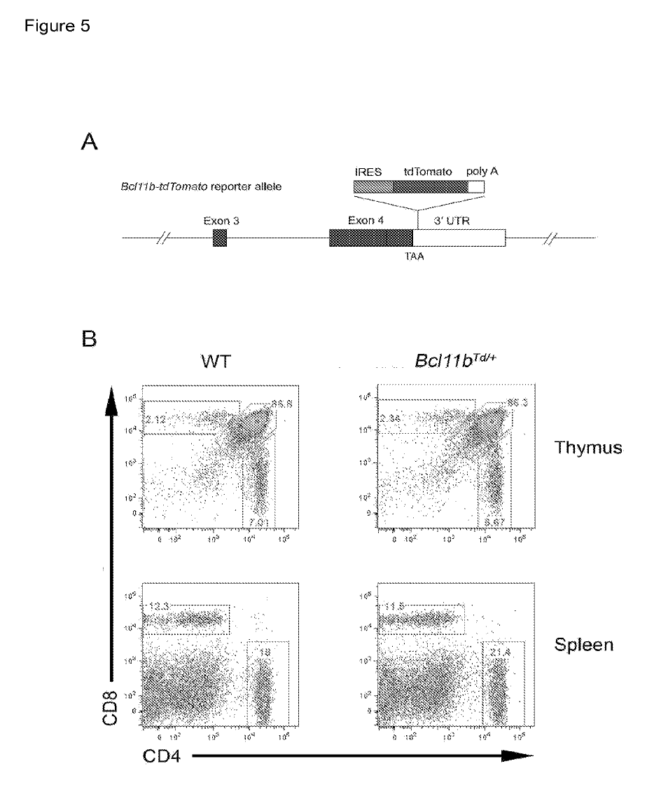

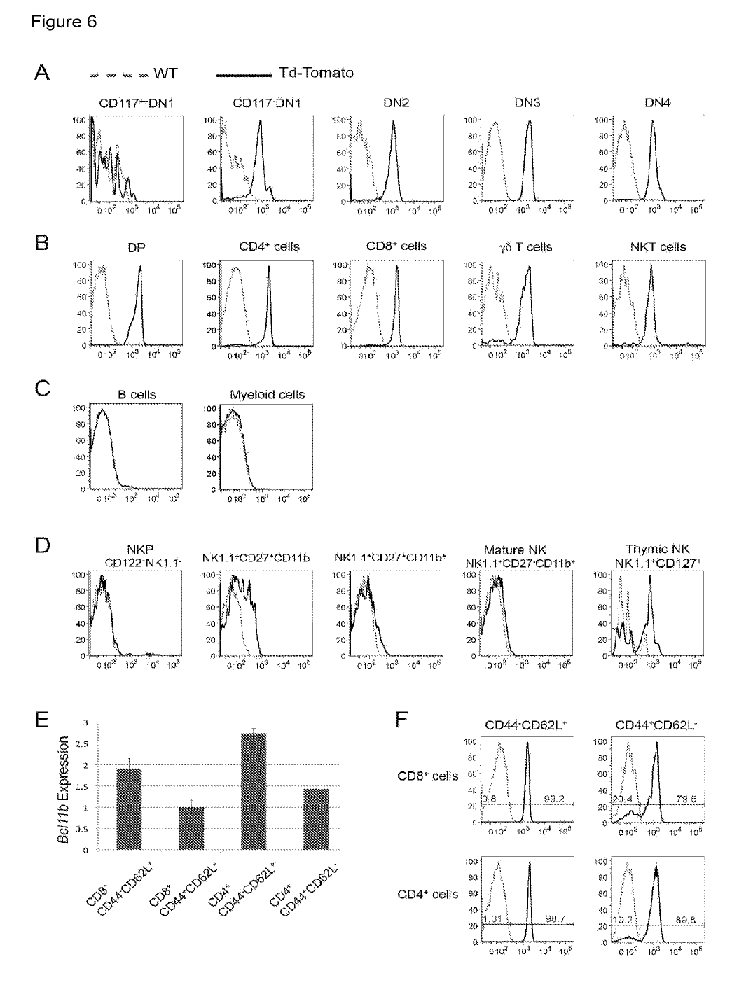

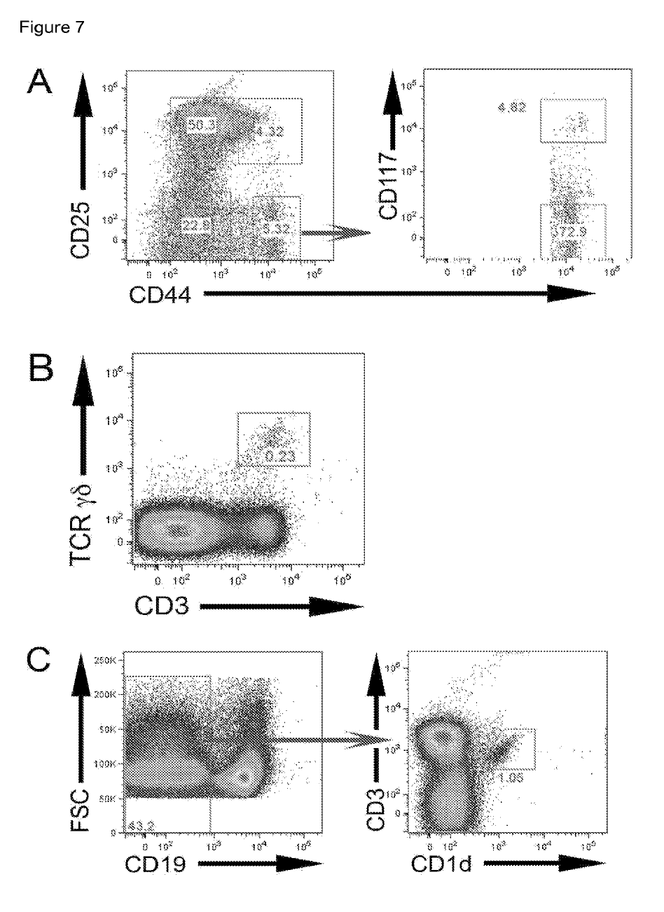

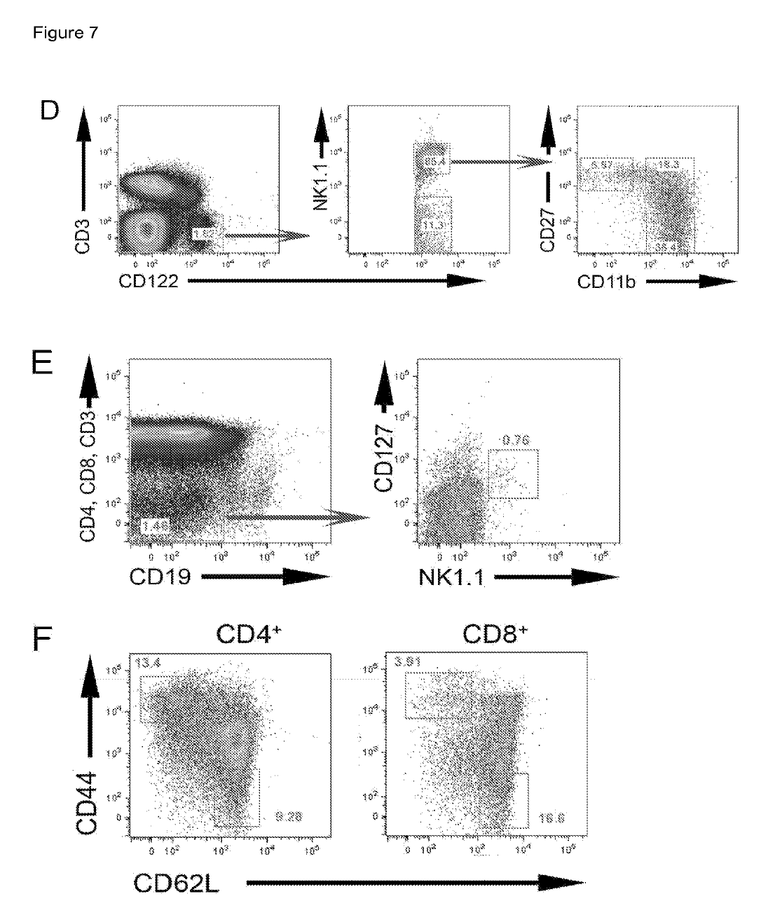

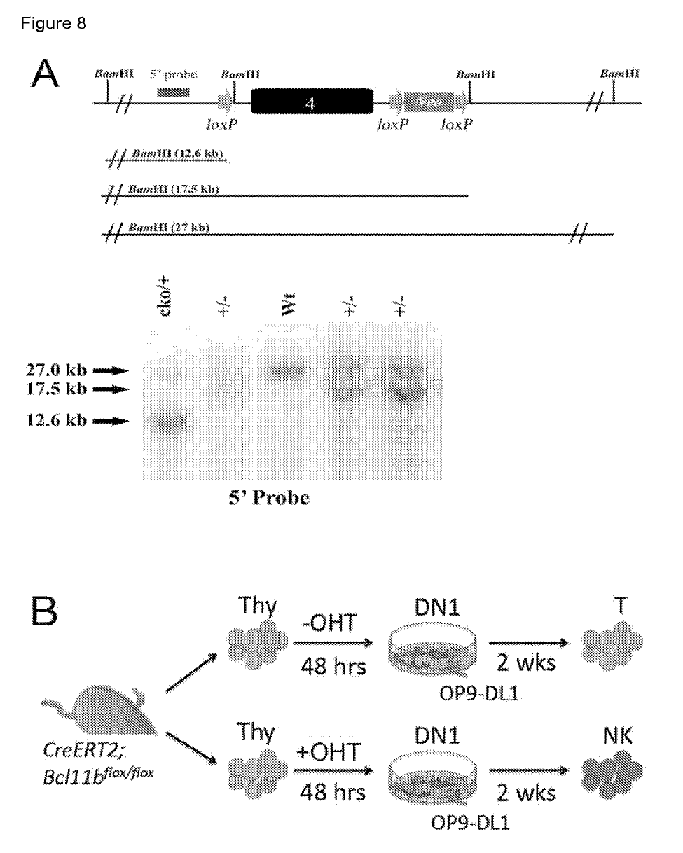

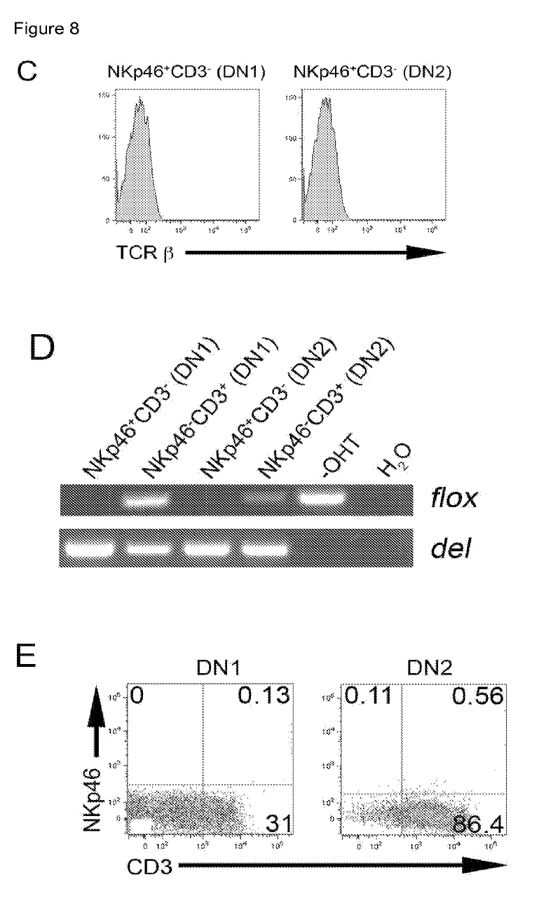

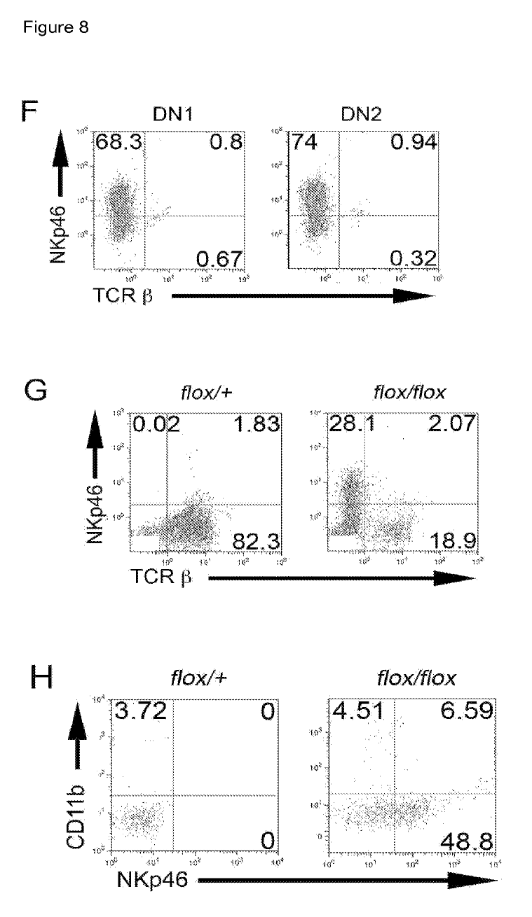

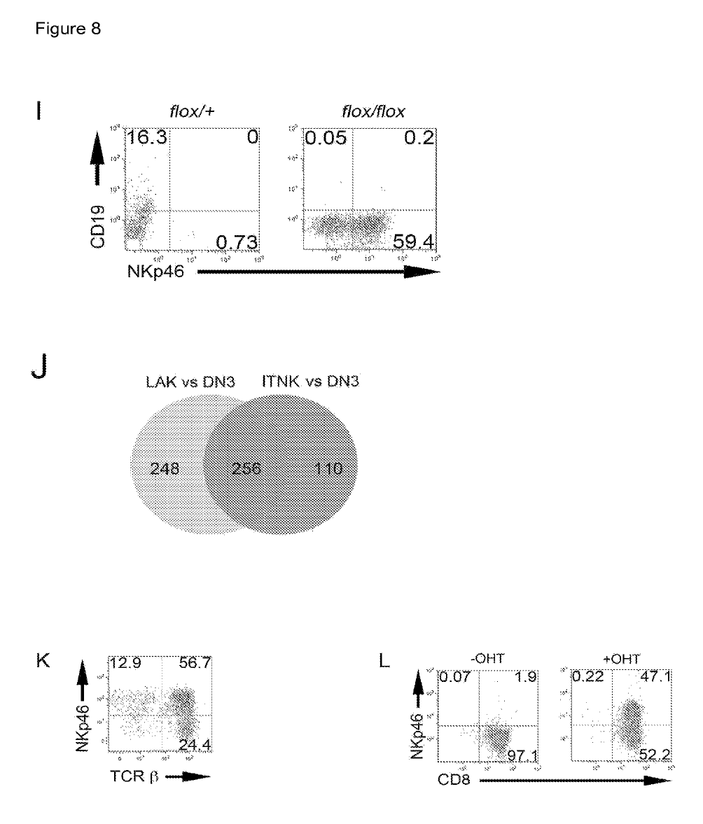

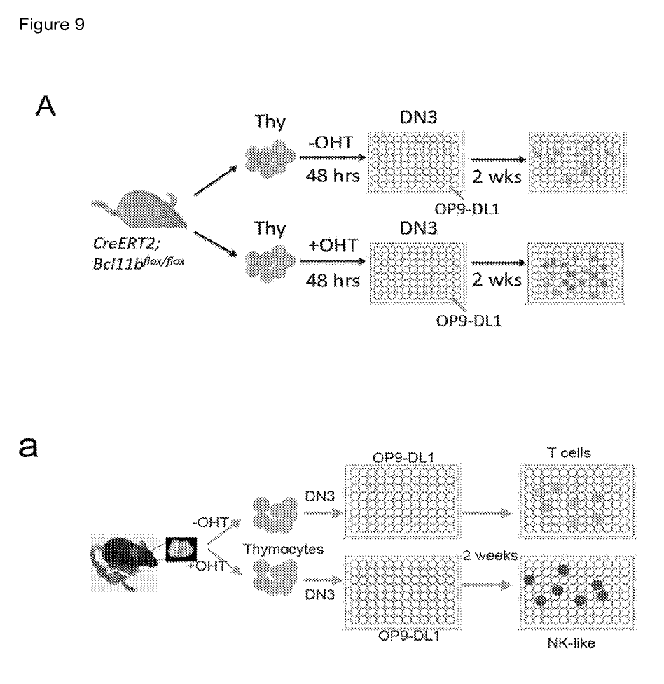

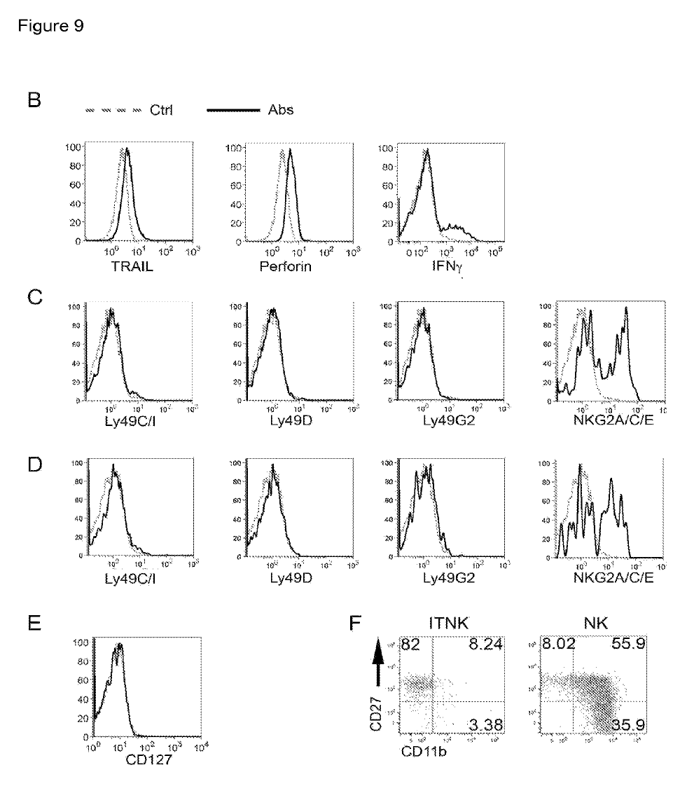

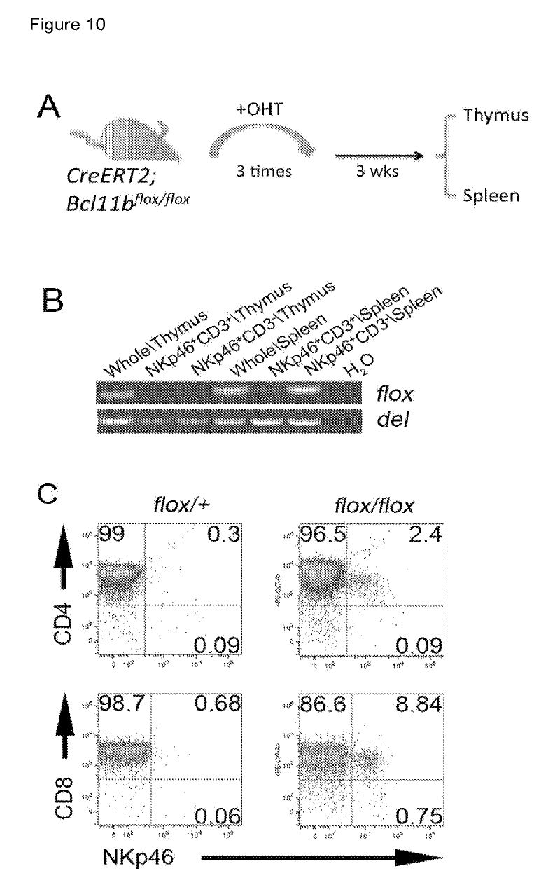

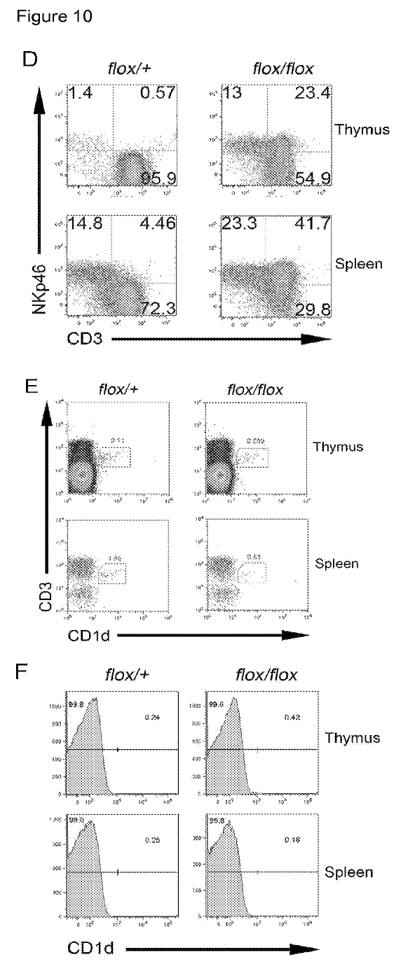

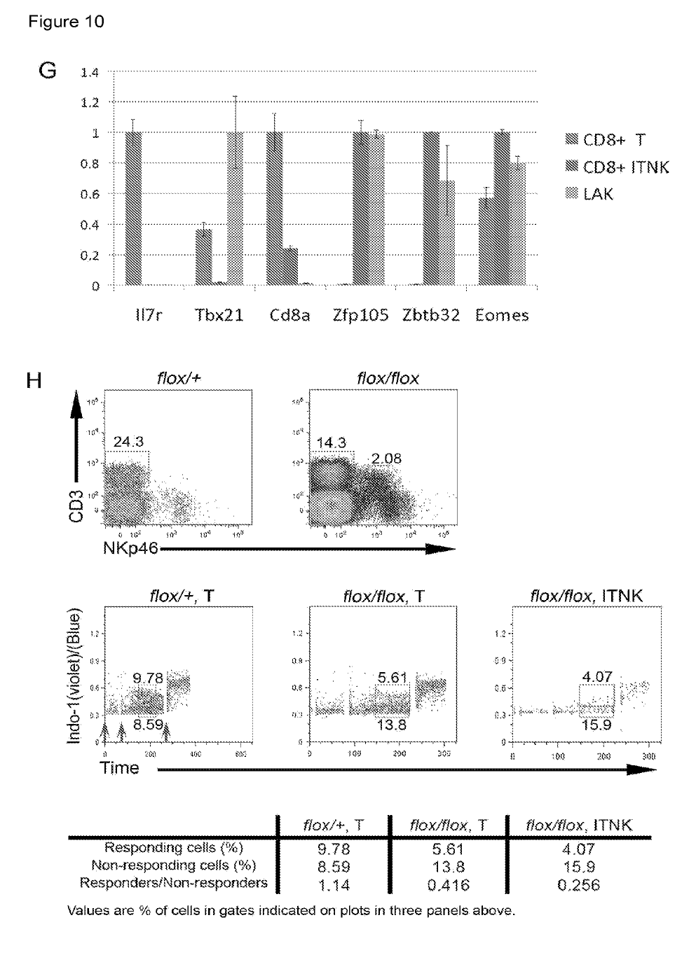

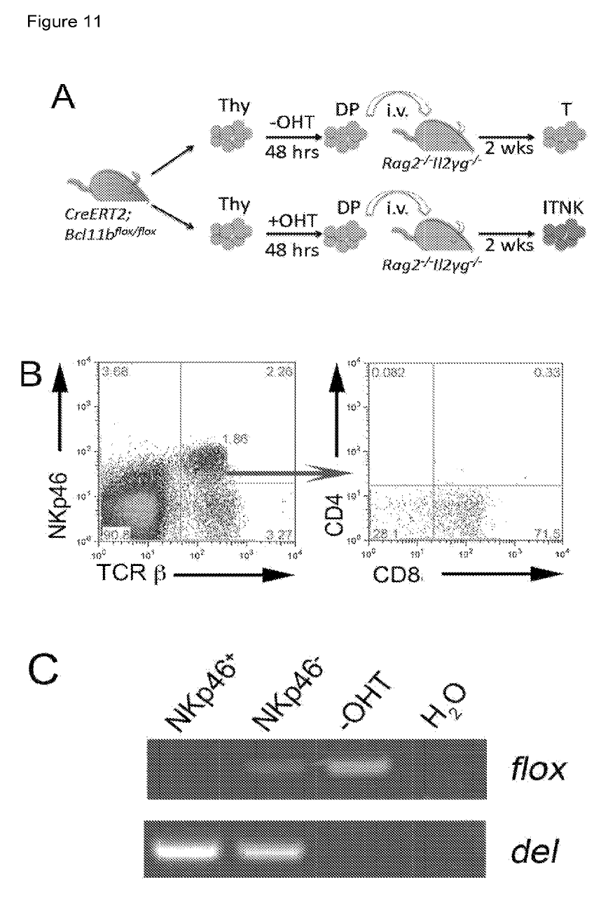

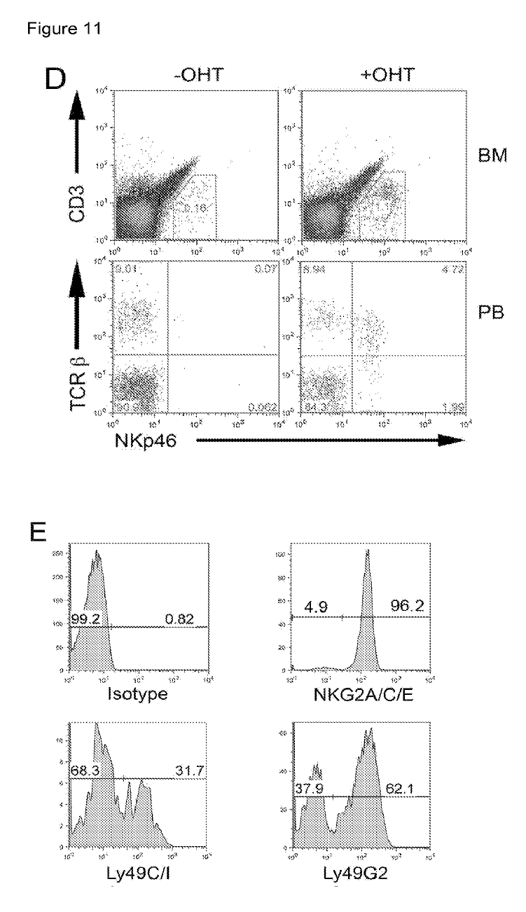

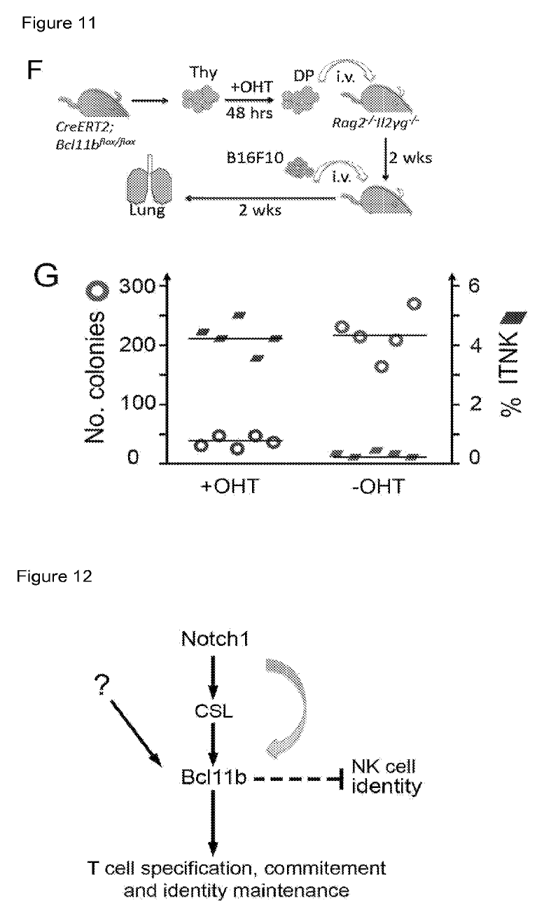

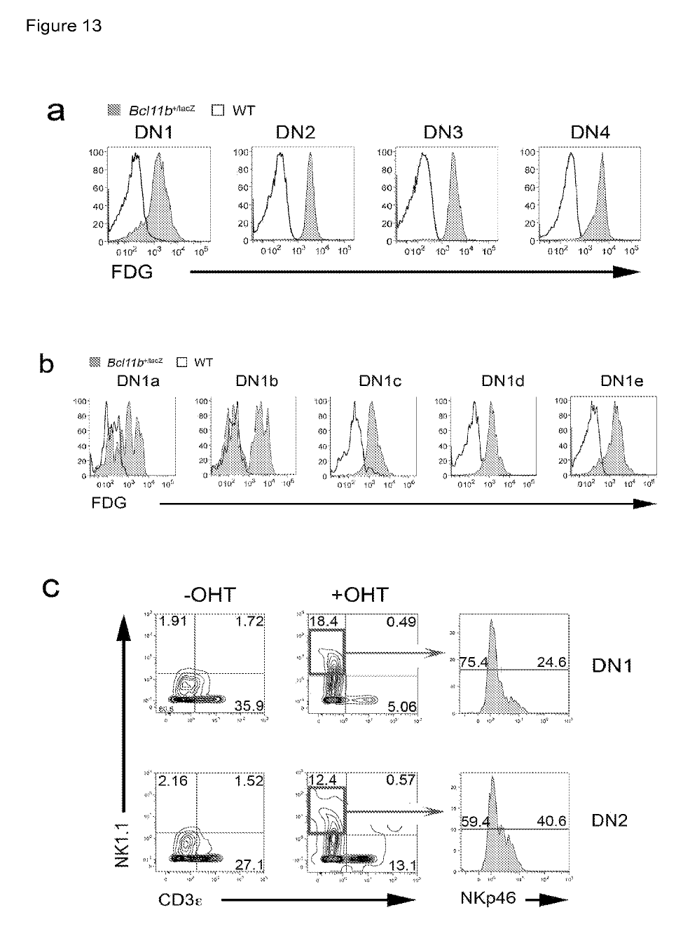

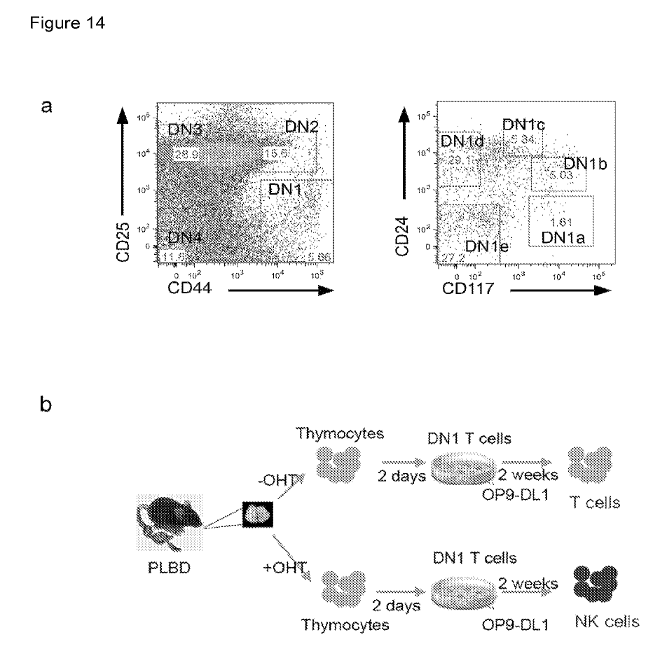

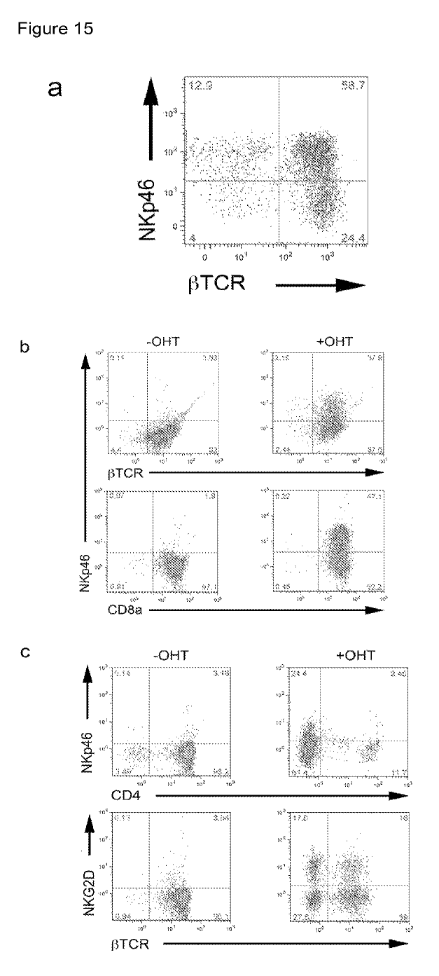

(c) a gene expression profile more similar to that of NK cells, such as LAK cells, than the parental cells from which they were developed, for example as shown in FIGS. 1E and 1G. Genes that showed an expression difference between the parental DN3 thymocytes and their Bcl11b-deficient derivatives are listed in Table 1. When considering this table of genes, ITNK cells suitably have at least 50%, suitably at least 60%, suitably at least 70% of genes differentially expressed (2 fold difference or more) in the same direction (increase or decrease) as LAK cells.

(d) cellular expression of one or more NK specific genes not found, or not expressed at high levels on non-effector or naïve T cells such as:

ZFP105, IL2Rβ, Id2, JAK1, NKG2D, NKG2A/C/E, B220, Rog (Zbtb32), Tnfrsf9, Cdkn1c, Trail, Perforin, Interferon-γ, NK1.1, NKp46, E4 bp4, NKG7, KLRD1, LTA, PLCG2, Ly49C/I and Ly49G2

(e) decreased or no expression of one or more T lineage genes, in comparison to the parent cells from which the ITNK cell was derived, such as decreased or no expression of Notch1, Est1, Hes1, Gata3, Deltaxi, TCRβ, CD3, Tcf1, IL7Ra, T-bet, CD8. In one aspect, ITNK cells are derived from CD8+ cells and do not express IL7R and/or T-bet and express low levels of CD8a.

(f) cell killing ability, for example the ability to prevent or ameliorate tumour formation or growth, the ability to kill stromal cells, tumour cells, or infected cells, suitably in comparison to the precursor cell used (parent T cells or proT cells). Cell killing may be assessed in vitro or in vivo by methods described in the Examples section herein. Additionally, the ITNKs can recognize MHC—I molecules. Moreover, the ITNK cells produced in vivo are not MHC—I restricted and are capable of killing MHC—I positive or negative cells. The ITNK cells whether produced in vitro or in vivo kill MHC—I low or negative cells.

(g) a mutation in the Bcl11b gene, or control sequences, affecting transcription, or translation or protein sequence, or otherwise affecting Bcl11b activity or effect, suitably promoting ITNK production.

Suitably the cells are capable of killing OP9-DL1 stromal cells, suitably within 2-20 days, such as 5-15 days such as 10 days after treatment to initiate the conversion from T cells or pro-T cells to ITNK cells, such as by OHT treatment. Suitably ITNKs retain a killing ability even when cultured in vitro for one month.

For the avoidance of doubt, ITNK cells produced by modulating Bcl11b activity and/or effect in a T cell and/or pro-T cell, remain ITNK cells according to the invention, if they retain cell killing ability even if Bcl11b returns to normal levels in such cells subsequently.

Suitably, ITNK cells of the invention exhibit the properties in (a), (c), (d), (e) and (f) above. Suitably, ITNK cells of the invention exhibit the properties in (a) or (c) or (d) or (e) and (f) above. ITNK cells may also possess one, or more, or all, of the following properties.

Suitably the proliferation and/or differentiation of the ITNK cells is promoted by a Supplement of IL-2 or IL-15 in the culture media.

Suitably ITNKs are able to grow out from T cell cultures within 2-20 days, such as 5-15 days, such as 10 days after Bcl11b is deleted or otherwise affected, or the Bcl11b pathway modulated suitably as assessed by the abundance of NKp46⁺ cells (FIG. 8K, 8L, 15a and 15b).

Suitably T cell/pro T cell to ITNK cell conversion from T cells/pro-T cells is greater than 50% efficient, such as greater than 60%, greater than 70%, greater than 80%, greater than 90%, greater than 95% efficient, suitably 100% efficient, by which it is meant that more than e.g. 50% of all cells in which the Bcl11b gene has been deleted, or in which the Bcl11b pathway has been otherwise modulated, go on to produce ITNK cells.

Suitably ITNK cells produced in vivo are detectable in the recipient host, such as a recipient mouse, for at least 1 month, preferably 2 months, preferably 3 months. Suitably recipient animals do not show any noticeable abnormality, indicating that the ITNK cells do not attack normal host cells in the recipient mice.

Suitably ITNK cells according to the invention possess functions of NK cells relating to regulation of the immune response, such as cytokine release.

Suitably ITNKs are able to continue proliferating for at least 3 weeks in cell culture.

In one aspect ITNK cells do not express NKp46.

Suitably ITNK cells or T cells can be independent of Notch signalling.

In one aspect the ITNK cells are not completely identical to NK cells. In one aspect ITNK cells do not express Ly49D. In one aspect ITNK cells do not express one or more T cell surface markers such as CD8, CD3e, and βTCR.

In another aspect ITNK cells express at least 20% of NK cell specific markers listed in table 2 as specific to LAK, preferably 40%, 60% or 80% of these known NK cell markers.

In one aspect, the ITNK cells produced in vivo are not MHC—I restricted and are capable of killing MHC—I positive or negative cells. The ITNK cells whether produced in vitro or in vivo kill MHC—I low or negative cells. This is explained in further detail in the example section below and shown in FIG. 3E where it is shown that unlike LAK, the in vivo produced ITNK cells killed RMA cells with almost the same efficiency as killing RMA-S. Such in vivo produced ITNKs have the advantage that their use has no risk of autoimmune diseases.

In one aspect the ITNK cells have at least 2, 3, 4 or more of the properties listed above, and preferably all such properties.

In one aspect ITNK cells demonstrate a rearranged TCR β locus, do not express all of the genes listed in the table 2 as specific to LAK, and exhibit cell killing as described herein.

In one aspect the invention provides an ITNK cell obtainable or obtained by the present invention having by a cell killing ability as assessed by methods such as those of examples 1.1.9 and 1.1.11 herein, but which do not express Ly49D.

In one aspect the NK cells comprise a suicide gene or other mechanism to allow ITNK cells to be eliminated. By way of example the genome of the ITNK cell, or T cell or pro-T cell may be engineered to contain a negative selection cassette.

The invention provides a pharmaceutical composition comprising ITNK cells together with a pharmaceutically acceptable excipient. Suitable excipients are well known in the art and include pharmaceutically acceptable buffers, preservatives, diluents and carriers and the like.

Also provided are mixtures of the ITNK cells of the invention with therapeutic agents such as anti-cancer agents or anti-infective agents e.g antiviral agents. The ITNK cells may be used in a combined preparation for simultaneous, separate or sequential use in disease therapy such as anticancer or antiviral therapy, although the use of ITNKs is not limited to cancer and antiviral therapy, and ITNKs might be useful for eliminating many types of abnormal cells. For example, ITNKs may also be used for treatment or prophylaxis of bacterial, yeast and parasite infections.

Suitable anticancer agents include alkylating agents, antimetabolites, anthracyclines, plant alkaloids, topoisomerase inhibitors, and other drugs affect cell division or DNA synthesis and function in some way. Other drugs include targeted therapies such as monoclonal antibodies and tyrosine kinase inhibitors and nanoparticles. Furthermore, also suitable are drugs that modulate tumor cell behaviour without directly attacking those cells, such as hormone treatments, known as an adjuvant therapy. As an alternative, agents for immunotherapy may also be included, such as use of interferons and other cytokines to induce an immune, and vaccines to generate specific immune responses.

Suitable anti-infectives include drugs that act to block viral entry into cells, drugs that prevent virus replication, such as reverse transcriptase inhibitors, integrase inhibitors, Protease inhibitors, and drugs that prevent virus release into the body.

Delivery of cells and compositions of the invention may be by any suitable route of administration including enteral or parenteral, such as by injection or infusion, for example in a once a day, once a week, once a month, or other suitable schedule. Multiple or single rounds of treatment may be employed.

The invention relates to a method for the preparation of a medicament for a human or non-human mammal comprising taking a sample of T cells, and converting the T cells to ITNK cells as described herein, optionally then using said cells in a medicament for treatment. Optionally the method comprises dilution or otherwise selection of a single T cell, and optionally manipulation of the T cell genome prior to use as a medicament.

The invention provides ITNK cells and target T/pro-T cells for use in medicine, and use of ITNK cells and target T/pro-T cells in the preparation of a medicament for the treatment or prophylaxis of disease, such as cancer or viral infection. ITNKs may also be used for treatment or prophylaxis of bacterial, yeast and parasite infections.

The invention also provides mature activated T cells in which Bcl11b expression is down-regulated or absent (also referred to as TBcl11b-cells) for use in medicine, and use of such cells in the preparation of a medicament for the treatment or prophylaxis of disease, such as cancer or viral infection.

NK cells play a major role in the rejection of tumors and cells infected by viruses and the ITNK cells of the present invention demonstrate anti cancer properties in vitro and in vivo. In one aspect ITNK cells produced from T cells or pro T cells are used to treat diseases such as cancer and infectious diseases such as viral infections.

The ability to convert T cells or pro-T cells into ITNK cells and use of TBcl11b-cells allows therapies to be developed using a patient's own cells, which can be used in the same patient without rejection.

The invention thus relates to use of a therapeutically effective amount of ITNK cells derived from the T cells or pro-T cells of a patient in the treatment or prevention of infection or disease in that individual. In a further aspect the cells may be used in another individual.

The invention provides a method of treating a patient, the method comprising administering to said patient a therapeutically effective amount of ITNK cells or TBcl11b-cells preferably wherein the ITNK cells are derived from T cells or pro-T cells that have been obtained from the patient.

Target T cells or pro-T cells may also be employed as above, in place of ITNK cells.

In one aspect, T cells/pro-T cells or target T cells or target pro-T cells of the invention do not refer to cancerous or transformed T cells.

In one aspect the ITNK cells according to the invention are obtained by modulating Bcl11b activity and/or effect in transformed or cancerous T cells, such as T cells from lymphoma patients, which may have different levels of Bcl11b as compared to wild type cells. In this aspect, the transformed or cancerous T cells are the T cells/pro-T cells or target T cells or target pro-T cells capable of conversion to ITNK cells.

In one aspect ITNK cells do not show any adverse effects on the patient.

In one aspect, the invention provides a method of isolating naturally occurring mature activated T cells in which Bcl11b expression is downregulated or absent (TBcl11b-cells) from a patient, expanding the cells in vitro and administering to the patient a therapeutically effective amount of the TBcl11b-cells for treatment of conditions such as cancer and viral infections.

In one aspect, the invention provides a method of isolating T cells/pro-T cells from a patient (human or non-human); modulating the activity and/or effect of the Bcl11b gene and/or gene product so that the T cell or pro-T cell is capable of conversion to ITNK cells; administering to the patient a therapeutically effective amount of ITNK cells or target T cells or target pro T cells for treatment of conditions such as cancer and viral infections.

In one aspect the ITNK cells are derived from a single T cell which is converted into ITNK cells using the methods described herein. This process suitably allows for a T cell specific for an antigen of interest, such as a disease specific antigen, such as a viral or microbial antigen or such as a tumour-specific antigen, to be converted into an NK-like cells.

From a single T cell up to 0.5 million ITNKs can be obtained. This is a much higher number as compared to human NK cells where approximately 1600 cells can be produced by proliferation of a single NK cell.

The invention relates to modulation of Bcl11b directly, and also use of components of the Bcl11b pathway and modulators thereof in the production of ITNK cells.

An appreciation that T cells and pro-T cells can be converted to ITNK cells allows this conversion to be used as an assay for compounds that might be used to control the conversion process. Thus the invention relates to an assay for identification of a compound which assists in the reprogramming of T cells to ITNK cells, the method comprising contacting T cells or pro-T cells with a test compound and then monitoring or selecting for the conversion of T cells to ITNK cells. Such compounds could include small chemical molecules, proteins (including but not limited to growth factors, cytokines, antibodies) or nucleic acid based therapies, and libraries of any of these compounds. The invention also relates to use of compounds so identified in the conversion of T cells or pro-T cells to ITNK cells and additionally to those compounds per se.

In addition the invention relates to an assay for identification of a genetic mutation which controls the reprogramming of T cells to ITNK cells, the method comprising random or targeted mutation of T cells or pro-T cells and screening for ITNK cells or selection of ITNK cells under conditions where T cells or pro-T cells are not viable.

An appreciation that Bcl11b plays a role in the conversion of T cells and proT cells to ITNK cells allows the Bcl11b gene and protein to be used directly as probes to identify other components in the Bcl11b signaling pathway, which may then be tested for an effect on conversion of T cells to ITNK cells. Thus the invention relates to an assay for identification of a compound which assists in the reprogramming of T cells to ITNK cells, the method comprising screening for compounds that bind to the Bcl11b gene or the Bcl11B protein, and further optionally assessing whether said compounds are able to promote the conversion of T cells to ITNK cells. The invention further relates to use of compounds so identified in the conversion of T cells or pro-T cells to ITNK cells and those compounds per se.

In a yet further aspect the invention relates to the use of factors which regulate the Bcl11b gene or protein expression or activity, or which are functionally downstream of the Bcl11b gene or protein, or which are functionally upstream of the Bcl11b gene, to effect the conversion of T cells to ITNK cells, and to the use of modulators of these factors to effect the conversion of T cells to ITNK cells. Suitably, the modulators are antibodies targeting Bcl11b or factors which regulate the Bcl11b gene or protein expression or activity or downstream gene products or upstream gene products. Suitably, the modulators are administered to human or non-human diseased subjects.

For example, Notch is upstream of Bcl11b. In one aspect modulators of Notch signalling are used to effect a conversion of T cells and proT cells to ITNK cells.

CSI acts upstream of Bcl11b. In one aspect modulators of CSL are used to effect a conversion of T cells and proT cells to ITNK cells.

In another aspect the invention relates to an assay for identifying a downstream target for Bcl11b, the assay comprising monitoring the effect of modulating the Bcl11b gene and/or protein product on a putative downstream target. Such an assay may further comprise monitoring conversion of T cells or pro-T cells to ITNK cells when the downstream target per se has been modified. Such an assay may further comprise identifying a modulator which either interacts with said downstream target so as to modulate the activity and/or effect thereof, to result in the conversion of a T cell or pro-T cell to one or more ITNK cells.

The invention further provides for a non-human animal carrying ITNK cells, and/or target T cells or target pro-T cells.

In one aspect ITNK are independent of Notch signalling.

In a further aspect the invention relates to a method of stimulating T cell production, the method comprising modulating the activity and/or effect of at least one Bcl11b gene and/or protein present in a pro-T cell, such as a human or embryonic stem cell, or IPS cell. Suitably the method comprises stimulating the Bcl11b expression or activity.

An understanding of the importance of Bcl11b in the T cell maturation pathway suggests that manipulation of the Bcl11b gene or protein expression or activity can help to stimulate T cell production. The present invention thus relates to use of activators of the Bcl11b pathway, either upstream or downstream, in the stimulation of T cells production, either in vivo or in vitro, and use of T cells so produced in medicine.

### EXAMPLES

T cells develop in the thymus and are critical for adaptive immunity. Natural killer (NK) lymphocytes constitute an essential component of the innate immune system in tumor surveillance and defense against microbes and viruses.

General Introduction to T and NK cell development

T cell development involves progenitor homing, lineage specification and commitment, and requires a complex interplay among key transcription factors (1, 2). The earliest populations of thymocytes, which lack T cell receptor (TCR) co-receptors CD4 and CD8 (double negative or DN cells) (28), can be further subdivided by cell surface markers as DN1-4 (29). The DN1 (CD44⁺CD25⁻) thymocyte population contains multipotent progenitors (30, 31) whereas DN2 thymocytes (CD44⁺CD25⁺) have NK and myeloid potential (30, 31). These non-T cell developmental potentials are lost in the DN3 (CD44CD25⁺) thymocytes. DN4 thymocytes (CD44⁻CD25⁻) have undergone have undergone β-selection after successful Tcrβ gene rearrangement (32) and already initiated the process of differentiating to the CD4⁺CD8⁺ double positive (DP) stage (33, 34).

In the periphery, the cytokine IL-7 and the constant interaction of T cells with self peptide-MHC play a critical role in T cell maintenance (3). RT-PCR analysis indicates that many genes important for T cell commitment start to increase their expression in the transition from DN1 to DN2, with Bcl11b being the most upregulated transcription factor (4). In bony fish, Bcl11b is shown to be required for T cell precursor homing to the thymus (5). In the mouse, Bcl11b has critical roles in fetal thymocyte development and survival, and in positive selection and survival of double-positive thymocytes (6, 7).

NK cell committed precursors (CD122⁺) differentiate from multipotent haematopoietic progenitors primarily in the bone marrow but differentiation can also occur in the thymus and secondary lymphoid tissues (35). These precursors give rise to NKp46⁺ immature NK cells, which subsequently express additional receptors as they differentiate, including MHC receptors, NKG2A/C/E and Ly49s (36, 12). Besides their participation in innate immune responses, NK cells have recently been shown to possess some adaptive immune features (37).

Although NK developmental pathways are not entirely clear, two subsets of NK cells, bone marrow-derived (CD127⁻) and thymic (CD127⁺) NK cells have been identified in the mouse that differ in development sites and origins (Huntington et al., 2007). Previous studies have identified molecules important for NK cell development and homeostasis. For example, Id2, which antagonizes the bHLH E proteins E2A and HEB, is essential for the NK lineage since the Id2-knockout mice lack NK cells (Ikawa et al., 2001; Yokota et al., 1999). Conversely, forced expression of Id2 or Id3 is able to re-direct pro-T cells to NK cell differentiation (Blom et al., 1999; Fujimoto et al., 2007). A recent study also identifies Zfp105 as a NK specific transcription factor since overexpressing it promotes differentiation from hematopoietic stem cells to the NK lineage (Chambers et al., 2007).

Several genes or pathways important for T cell development genes also have functions for NK cells. For example, Gata3 and T-bet plays important roles in NK development, maturation and homeostasis (Samson et al., 2003; Vosshenrich et al., 2006) (Townsend et al., 2004). Notch triggers initiation of T cell program, and is required to sustain or protect the cells throughout the pro-T cell stages (Maillard et al., 2005; Radtke et al., 1999; Rothenberg, 2007). Loss of Notch signalling in DN1 thymocytes convert them into dendritic cells (Feyerabend et al., 2009). Deleting of Notch in the thymus leads to accumulation of B cells in the thymus possibly by a cell-extrinsic pathway (Feyerabend et al., 2009; Radtke et al., 1999).

In contrast to its role in T cells, Notch generally suppresses NK potential in DN1 and DN2 pro-T cells until the cells progress to the committed DN3 stage (Carotta et al., 2006; De Smedt et al., 2005; Garcia-Peydro et al., 2006; Rolink et al., 2006; Schmitt et al., 2004; Taghon et al., 2007; van den Brandt et al., 2004). Nevertheless, it is proposed that transient Notch signaling is required for NK differentiation from early progenitors or stem cells (Benne et al., 2009; Haraguchi et al., 2009; Rolink et al., 2006). This may reflect the role of Notch in promoting T/NK bipotent progenitors (DeHart et al., 2005).

In the periphery, the cytokine IL-7 and the constant interaction of T cells with self peptide-MHC play a critical role in T cell maintenance (3). RT-PCR analysis indicates that many genes important for T cell commitment start to increase their expression in the transition from DN1 to DN2, with Bcl11b being the most upregulated transcription factor (4). In bony fish, Bcl11b is shown to be required for T cell precursor homing to the thymus (5). In the mouse, Bcl11b has critical roles in fetal thymocyte development and survival, and in positive selection and survival of double-positive thymocytes (6, 7).

Bcl11b is a C₂H₂ zinc finger transcription repressor (Avram et al., 2000; Cismasiu et al., 2005). Germline mutation of Bcl11b in the mouse causes thymocyte developmental block at the DN3 stage secondary to apoptosis induced by defective β-selection in thymocytes (Wakabayashi et al., 2003). Bcl11b is recently shown to be required for positive selection and survival of double-positive thymocytes (Albu et al., 2007). However, suppression of Bcl11b expression by RNA interference selectively induces apoptosis in transformed T cells but does not appear to affect normal mature T cells (Grabarczyk et al., 2007).

Here we show that the transcription factor Bcl11b was expressed in all T cell compartments, and was indispensable for T lineage development. When Bcl11b was deleted, T cells from all developmental stages acquired NK cell properties and concomitantly lost or decreased T cell-associated gene expression. These Induced T-to-Natural-Killer (ITNK) cells, which were morphologically and genetically similar to conventional NK cells, killed tumor cells in vitro and effectively prevented tumor metastasis in vivo. Therefore ITNKs may represent a new cell source for cell-based therapies.

### Bcl11b is Expressed and Required in the Early T Cell Progenitors

Microarray studies indicate that expression of many genes important in T cell commitment, including Bcl11b, starts to increase in DN2 thymocytes. Among transcription factors, Bcl11b is the most drastically upregulated in the transition from DN1 to DN2 (Rothenberg, 2007). To determine Bcl11b expression in early T cells at the single cell level, we produced a lacZ knock-in allele of Bcl11b where a SA-lacZ cassette is inserted into the intron 3 to trace its expression (Song-Choon Lee, et al, unpublished). Therefore, Bcl11b expression can be traced indirectly by using Fluorescein di-3-D-galactopyranoside (FDG), a fluorescent substrate of 3-galactosidase, in flow cytometry. In hematopoietic lineages, expression of Bcl11b was only detectable in T cells (data not shown). In the thymus, almost all DN2-DN4 thymocytes expressed Bcl11b (FIG. 13a and FIG. 14a). In contrast, only about 80% of DN1 thymocytes expressed Bcl11b. Further analysis using a CD117 antibody identified that 60% of DN1a and DN1b thymocytes, which are thought to be the earliest T cell progenitors (Porritt et al., 2004), already expressed Bcl11b (FIGS. 13B and 14a), suggesting a possible role of Bcl11b at the earliest T lineage specification steps.

To determine Bcl11b expression in T cells at the single cell level, we produced and analyzed a Bcl11b tdTomato knock-in mouse (FIG. 5A-B). In hematopoietic lineages, Bcl11b was not expressed in B or myeloid cells whereas almost all DN2-DN4 and DP thymocytes, CD4⁺ and CD8⁺ T cells, γδ-T cells and Natural Killer T cells (NKT) expressed Bcl11b (FIG. 6, A-C and 7, A-C). In DN1 thymocytes, very little to no expression of Bcl11b was detected in CD117⁺⁺ cells (known as Early T-cell-lineage Progenitors (2)) (FIGS. 6A and 7A). During NK development, transient, low Bcl11b expression was observed in immature NK cells but not in NK precusors (NKP) or mature NK cells (FIGS. 6D and 7D). In contrast, the majority of thymic NK cells, identified by CD127 (8), expressed Bcl11b (FIGS. 6D and 7E). Moreover, in both CD4⁺ and CD8⁺ splenic T cells, Bcl11b transcript was reduced roughly two-fold in activated T cells (CD44⁺CD62⁻L) compared to naïve (CD44⁻CD62⁺L) cells in quantitative real time-polymerase chain reaction (qRT-PCR) analysis (FIGS. 6E and 7F) and exhibited a bimodal pattern of expression (FIG. 6F).

Bcl11b Deletion Caused Loss of T Cell Identity and Acquisition of Nk-Specific Properties in T cells

The above expression and function data have demonstrated that Bcl11b is expressed in T cell precursors and required for differentiation to T cell lineage. Germline deletion of Bcl11b caused apoptosis in DN3 thymocytes in the fetal thymus but did not obviously affect DN1/2 cells (Wakabayashi et al., 2003). To further determine Bcl11b functions in T cells, we generated the conditional knockout mice (Bcl11b^{flox/flox}) where exon 4 was floxed (FIG. 8A), which were crossed to the Rosa26Cre-ERT2 mice (9). All the thymocytes from CreERT2; Bcl11b^{flox/flox} mice express Tamoxifen (OHT)-inducible Cre recombinase. Consequently, in CreERT2; Bcl11b^{flox/flox} mice (PLBD line. Referred to as flox/flox in the manuscript), Bcl11b could be deleted by treating cultured cells or mice with Tamoxifen (OHT). From OHT-treated whole thymocytes from these and the control (CreERT2; Bcl11b^{flox/+}, referred to flox/+) mice, we sorted and subsequently cultured DN1 and DN2 cells in T cell media (Flt3 ligand and 11-7) for 2 weeks (FIG. 14b) on OP9-DL1 stromal cells (FIG. 8B) (10), which support T cell development but suppress NK cell development from the progenitors (11). OP9-DL1 stromal cells express Delta-Like-1 Notch ligand and support robust T cell development (Schmitt and Zuniga-Pflucker, 2002) while normally suppressing NK cell development (Rolink et al., 2006; van den Brandt et al., 2004). All stromal cells were killed in the OHT-treated flox/flox DN1 thymocyte culture.

Flow cytometry showed that 18% of the cultured thymocytes now expressed the NK cell marker NK1.1 (DN1 in FIG. 13c). 24% of cells in this culture expressed NKp46, which is primarily expressed on NK cells (FIG. 1A) (12). These NKp46⁺ cells did not express T cell genes CD3 or TCRβ (FIG. 8C), and had lost both alleles of the Bcl11b exon 4 (FIG. 8D), indicating that they did not acquire or had lost T cell features despite being co-cultured with OP9-DL1 stromal cells for 14 days. PCR genotyping of these NK1.1⁺CD3⁻ and NKp46⁺CD3⁻ cells showed that they had deleted both alleles of the Bcl11b exon 4 while those NKp46⁻CD3⁺ cells from the same OHT treated culture were found to still retain at least one copy of the Bcl11b cko allele. On the other hand, the control OHT-treated flox/+ and untreated flox/flox DN1 cells proliferated rapidly, and many (36%) acquired CD3 expression but not NK1.1⁺ or NKp46⁺ (FIG. 1A and FIG. 8E) consistent with Notch signalling suppressing NK development and excluding the possibility that the NKp46⁺ cells in OHT treated DN1 cell culture were derived from NK precursor contamination (FIG. 13c). These data thus demonstrated that Bcl11b deficiency caused production of the NKp46⁺ cells from DN1 thymocytes and that Bcl11b was required in early T cell development.

T cell lineage commitment is thought to occur in DN2 cells with increased expression of T cell specification genes such as Gata3, Tcf1 and Bcl11b (Ciofani and Zuniga-Pflucker, 2007; Rothenberg, 2007). Nevertheless, recent data suggest that even DN2 thymocytes still retain differentiation potentials of myeloid and NK lineages (Bell and Bhandoola, 2008). We next investigated Bcl11b function during T cell lineage commitment by deleting Bcl11b in purified DN2 thymocytes. Wild type DN2 thymocytes (−OHT) proliferated extensively on OP9-DL1 cells and gave rise to CD3⁺ cells but no NK cells (−OHT DN2 in FIG. 13c). Similar to cultured DN1 thymocytes, OHT-treated flox/flox DN2 thymocytes also produced NKp46⁺CD3⁻ cells which killed the stromal cells, whereas control DN2 thymocytes did not (FIG. 1A and FIG. 8E). Similar to that in DN1 thymocyte culture, NK1.1⁺CD3⁻ and NKp46⁺CD3⁻ cells also grew out from Bcl11b-deficient DN2 thymocytes culture on OP9-DL1 stromal cells (+OHT DN2 in FIG. 13c), demonstrating rapid loss of T cell differentiation potential upon Bcl11b loss in the DN2 thymocytes.

Growth of NK-like cells from Bcl11b-deficient DN1 or DN2 thymocytes appeared to be Notch signaling independent since NKp46⁺ cells were readily produced from DN1 or DN2 thymocytes cultured on OP9 stromal cells without IL-2 (FIG. 8F). Hence, Bcl11b has an essential function in the initial specification of the T cell lineage.

We subsequently deleted Bcl11b in DN3 thymocytes. Again, stromal cell-killing NKp46⁺CD3⁻ cells appeared (FIG. 1B-C; FIG. 8G). We purified DN3 thymoytes from OHT treated whole thymocytes from CreERT2; Bcl11b^{flox/flox} and cultured them on OP9-DL1 stromal cells. Within 14 days of culturing, most of the cells became NKp46⁺CD3⁻ and were able to kill stromal cells. Supplement of IL-2 or IL-15 in the culture media greatly promoted proliferation and/or differentiation of these cells. Consequently most cells in the culture were NKp46′ and they started to kill stromal cells within 10 days after OHT treatment (FIGS. 1B and 1C). NK progenitors normally do not differentiate on OP9-DL1 stromal cells. (FIG. 1D).

The reprogramming also worked in myeloid or B cell culture media (FIG. 8H-I), demonstrating that reprogramming to NKp46⁺ cells was intrinsic to the Bcl11b-deficient thymocytes. To further confirm that the NKp46⁺CD3⁻ cells came from T cells, we purified them and examined their TCRβ locus for DNA rearrangements. These NKp46⁺CD3⁻ cells retained TCR β V(D)J recombination even though they no longer expressed Tcr β on the cell surface, thus genetically confirming the T cell origin of these NKp46⁺CD3⁻ cells (FIG. 1D). We thus named these killer cells that were reprogrammed from T cells as Induced T-to-Natural-Killer or ITNK cells.

We next compared using microarray analysis the expression profiles of DN3 thymocytes, normal splenic NK cells that were expanded in vitro after enrichment (lymphokine-activated killer, or LAK cells, composed of >90% NK cells), and ITNKs reprogrammed from DN3 cells (FIG. 1E). Consistent with the killing ability of ITNK cells, their expression profile was much more similar to that of LAK cells than to their parental DN3 thymocytes. Genes that showed expression difference between the parental DN3 thymocytes and their Bcl11b-deficient derivatives were listed in Table 2. qRT-PCR analysis was subsequently performed to confirm the array results (FIG. 13F). qRT-PCR validation showed that expression of many T lineage genes, such as Notch1, Est1, Hest Gata3, Dtx1 and Tcf1 was decreased, whereas expression of genes usually associated with NK cells such as Id2 (13), IL2rβ (CD122), Zfp105 (14) and E4 bp4 (15) was upregulated (FIG. 1F and table 1). Zbtb32 (Rog, Repressor of GATA), which is not normally expressed in DN3 cells, but plays important roles in regulating T cell activation and suppresses Gata3 activity (16), was highly expressed in ITNKs. Expression of Cdkn1c (p57KIP2), a putative direct downstream target gene of Bcl11b (17), was also drastically increased in ITNKs (FIGS. 1F and 1G). Indeed, p57KIP2 expression was not barely detectable in DN3 cells but drastically increased in DN3 derived iTNKs (FIGS. 1F and 1G). Further analysis from the array data identified 504 genes that were expressed at least two folds higher in LAKs vs DN3 thymocytes, and 366 genes in DN3 thymocyte-derived NKp46⁺CD3⁻ cells vs their parental DN3 thymocytes (Table 2). 70% of these 366 genes in iTNKs were found overexpressed in LAKs (FIG. 8J). These results thus collectively demonstrated that Bcl11b was essential for maintaining the T cell expression profile and for suppressing NK cell gene expression.

We next investigated whether Bcl11b was required for T cell identity maintenance in all T cells by subjecting purified double positive (DP) thymocytes, CD4 or CD8 single positive mature T cells, to OHT treatment. These cells were then cultured on OP9-DL1 stromal cells. Similar to cultured Bcl11b-deficient DN3 thymocytes, iTNKs grew out from all T cell cultures within 10 days after Bcl11b was deleted, as demonstrated by many NKp46⁺ cells (FIG. 15a, 15b, 15c). Interestingly, these iTNKs that were derived from Tcrβ-expressing T cells, still retained Tcrδ on the cell surface. In contrast to iTNKs from CD8⁺ T cells that still expressed CD8, the CD4⁺ single-positive T cell-derived iTNKs did not express CD4 anymore (FIG. 15c).

ITNKs could also be produced from mature T cells. We OHT-treated sorted double positive (DP) thymocytes, CD4⁺ and CD8⁺ T cells, and γδ-T cells from flox/flox mice. Many ITNKs (NKp46⁺) were found growing in DP thymocytes and CD8⁺ T cell cultures (FIG. 8K-L), which effectively killed stromal cells. These ITNKs, in contrast to those reprogrammed from DN1-3 thymocytes, retained TCRβ on the cell surface. We were unable to obtain consistent production of NKp46⁺ cells from splenic or thymic CD4⁺ T cells, or from γδ T cells, because these cells appeared prone to cell death in vitro once Bcl11b was deleted.

### Once Bcl11b Deleted, all DN3 Thymocytes Lost T Cell Identity and Became ITNK

To estimate the reprogramming (T to NK conversion upon Bcl11b deletion) efficiency, we sorted single DN3 thymocytes from OHT-treated flox/flox thymocytes into individual wells of 96-well plates pre-seeded with OP9-DL1 stromal cells in T cell media (FIG. 9A). Out of the 79 wells that had cells growing, 36 wells had many fast-proliferating T cells which expressed T cell surface markers including CD3 and Tcrβ (FIG. 2A). PCR genotyping confirmed that cells in these wells did not have complete Bcl11b deletion—but deleted only one fox Bcl11b allele (FIG. 2B, lanes T1 and T2). These cells (flox/−) nevertheless served as excellent controls for Cre toxicity because they had activated Cre recombinase. In the other 43 wells, thymocytes were reprogrammed to NKp46⁺ stromal cell-killing ITNKs (FIG. 2A). In these 43 wells, cells grew relatively slow but killed stromal cells. Still, from one DN3 thymocyte, up to 0.5 million of stromal-killing cells were readily obtained 14 days post OHT treatment. Flow cytometry analysis showed that almost all the cells in these wells expressed NK-specific markers NKp46 and thus were ITNKs (FIG. 2A). IL-2 was clearly able to greatly promote proliferation of ITNKs because from one DN3 thymocyte, up to 0.5 million ITNKs were obtained with IL-2, but only about 50,000 cells without IL-2. All ITNK cells had lost both Bcl11b alleles (FIG. 2B, lanes 11 and 12), and ITNKs of individual wells possessed unique rearranged TCRβ loci thus confirming their independent origins (FIG. 2C). Therefore, once Bcl11b was deleted, the reprogramming efficiency of DN3 thymocytes to ITNKs could reach 100%. ITNKs from DN3 thymocytes not only expressed NK cell surface receptors and possessed similar cytotoxic functions, but were morphologically similar to LAK cells which are larger than T cells, have granules and high protein synthesis activity with abundant endoplasmic reticulum (FIG. 2, D-E).

ITNKs were larger than thymocytes and had granules and showed evidence of high protein synthesis activity with abundant endoplasmic reticulum (FIG. 2, D-E). Besides NK1.1 and NKp46, ITNKs expressed NKG2A/C/E, TRAIL, perforin and interferon-γ, but not some other key NK cell function genes, such as members of the Ly49 family or FasL (CD178) (FIG. 9B-C). Similar observations were made with in vitro reprogrammed ITNK cells from DP thymocytes (table 2 and FIG. 9D). ITNKs were unlikely to be related to thymic NK cells since they did not express CD127 (FIG. 9E). Moreover, unlike conventional mature NK cells, most ITNKs did not express CD11b, rather, they expressed CD27, and retained killing ability even after being cultured in vitro for one month (FIG. 9F). The iTNKs from in vitro cultured Bcl11b deficient DN3 thymocytes killed OP9-DL1 stromal cells after overnight co-culture. In fact, iTNKs retained the killing ability even cultured in vitro for at least a month. Transferring of supernatant of the iTNK cells culture to fresh stromal cells did not kill these cells, therefore cytokines secreted by iTNK cells were not sufficient, and cell-cell contact was required, for efficient killing.

We next measured the killing ability of the DN3-reprogrammed ITNKs by performing standard ⁵¹Cr-release assays with three NK-sensitive cell lines: B16F10 melanoma (MHC—I low or negative) (18), RMA lymphoma, which express MHC class I molecules, and RMA-S lymphoma (TAP-1-deficient variant), which have reduced MHC class I presentation (19, 20). LAK cells generally only killed MHC-class I negative cells (FIG. 2F). Similar to LAKs, ITNKs also selectively killed MHC—I negative B16F10 and RMA-S cells, but did not kill MHC—I positive RMA lymphoma cells (FIG. 2F). Compared to regular LAKs, iTNKs appeared to have relatively lower killing potency. This is consistent with a lack of the full NK cell surface repertoire in the in vitro derived ITNKs (Table 2). We speculated that an in vivo microenvironment might be required for fully converting Bcl11b deficient T cells to more potent tumour cell killers.

### In Vivo Reprogrammed NK Cells are More Potent Tumour Cell Killers

To exclude the possibility that ITNKs were in vitro artifacts, we deleted Bcl11b in vivo (FIG. 10A). Two to three weeks after OHT treatment, ITNKs were detected in both the spleen (NKp46⁺CD3⁺) and the thymus (NKp46⁺) from flox/flox mice but not the fox/+ controls (FIG. 3A). Bcl11b was found deleted in these in vivo reprogrammed ITNKs (FIG. 10B). Importantly, both CD4⁺ and CD8⁺ ITNKs (NKp46⁺) were found (FIG. 10C). Some wild type γδ-T cells expressed NKp46, however, Bcl11b deletion caused a 3-fold increase in the NKp46⁺γδ-T cells (FIG. 3B), which suggested that all T cell populations have reprogramming potential. The in vivo reprogrammed ITNKs could readily be expanded in NK culture conditions (FIG. 10D), but they were not NKT cells (FIG. 10E-F). Besides expressing NK cell-associated genes, the in vivo reprogrammed ITNKs also lost or decreased some key T cell genes such as Il7ra, Tbx21 (T-bet), Cd8 (FIG. 10G). Consequently, TCR signaling in ITNKs appeared to be compromised (FIG. 10H).

The in vivo analysis of ITNKs in flox/flox mice was complicated by the presence of many host T cells and NK cells (FIG. 3A). To address this problem, and also to investigate whether in vivo reprogramming upon Bcl11b loss is cell autonomous, we transplanted 2-4 million OHT-treated DP thymocytes from flox/flox mice (CD45.2⁺) into Rag2^{-/-}Il2rg^{-/-} mice (CD45.1⁺) that lack B, T and NK cells (FIG. 11A) (21). We chose DP thymocytes because they usually account for more than 75% of total thymocytes and could be efficiently reprogrammed in vitro to ITNKs (FIG. 8K). Two weeks after transplantation, around 5% of splenocytes were found to be from the donor cells (CD45.2⁺) (FIG. 3C), and approximately 47% of them expressed NKp46 and thus were ITNKs. ITNKs lost both copies of Bcl11b and the majority of them expressed CD8 (FIG. 11B-C). The other 53% cells (NKp46⁻) were T cells and still retained the Bcl11b floxed allele (FIG. 11C). The ITNKs usually accounted for 2-3% of total splenocytes. Interestingly, the majority of the splenic NKp46⁺ ITNKs expressed CD8 (FIG. 11B). Significant amount of NKp46⁺ ITNKs were also present in the bone marrow and peripheral blood (FIG. 11D). We estimated there were about 200,000 iTNK cells in the spleen alone. Nevertheless, this low ITNK number was unexpected because 2-4 millions of DP thymocytes were initially transplanted and because the T to ITNK conversion in vitro was 100%. It is possible that most of the Bcl11b-deficient DP thymocytes died either before or immediately following the conversion due to the difference between the in vivo microenvironment and in vitro culture condition, for example, the relative low levels of cytokines in the mice. No NKp46⁺ cells were found in control mice transplanted with untreated DP thymocytes (FIG. 3C). ITNK cells were maintained in the recipients for at least 3 months without change in cell number, perhaps representing a dynamic balance in their numbers. Importantly the recipient mice did not show any noticeable abnormality, indicating that ITNK cells did not indiscriminately kill normal cells nor were malignantly transformed.

The in vivo iTNKs were further phenotyped by flow cytometry. Compared to those reprogrammed in vitro, the in vivo reprogrammed ITNKs appeared to express more NK surface receptors such as NKG2A/C/E and most receptors of the Ly49 family including Ly49C/I and Ly49G2 (FIG. 11E) (table 2), and could be extensively expanded ex vivo with IL-2 or IL-15 for at least 3 weeks while still retaining their killing ability (FIG. 3D). NK surface receptors such as Ly49 family genes including Ly49C/I, Ly49G2 were absent in the in vitro derived iTNK cells. Importantly, these iTNK cells were not NKT cells because CD1d-restricted NKT cells do not express NKp46 (Walzer et al., 2007), and the iTNKs examined in this study did not express V β 2TCR which is present in many NKT cells and recognizes non-polymorphic CD1d molecule (data not shown) (Bendelac et al., 2007).

Regular NK cells become LAKs in culture with cytokines and can be expanded for up to 7 days. After that, LAKs gradually lose proliferation and killing ability. To test the proliferation capacity of the in vivo iTNK, we cultured 2 millions splenocytes (containing approximately 50,000 iTNKs) from recipient mice in LAK condition. Most cells died in the first 3 days (FIG. 3d). However, within 7 days of culturing, we obtained about 2 millions NKp46⁺Tcrβ⁺ ITNKs which accounted for 80-90% of the cell population and were able to continue proliferating for at least 3 weeks (FIG. 3d).

To assess functions of the in vivo iTNK cells, we used the ex vivo expanded iTNKs from the recipient mice to investigate their tumour-cell killing ability. Consistent with their expressing more killer effectors and receptors, the in vivo iTNK cells were much more potent in killing tumour cells than the regular LAKs, even after extensive ex vivo expansion These cells exhibited elevated cytotoxic potential and were also generally more potent than both in vitro ITNKs and LAKs against each of the target cells (FIG. 3E, and FIG. 2F). Unexpectedly, these in vivo iTNK were potent killers for all three tumour cell lines tested, regardless of their MHC—I expression status. They killed RMA cells with almost the same efficiency as killing RMA-S cells (FIG. 3E), despite expression of some inhibitory Ly49 receptors which recognize MHC—I. Transplantable murine melanoma B16 cell lines are well-established models for studying experimental cancer therapies and NK cell tumour surveillance function (22). Injection of B16 cells into Rag2^{-/-}Il2rg^{-/-} mice leads to rapid formation of metastatic foci in the lungs (23). To investigate the tumour-killing ability of the ITNK cells in vivo, we injected two million OHT-treated or -untreated DP thymocytes from flox/flox mice into Rag2^{-/-}Il2rg^{-/-} recipients to allow reprogramming of thymocytes to ITNKs in vivo (FIG. 11F). Two weeks later, each recipient was injected with 50,000 B16F10 melanoma cells. Four weeks after the initial thymocyte transplantation, recipients were sacrificed and analyzed. Mice injected with PBS or with untreated DP cells had about 200 metastatic foci in the lungs. In contrast, mice injected with OHT-treated DP thymocytes had approximately 20 tumour colonies in the lung (FIG. 3F and FIG. 11G). Therefore ITNKs were potent killers of tumour cells in vivo and prevented cancer progression.

### Bcl11b Regulated by Notch Signalling in T Cells

Western blot indicated that in the thymocytes from CreERT2; Bcl11b^{flox/flox}, the Bcl11b protein levels decreased drastically 24 hours after OHT treatment. And 48 hours later, Bcl11b protein was undetectable. Hence, deletion of BcIllbled to rapid disappearance of Bcl11b protein (FIG. 4A). To probe gene expression changes immediately following Bcl11b deletion in T cells, we performed expression array analysis 24 and 48 hours following OHT treatment. Microarray analysis showed that in OHT-treated flox/flox thymocytes, expression of T cell genes such as TCRβ and CD3 was already down-regulated within 24 hours (table 3). In another 24 hours, many genes associated with NK cells were expressed (table 3). Table 3 lists genes that Bcl11b loss significantly affected their expression (2 folds). Expression of several genes that are important for NK cell functions, such as NKG7, KLRD1 (CD94), PLCG and IFNG, were already increased 48 hours after OHT treatment.

Bcl11b is proposed to be regulated by Notch signaling in T cell development (24). Recent genome-wide ChIP-seq in Drosophila has indeed identified CG6530, the Drosophila orthologue of Bc111 gene, is a direct downstream target gene of Notch signalling (Krejci et al., 2009). Notch signalling normally plays an inhibitory role in NK lineage differentiation and no NK cells would grow out from bone marrow or thymocytes cultured on OP9-DL1 stromal cells. Consistent with the idea that Bcl11b acts downstream of Notch signalling in T cells, once Bcl11b was deleted, iTNK production from T cells was independent of Notch signalling because T to NK conversion occurred using either OP9 or OP9-DL1 stromal cells (data not shown).

To confirm that Bcl11b is directly regulated by Notch signalling in mouse T cells at the molecular level, we first searched within the Bcl11b gene locus for putative CSL-binding sites (CGTGGGAA) (26) at the Bcl11b locus, which were conserved between mouse and human Bcl11b genes (FIG. 4B) (table 4). Several CSL sites were identified but we focused our attention on the ones that were conserved between mouse and human Bcl11b genes. Chromatin immunoprecipitation (ChIP) assay was subsequently performed using a CSL polyclonal antibody pulled down genomic DNA fragments from T cells. Three genomic regions were greatly enriched in the T cell samples using the CSL antibody compared to the control (FIG. 4C). Primers flanking the putative CSL binding regions were designed to amplify the ChIP pull-down genomic DNA (FIG. 4B). Regions 3, 4, 7 were greatly enriched in the T cell samples using the CSL antibody compared to using the antibody control (FIG. 4C and Table 4). Region 3 is about 1.8 kb from start of the transcription. Region 4 was located 5.4 kb downstream of exon 1; and region 7 was at about 600 bp downstream of exon 2 The ChIP result thus confirmed that the canonical Notch signaling directly regulated Bcl11b in T cells (FIG. 12).

However, it is reported that deleting CSL (RBPJk) using either CD4-Cre or Lck-Cre did not cause total T cell loss or lead to production of ITNKs (38). This discrepancy likely reflects that we acutely deleted Bcl11b in T cells for immediate functional consequences whereas if CD4-Cre is used, the deletion can occur in progenitors. Consequently, in CD4-Cre mice, the cells having defects are those from mutant progenitors and have developed mechanisms to compensate for the loss of Bcl11b. We propose that Bcl11b is a downstream target gene of Notch signalling, and that Bcl11b, together with other Notch downstream transcription factors Gata3 and Tcf1, play pivotal roles in specification, commitment and maintenance of the T cell lineage.

We show that Bcl11b was essential for T cell development and maintenance of T cell identity. Unlike loss of Pax5 in B cells (39), however, deletion of Bcl11b did not appear to have detectable de-differentiation steps because both lymphocytes and mature T cells were readily reprogrammed to ITNKs, and ITNKs from DP thymocytes and mature T cells still retained expression of TCRβ, CD4 or CD8. This “transdifferentiation” might reflect the fact that T and NK lineages were diverted late in hematopoiesis and thus loss of one transcription factor, Bcl11b, was sufficient to cause lineage switch with 100% efficiency.

Because ITNKs reprogrammed from mature T cells retain TCRβ expression, it is possible that Bcl11b mainly functions as a suppressor of NK lineage rather than promoting and maintaining the T cell linage. Our data however do not support this possibility: ITNKs are different from NK cells, even those reprogrammed from DN1-DN2 thymocytes; Bcl11b is expressed at certain stages of NK development; Although ITNKs from mature T cells retain more T cell properties, they are still vastly different from either T cells or NK cells, and have no or diminished expression of IL7Ra, CD4, CD8, CD3 and T-bet (FIG. 10G); Microarray data show that in OHT-treated thymocytes (Bcl11b deletion), in the first 24 hours, down-regulation of T cell-associated genes account for almost all the gene expression changes. NK-associated genes expression follows down-regulation of T cell genes and starts after 48 hours following Bcl11b deletion. Master regulators that promote a cell lineage and that are required to maintain lineage identity have been identified for several cell lineages. For example, ectopically expressing Cebpa in pro-B and pro-T cells transforms them into macrophages at a frequency of around 60% (Laiosa et al., 2006; Xie et al., 2004). 25-50% of fibroblast cells expressing MyoD convert to myogenic colonies (Davis et al., 1987). Recently, it is shown that pancreatic acinar cells expressing three TFs, Pdx1, Ngn3 and Mafa is able to convert them into insulin-expressing β cells in vivo at an estimated frequency of 20%. Additionally, loss of Pax5 in B cells enables de-differentiation of B cells to become multi-potent progenitors (Mikkola et al., 2002). Similar to Pax5 in B cells, we show here that Bcl11b is essential for T cell development and currently the only known transcription factor for T cell identity maintenance. However, unlike de-differentiation in B cell upon loss of Pax5 (Cobaleda et al., 2007), deletion of Bcl11b in T cells does not appear to have obvious or prolonged de-differentiation steps because both pro-T and mature T cells readily convert to ITNKs. Moreover, ITNKs from DP thymocytes and mature T cells still retained Tcrβ expression. This may reflect the fact that T and NK lineages are diverted late during T cell development in the thymus and thus loss of one transcription factor, Bcl11b, is sufficient to convert T cells into iTNK cells with 100% efficiency. Our study therefore adds Bcl11b to the collection of transcription factors that play pivotal roles in hematopoietic lineage specification, commitment and maintenance.

NK cell-based therapies hold promise in cancer treatment. We are now able to reprogramme T cells to ITNKs, which can be extensively expanded but are not malignantly transformed. Rather, they effectively killed tumour cells in vitro and eliminated metastatic cells in mice but did not appear to attack normal cells. Therefore, ITNK cells may serve as a new cell source for cancer immunotherapy and other cell-based therapies.

### REFERENCES

1. M. Ciofani, J. C. Zuniga-Pflucker, Annu Rev Cell Dev Biol 23, 463 (2007).
2. E. V. Rothenberg, J. E. Moore, M. A. Yui, Nat Rev Immunol 8, 9 (January, 2008).
3. K. Takada, S. C. Jameson, Nat Rev Immunol 9, 823 (December, 2009).
4. C. C. Tydell et al., J Immunol 179, 421 (Jul. 1, 2007).
5. B. Bajoghli et al., Cell 138, 186 (Jul. 10, 2009).
6. Y. Wakabayashi et al., Nat Immunol 4, 533 (June, 2003).
7. D. I. Albu et al., J Exp Med 204, 3003 (Nov. 26, 2007).
8. C. A. Vosshenrich et al., Nat Immunol 7, 1217 (November, 2006).
9. D. Hameyer et al., Physiol Genomics 31, 32 (Sep. 19, 2007).
10. T. M. Schmitt, J. C. Zuniga-Pflucker, Immunity 17, 749 (December, 2002).
11. T. M. Schmitt, M. Ciofani, H. T. Petrie, J. C. Zuniga-Pflucker, J Exp Med 200, 469 (Aug. 16, 2004).
12. T. Walzer et al., Proc Natl Acad Sci USA 104, 3384 (Feb. 27, 2007).
13. M. D. Boos, K. Ramirez, B. L. Kee, Immunol Res 40, 193 (2008).
14. S. M. Chambers et al., Cell Stem Cell 1, 578 (November, 2007).
15. D. M. Gascoyne et al., Nat Immunol (Sep. 13, 2009).
16. S. C. Miaw, A. Choi, E. Yu, H. Kishikawa, I. C. Ho, Immunity 12, 323 (March, 2000).
17. A. Topark-Ngarm et al., J Biol Chem 281, 32272 (Oct. 27, 2006).
18. Y. Kawano, K. Taniguchi, A. Toshitani, K. Nomoto, J Immunol 136, 4729 (Jun. 15, 1986).
19. K. Karre, H. G. Ljunggren, G. Piontek, R. Kiessling, Nature 319, 675 (Feb. 20-26, 1986).
20. H. G. Ljunggren, K. Karre, J Exp Med 162, 1745 (Dec. 1, 1985).
21. F. Colucci et al., J Immunol 162, 2761 (Mar. 1, 1999).
22. E. Gorelik, R. H. Wiltrout, K. Okumura, S. Habu, R. B. Herberman, Int J Cancer 30, 107 (Jul. 15, 1982).
23. T. Lakshmikanth et al., J Clin Invest 119, 1251 (May, 2009).
24. E. V. Rothenberg, Nat Immunol 8, 441 (May, 2007).
25. A. Krejci, F. Bernard, B. E. Housden, S. Collins, S. J. Bray, Sci Signal 2, ra1 (2009).
26. T. Tun et al., Nucleic Acids Res 22, 965 (Mar. 25, 1994).
27. E. C. Forsberg, K. M. Downs, E. H. Bresnick, Blood 96, 334 (Jul. 1, 2000).
28. G. Anderson, N. C. Moore, J. J. Owen, E. J. Jenkinson, Annu Rev Immunol 14, 73 (1996).
29. D. I. Godfrey, J. Kennedy, T. Suda, A. Zlotnik, J Immunol 150, 4244 (May 15, 1993).
30. H. Wada et al., Nature 452, 768 (Apr. 10, 2008).
31. J. J. Bell, A. Bhandoola, Nature 452, 764 (Apr. 10, 2008).
32. E. C. Dudley, H. T. Petrie, L. M. Shah, M. J. Owen, A. C. Heyday, Immunity 1, 83 (May, 1994).
33. H. T. Petrie, P. Hugo, R. Scollay, K. Shortman, J Exp Med 172, 1583 (Dec. 1, 1990).
34. J. Nikolic-Zugic, M. W. Moore, M. J. Bevan, Eur J Immunol 19, 649 (April, 1989).
35. J. P. Di Santo, Annu Rev Immunol 24, 257 (2006).
36. N. D. Huntington, C. A. Vosshenrich, J. P. Di Santo, Nat Rev Immunol 7, 703 (September, 2007).
37. J. C. Sun, J. N. Beilke, L. L. Lanier, Nature 457, 557 (Jan. 29, 2009).
38. K. Tanigaki et al., Immunity 20, 611 (May, 2004).
39. C. Cobaleda, W. Jochum, M. Busslinger, Nature 449, 473 (Sep. 27, 2007).
40. Allman, D., Sambandam, A., Kim, S., Miller, J. P., Pagan, A., Well, D., Meraz, A., and Bhandoola, A. (2003). Thymopoiesis independent of common lymphoid progenitors. Nature immunology 4, 168-174.
41. Amsen, D., Antov, A., and Flavell, R. A. (2009). The different faces of Notch in T-helper-cell differentiation. Nature reviews 9, 116-124.
42. Avram, D., Fields, A., Pretty On Top, K., Nevrivy, D. J., Ishmael, J. E., and Leid, M. (2000). Isolation of a novel family of C(2)H(2) zinc finger proteins implicated in transcriptional repression mediated by chicken ovalbumin upstream promoter transcription factor (COUP-TF) orphan nuclear receptors. The Journal of biological chemistry 275, 10315-10322.
43. Barton, K., Muthusamy, N., Fischer, C., Ting, C. N., Walunas, T. L., Lanier, L. L., and Leiden, J. M. (1998). The Ets-1 transcription factor is required for the development of natural killer cells in mice. Immunity 9, 555-563.
44. Bendelac, A., Savage, P. B., and Teyton, L. (2007). The biology of NKT cells. Annual review of immunology 25, 297-336.
45. Benne, C., Lelievre, J. D., Balbo, M., Henry, A., Sakano, S., and Levy, Y. (2009). Notch Increases T/NK Potential Of Human Hematopoietic Progenitors and Inhibits B Cell Differentiation At A Pro-B stage. Stem cells (Dayton, Ohio).
46. Blom, B., Heemskerk, M. H., Verschuren, M. C., van Dongen, J. J., Stegmann, A. P., Bakker, A. Q., Couwenberg, F., Res, P. C., and Spits, H. (1999). Disruption of alpha beta but not of gamma delta T cell development by overexpression of the helix-loop-helix protein Id3 in committed T cell progenitors. Embo J 18, 2793-2802.
47. Carotta, S., Brady, J., Wu, L., and Nutt, S. L. (2006). Transient Notch signaling induces NK cell potential in Pax5-deficient pro-B cells. European journal of immunology 36, 3294-3304.
48. Cismasiu, V. B., Adamo, K., Gecewicz, J., Duque, J., Lin, Q., and Avram, D. (2005). BCL11B functionally associates with the NuRD complex in T lymphocytes to repress targeted promoter. Oncogene 24, 6753-6764.
49. Davis, R. L., Weintraub, H., and Lassar, A. B. (1987). Expression of a single transfected cDNA converts fibroblasts to myoblasts. Cell 51, 987-1000.
50. De Smedt, M., Hoebeke, I., Reynvoet, K., Leclercq, G., and Plum, J. (2005). Different thresholds of Notch signaling bias human precursor cells toward B-, NK-, monocytic/dendritic-, or T-cell lineage in thymus microenvironment. Blood 106, 3498-3506.
51. DeHart, S. L., Heikens, M. J., and Tsai, S. (2005). Jagged2 promotes the development of natural killer cells and the establishment of functional natural killer cell lines. Blood 105, 3521-3527.
52. Feyerabend, T. B., Terszowski, G., Tietz, A., Blum, C., Luche, H., Gossler, A., Gale, N. W., Radtke, F., Fehling, H. J., and Rodewald, H. R. (2009). Deletion of Notch1 converts pro-T cells to dendritic cells and promotes thymic B cells by cell-extrinsic and cell-intrinsic mechanisms. Immunity 30, 67-79.
53. Fowlkes, B. J., and Robey, E. A. (2002). A reassessment of the effect of activated Notch1 on CD4 and CD8 T cell development. J Immunol 169, 1817-1821.
54. Franksson, L., George, E., Powis, S., Butcher, G., Howard, J., and Karre, K. (1993). Tumorigenicity conferred to lymphoma mutant by major histocompatibility complex-encoded transporter gene. The Journal of experimental medicine 177, 201-205.
55. Fujimoto, S., Ikawa, T., Kina, T., and Yokota, Y. (2007). Forced expression of Id2 in fetal thymic T cell progenitors allows some of their progeny to adopt NK cell fate. Int Immunol 19, 1175-1182.
56. Garcia-Peydro, M., de Yebenes, V. G., and Toribio, M. L. (2006). Notch1 and IL-7 receptor interplay maintains proliferation of human thymic progenitors while suppressing non-T cell fates. J Immunol 177, 3711-3720.
57. Grabarczyk, P., Przybylski, G. K., Depke, M., Volker, U., Bahr, J., Assmus, K., Broker, B. M., Walther, R., and Schmidt, C. A. (2007). Inhibition of BCL11B expression leads to apoptosis of malignant but not normal mature T cells. Oncogene 26, 3797-3810.
58. Guo, Y., Maillard, I., Chakraborti, S., Rothenberg, E. V., and Speck, N. A. (2008). Core binding factors are necessary for natural killer cell development and cooperate with Notch signaling during T-cell specification. Blood 112, 480-492.
59. Haraguchi, K., Suzuki, T., Koyama, N., Kumano, K., Nakahara, F., Matsumoto, A., Yokoyama, Y., Sakata-Yanagimoto, M., Masuda, S., Takahashi, T., et al. (2009). Notch activation induces the generation of functional NK cells from human cord blood CD34-positive cells devoid of IL-15. J Immunol 182, 6168-6178.
60. Herberman, R. B., and Ortaldo, J. R. (1981). Natural killer cells: their roles in defenses against disease. Science (New York, N.Y. 214, 24-30.
61. Ho, I. C., Tai, T. S., and Pai, S. Y. (2009). GATA3 and the T-cell lineage: essential functions before and after T-helper-2-cell differentiation. Nature reviews 9, 125-135.
62. Ikawa, T., Fujimoto, S., Kawamoto, H., Katsura, Y., and Yokota, Y. (2001). Commitment to natural killer cells requires the helix-loop-helix inhibitor Id2. Proceedings of the National Academy of Sciences of the United States of America 98, 5164-5169.
63. Ikawa, T., Kawamoto, H., Fujimoto, S., and Katsura, Y. (1999). Commitment of common T/Natural killer (NK) progenitors to unipotent T and NK progenitors in the murine fetal thymus revealed by a single progenitor assay. The Journal of experimental medicine 190, 1617-1626.
64. Kang, B. Y., Miaw, S. C., and Ho, I. C. (2005). ROG negatively regulates T-cell activation but is dispensable for Th-cell differentiation. Molecular and cellular biology 25, 554-562.
65. Laiosa, C. V., Stadtfeld, M., Xie, H., de Andres-Aguayo, L., and Graf, T. (2006). Reprogramming of committed T cell progenitors to macrophages and dendritic cells by C/EBP alpha and PU.1 transcription factors. Immunity 25, 731-744.
66. Maillard, I., Fang, T., and Pear, W. S. (2005). Regulation of lymphoid development, differentiation, and function by the Notch pathway. Annual review of immunology 23, 945-974.
67. Michie, A. M., Carlyle, J. R., Schmitt, T. M., Ljutic, B., Cho, S. K., Fong, Q., and Zuniga-Pflucker, J. C. (2000). Clonal characterization of a bipotent T cell and NK cell progenitor in the mouse fetal thymus. J Immunol 164, 1730-1733.
68. Mikkola, I., Heavey, B., Horcher, M., and Busslinger, M. (2002). Reversion of B cell commitment upon loss of Pax5 expression. Science (New York, N.Y. 297, 110-113.
69. Porritt, H. E., Rumfelt, L. L., Tabrizifard, S., Schmitt, T. M., Zuniga-Pflucker, J. C., and Petrie, H. T. (2004). Heterogeneity among DN1 prothymocytes reveals multiple progenitors with different capacities to generate T cell and non-T cell lineages. Immunity 20, 735-745.
70. Radtke, F., Wilson, A., Stark, G., Bauer, M., van Meerwijk, J., MacDonald, H. R., and Aguet, M. (1999). Deficient T cell fate specification in mice with an induced inactivation of Notch1. Immunity 10, 547-558.
71. Rolink, A. G., Balciunaite, G., Demoliere, C., and Ceredig, R. (2006). The potential involvement of Notch signaling in NK cell development. Immunol Lett 107, 50-57.
72. Samson, S. I., Richard, O., Tavian, M., Ranson, T., Vosshenrich, C. A., Colucci, F., Buer, J., Grosveld, F., Godin, I., and Di Santo, J. P. (2003). GATA-3 promotes maturation, IFN-gamma production, and liver-specific homing of NK cells. Immunity 19, 701-711.
73. Shortman, K., and Wu, L. (1996). Early T lymphocyte progenitors. Annual review of immunology 14, 29-47.
74. Taghon, T., Yui, M. A., and Rothenberg, E. V. (2007). Mast cell lineage diversion of T lineage precursors by the essential T cell transcription factor GATA-3. Nature immunology 8, 845-855.
75. Townsend, M. J., Weinmann, A. S., Matsuda, J. L., Salomon, R., Farnham, P. J., Biron, C. A., Gapin, L., and Glimcher, L. H. (2004). T-bet regulates the terminal maturation and homeostasis of NK and Valphal41 NKT cells. Immunity 20, 477-494.
76. Tun, T., Hamaguchi, Y., Matsunami, N., Furukawa, T., Honjo, T., and Kawaichi, M. (1994). Recognition sequence of a highly conserved DNA binding protein RBP-J kappa. Nucleic acids research 22, 965-971.
77. van den Brandt, J., Voss, K., Schott, M., Hunig, T., Wolfe, M. S., and Reichardt, H. M. (2004). Inhibition of Notch signaling biases rat thymocyte development towards the NK cell lineage. European journal of immunology 34, 1405-1413.
78. Vivier, E., Tomasello, E., Baratin, M., Walzer, T., and Ugolini, S. (2008). Functions of natural killer
79. Washburn, T., Schweighoffer, E., Gridley, T., Chang, D., Fowlkes, B. J., Cado, D., and Robey, E. (1997). Notch activity influences the alphabeta versus gammadelta T cell lineage decision. Cell 88, 833-843.
80. Wolfer, A., Wilson, A., Nemir, M., MacDonald, H. R., and Radtke, F. (2002). Inactivation of Notch1 impairs VDJbeta rearrangement and allows pre-TCR-independent survival of early alpha beta Lineage Thymocytes. Immunity 16, 869-879.
81. Xie, H., Ye, M., Feng, R., and Graf, T. (2004). Stepwise reprogramming of B cells into macrophages. Cell 117, 663-676.
82. Yokota, Y., Mansouri, A., Mori, S., Sugawara, S., Adachi, S., Nishikawa, S., and Gruss, P. (1999). Development of peripheral lymphoid organs and natural killer cells depends on the helix-loop-helix inhibitor Id2. Nature 397, 702-706.

### FIGURE LEGENDS

FIG. 1. Bcl11b is essential for T cell development and for maintaining T cell identity. Thymocytes from flox/flox or flox/+ control mice were treated, or not, with OHT then sorted into DN1 or DN2 subsets, and cultured on OP9-DL1 stromal cells. (A) Flow cytometry profiles of cultured DN1 and DN2 thymocytes (+OHT) in the absence of IL-2. Numbers refer to percentage of cells in the gate. Data are representative of three experiments. (B) Flow cytometry profiles of cultured flox/flox DN3 thymocytes (±OHT) supplemented with IL-2. Data are representative of three experiments. Bcl11b-deficient DN3 thymocytes lost T cell identity and converted to NKp46 expressing cells. −OHT: non-treated cells; +OHT: treated cells. (C) Killing of OP9-DLI stromal cells by OHT-treated flox/flox DN3 thymocytes. Scale bar: 40 μm. The NKp46⁺ cells from Bcl11b deficient DN3 thymocytes (+OHT) killed OP9-DL1 stromal cells effectively. (D) DNA from purified NKp46⁺ cells was prepared and subjected to PCR to detect DJ (top) and VDJ (bottom) recombination at the TCRβ locus. T: T cells growing from untreated DN3 thymocytes; N1 and N2: sorted NKp46⁺ cells growing from OHT-treated flox/flox DN3 thymocytes; Thy: wild type whole thymocytes; B: B cells; GL: germline band; H₂O: no DNA template in PCR. Numbers indicate DJ recombination products. The NKp46⁺ cells from Bcl11b deficient DN3 thymocytes still retained V(D)J recombination at the Tcrβ locus even though they did not express Tcrβ. (E-G) Microarray analysis of gene expression in NKp46⁺CD3⁺ ITNK cells from DN3 thymocytes (I1-I4), IL-2-expanded NK cells (LAK; L1-L4) and sorted DN3 flox/flox thymocytes (DN3; D1-D4) were subjected to expression. (E) Two-way hierarchical cluster map of the array data. Column numbers (I1-I4 for instance) refer to 4 independent RNA samples for each cell type and rows represent individual transcripts. Scale indicates the log2 value of normalized signal level. Comparison of expression profiles of parental DN3 thymocytes, iTNK cells derived from DN3 thymocytes and regular NK cells (LAKs). RNA samples were made from 4 mice for each cell type. (F) qRT-PCR validation of gene expression of selected genes among ITNKs, LAKs and DN3 cells. Bars are mean+SD of 3 samples. In each histogram in FIG. 1 (F), the first bar represents DN3 cells, the second bar represents ITNKs and the third bar represents LAKs. (G) qRT-PCR validation of gene expression difference among DN3, iTNK and LAK cells. Expression of T cell specific genes was generally decreased, and expression of NK-specific genes was greatly increased in the NK-like cells. Zbtb23 (Rog) and Cdkn1c (p57Kip) were not normally expressed in DN3 thymocytes. In each histogram in FIG. 1 (G), the first bar represents LAK cells, the second bar represents ITNKs and the third bar represents DN3 cells.

FIG. 2. Efficient reprogramming of T cells to ITNKs. (A) Representative flow cytometry profiles of ITNKs reprogrammed from single flox/flox DN3 cells. Numbers refer to percentage in total cells. T: T cells that did not have complete Bcl11b deletion. Data are representative of three experiments. NKp46⁺ iTNKs derived from single Bcl11b-deficient DN3 thymocytes in individual wells (96-well plate) co-cultured with OP9-DL1 stromal cells. T: cells that expressed T cell genes and Bcl11b was not completely deleted; iTNK: cells that had deleted both copies of Bcl11b and expressed NKp46.

(B) PCR genotyping of Bcl11b deletion in two representative T cell (T1, T2) and ITNK (I1, I2) wells. flox: floxed allele; del; deletion allele. −OHT: no OHT treatment; H₂O: no template control. PCR-genotyping indicated that cells in some wells did not have complete Cre-loxP recombination (T1 and T2). These cells had one deletion allele and one cko allele at the Bcl11b locus. On the other hand, all the NKp46⁺ cells had Bcl11b completely deleted (11 and 12). No deletion was detected in cells without OHT treatment (−OHT).

(C) DJ recombination at the TCRβ locus of five ITNK wells (I1-I5) showing unique DJ recombination. L: DNA ladder; Thy: wild type thymocytes. (D) Giemsa stain of parental DN3 thymocytes (T) and ITNK cells. Scale bar: 20 μm. (E) Transmission electron micrograph of an ITNK cell. 1: Nucleus; 2. Golgi body; 3. Granule; 4. ER. Scale bar: 2 μm. (e) Electron Transmission Microscopy image of ITNK cells shows prominent Golgi and ERs, and granules. Arrows: 1=nucleus; 2=ER; 3=granule; 4=golgi. (F) Cytotoxicity of ITNKs (labeled as “+OHT”) and LAKs measured in standard ⁵¹Cr release assays with B16F10, RMA and RMA-S tumor cell targets at the indicated effector-to-target (E:T) ratios. −OHT: flox/flox T cells. Data are mean of triplicate wells. In vitro derived ITNK cells from DN3 thymocytes killed tumour cells effectively. Both LAK and ITNK cells killed MHC—I negative B16F10 melanoma and RMA-S lymphoma cells.

FIG. 3. ITNKs reprogrammed in vivo were potent tumour cell killers. (A) Flow cytometric analysis of thymocytes and splenocytes from OHT treated flox/flox and flox/+ mice. Numbers refer to percentage in lymphocyte gate. Data are representative of four mice. (B) Analysis of ITNKs from thymic γδ cells in OHT treated flox/flox mice. Data are representative of two mice. (C) ITNKs production in Rag2^{-/-}Il2rg^{-/-} recipients injected with flox/flox DP thymocytes. Two weeks after injection, donor (CD45.2⁺) and host (CD45.1⁺) splenocytes were analyzed. Numbers refer to percentage of lymphocyte gate. Plots are representative of 15 mice from three independent experiments. Donor cells were identified by CD45.2 staining. About 5% of splenocytes were donor derived and roughly half of these donor-derived cells were NKp46⁺ iTNKs. (D) Ex vivo expansion of ITNKs in IL-2 from splenocytes of the recipient mice. Viable cells were counted and analyzed (bottom panel) at the indicated time points. Numbers refer to percentages. Most cells in the culture were ITNKs because they expressed NKp46, TCRβ, NK1.1 and NKG2D. Bars are mean±SD of 4 samples. Data are representative of three experiments. (d) Ex vivo expansion of in vivo reprogrammed iTNK cells starting from splenotypes of four Rag2^{-/-}Il2γc^{-/-} recipient mice. These cells were able to proliferate extensively in the culture for up to 3-4 weeks. Bottom panel: iTNK cells (NK1.1⁺ and/or NKp46⁺) accounted for the majority of the cells in the culture after one week culturing. (E) The ex vivo-expanded ITNKs (labeled as “+OHT”) were used in ⁵¹Cr release killing assays with B16F10, RMA and RMA-S tumor cell targets at the indicated effector-to-target (E:T) ratios. −OHT: flox/flox T cells. Data are mean of triplicate wells. Results are representative of three experiments. Ex vivo expanded iTNKs were more potent killers for tumour cells than LAKs. iTNKs effectively killed tumour cells of either MHC—I positive or negative.

(F) ITNKs prevented tumour metastasis. Rag2^{-/-}Il2rg^{-/-} recipients first transplanted with treated (+OHT) or untreated (−OHT) flox/flox DP thymocytes or PBS. Recipients were subsequently injected intravenously with 50,000 B16F10 melanoma cells. Lung tumour colonies were enumerated two weeks after tumour challenge. Data are from individual mice and bar represents the mean. (G) In vivo iTNKs effectively eliminated B16F10 melanoma cells in mice. Many metastatic colonies were visible in the lung of the control mice that were injected with either PBS (no cells) or untreated DP thymocytes (−OHT). Very few metastatic colonies existed if OHT-treated DP thymocytes were injected and hence iTNK were produced (+OHT).

FIG. 4. Bcl11b is a direct downstream target gene of Notch signaling. (A). Bcl11b protein in T cells following OHT treatment detected by Western blot. (B) Schematic of the Bcl11b locus showing putative CSL binding sites (BS) and that of an irrelevant control binding site (CTL). (C) Genomic DNA was prepared from immunoprecipitation of thymocytes, using CSL or control IgG antibodies, and was amplified using primers flanking the putative CSL or the control binding sites at the Bcl11b locus. Three Bcl11b binding regions: Region 1, about 1.8 kb from start of the transcription; Region 2, 5.4 kb downstream of exon 1; region 3, about 600 bp downstream of exon 2. CSL: CSL antibody; IgG: control IgG. Fold-enrichment was calculated relative to the IgG control (set to 1). Bars are mean±SD of triplicate. In the histogram in FIG. 4 (c), the first bar represents CSL and the second bar represents IgG.

FIG. 5. Generation of the Bcl11b-tdTomato reporter mouse. (A) The tdTomato cassette was targeted to the 3′ UTR of the Bcl11b locus. (B) Insertion of the tdTomato cassette at the Bcl11b 3′ UTR did not affect T cell development. Numbers refer to percentage of lymphocytes gate. Data are representative of three mice.

FIG. 6. Detection of Bcl11b expression in hematopoietic lineages using the Bcl11b-tdTomato reporter mice. Leukocytes from the thymus, spleen and bone marrow of Bcl11b^{fd/+} mice were labeled with antibodies for flow cytometric analysis. Bcl11b-expressing cells had red fluorescence. Solid line refers to Bcl11b^{fd/+} mice and dashed line refers to wild type mouse. (A) CD4⁻CD8⁻ double negative (DN; DN1-DN4) thymocyte subsets. DN1: CD44⁺CD25⁻; DN2: CD44⁺CD25⁺; DN3: CD44⁻CD25⁺; DN4: CD44⁻CD25⁻. (B) Double positive (DP) thymocytes (CD4⁺CD8⁺), splenic CD4⁺ and CD8⁺ T cells, thymic γδ T cells, and splenic NKT cells (CD3⁺CD1d⁺). (C) Bone marrow B cells (CD19⁺B220⁺) and myeloid cells (CD11b⁺Gr-1⁺). (D) Splenic (CD3⁻), and thymic (CD3⁻CD4⁻CD8⁻) NK cells. NKP: NK cell precursor; Immature: NK1.1⁺CD27⁺CD11b⁻ and NK1.1⁺CD27⁺CD11b⁺. (E) qRT-PCR of Bcl11b expression in sorted splenic naïve (CD44CD62⁻L⁺) and activated (CD44⁺CD62L⁻) T cells population. Bcl11b expression was calculated relative to that in CD8⁺CD44⁺CD62L⁻ (set to 1). Bars are mean±SEM of 3 samples. (F) Quantification of Bcl11b expression in naïve and activated T cells in the Bcl11b^{fd/+} mice. Percentages refer to the indicated T cell subsets in Bcl11b^{fd/+} mice. All FACS data in this figure are representative of three experiments.

FIG. 7. Strategies for identification of cell populations for flow sorting and analysis. (A) Identification of double negative (DN) thymocyte (DN1-DN4) populations defined by Lin⁻ and expression of CD25 and CD44. DN1 subpopulations were defined by expression of CD117 (c-Kit). Numbers refer to percentages. (B) Identification of γδ T cells. (C) Identification of NKT cells in the spleen by first gating (or, prior to FACS sorting, magnetically depleting) out B cells. INKTs were CD3⁺ and stained positively by CD1d dimer. (D) Identification of NK precursors (CD3⁻CD122⁺NK1.1⁻) and NK cell subsets (NK1.1⁺CD27⁺CD11 b⁻, NK1.1⁺CD27⁺CD11b⁺, NK1.1⁺CD27⁻CD11b⁺) cells. (E) Thymic NK cells were defined as NK1.1⁺CD127⁺ thymocytes. (F) Identification of naïve (CD44⁻CD62L⁺) and activated (CD44⁺CD62L⁻) T cells.

FIG. 8. In vitro analysis of Bcl11b-deficient T cells. (A) Schematic diagram of the Bcl11b conditional knockout allele. Bcl11b exon 4 was flanked by loxP sites. Indicated DNA fragments were detected by the 5′ probe in Southern blot analysis of targeted ES cells. Southern blot analysis of the targeted ES cell clones using a 5′ probe which detected a 27 kb wild type BamHI band. The same probe hybridized to a 12.6 kb fragment in the conditional knockout clones (cko/+) and a 17.5 kb fragment in clones that did not have the 5′ IoxP site (+/−). (B) Experimental design for the analysis of Bcl11b-deficient DN thymocytes. Whole thymocytes from CreERT2; Bcl11b^{flox/flox} (fox/fox) or CreERT2; Bcl11b^{flox/+} (flox/+) mice were treated with OHT (+OHT) or left untreated (−OHT) for 48 hr then sorted into the indicated subset and cultured on OP9-DL1 stromal cells for 2 weeks. (C) NKp46⁺CD3⁻ cells from DN1 and DN20HT-treated flox/flox thymocytes did not express TCRβ. Numbers refer to percentage of cells. Data are representative of two experiments. (D) Homozygous Bcl11b deletion in ITNK (NKp46⁺CD3⁻) but not in T (NKp46⁻CD3⁺) cell populations from DN1 and DN2 cultures. flox: conditional knockout allele; del: deletion allele. H₂O: no DNA template control. (E) No NKp46⁺ cells but T cells were obtained from untreated flox/flox thymocytes. (F) NKp46⁺TCRβ⁻ cells from OHT-treated DN1 and DN2 flox/flox thymocytes in the absence of IL-2 or IL-15 cultured on OP9 stromal cells. (G) NKp46⁺TCRβ⁻ cells were detected in OHT-treated DN3 flox/flox, but not flox/+, thymocytes in T cell media. (H) Reprogramming of Bcl11b-deficient DN3 thymocytes to NKp46⁺ cells in myeloid cell culture condition. (I) Reprogramming of Bcl11b-deficient DN3 thymocytes to NKp46⁺CD19⁻ cells in B cell culture condition. (J) Venn diagram comparison of the upregulated (>2-fold) genes between LAK vs DN3 (green) and ITNK vs DN3 (purple) shows a significant overlapping between the two gene lists. (K) ITNKs from DP flox/flox thymocytes treated with OHT and cultured on OP9-DL1 in the presence of IL-2. Untreated cells died rapidly under this condition. (L) ITNKs from splenic flox/flox CD8⁺ T cells treated with OHT cultured on OP9-DL1 in the presence of IL-2. All FACS data in this figure are representative of 2-4 experiments.

FIG. 9. Characterization of in vitro reprogrammed ITNK phenotype. (A) Experimental design for reprogramming of single DN3 thymocytes to ITNK. Whole thymocytes from flox/flox mice were treated with OHT (+OHT) or left untreated (−OHT) and 48-hours later single DN3 cells were sorted and seeded on OP9-DL1 stromal cells in 96-well plates for 10-14 days supplemented with IL-2. (a) Experimental design for analyzing single DN3 thymocytes conversion to iTNKs. DN3 thymocytes (either treated with OHT, or untreated) were sorted into individual wells of 96-well plates pre-seeded with OP9-DL1 stromal cells. Two weeks (with 112) or three weeks (without 112) later, the OHT-treated DN3 cells (Bcl11b-deficient) converted to iTNKs, confirmed by FACS analysis and genomic DNA PCR. (B-C) Expression of intracellular (TRAIL, perforin, IFNγ) and NK cell surface markers by the reprogrammed ITNK from DN3 thymocytes in vitro. (D) Expression of NK cell markers by ITNKs reprogrammed from Bcl11b-deficient DP thymocytes in vitro. (E) ITNKs did not express CD127 and thus were not thymic NK cells. (F) Analysis of CD27 and CD11b in bulk-cultured ITNKs reprogrammed from DN3 thymocytes. All FACS data are representative of three experiments.

FIG. 10. Analysis of in vivo reprogrammed ITNK cells in the flox/flox mouse. (A) Experimental design for the analysis of in vivo reprogrammed ITNK cells. flox/flox or flox/+ mice were treated with Tamoxifen by oral gavage on three consecutive days, and the thymi and spleens were analyzed 2-3 weeks later. We observed a 5-10 fold reduction in total thymocytes and about 2-fold reduction in splenocytes in the treated flox/flox mice compared to treated flox/+ control mice. (B) PCR of Bcl11b deletion in ITNK (NKp46⁺CD3⁺ and NKp46⁺CD3⁻) cell populations in flox/flox mice. flox: conditional knockout allele; del: deletion allele. H₂O: no DNA template control. All the NKp46⁺CD3⁺ and NKp46⁺CD3⁻ cells in the thymus were ITNKs. Analyzing ITNKs in the spleen was more complicated due to the presence of many NKp46⁺ conventional NK cells. However, most of the NKp46⁺CD3⁺ cells in the spleen had Bcl11b deficiency and thus were ITNKs. PCR data are representative of three experiments. (C) Flow cytometric analysis of CD4 and CD8 expression in NKp46⁺ ITNKs. Note that both CD4 and CD8 expression was down in ITNKs (CD4⁺NKp46⁺ or CD8⁺NKp46⁺) compared to CD4⁺NKp46⁻ or CD8⁺NKp46⁻ T cells. (D) Flow cytometric analysis of cells following ex vivo expansion of whole thymocytes or splenocytes from OHT treated mice. (E) Flow cytometric analysis of CD1d-restriced NKT cells in thymus and spleen. Total lymphocytes and CD19⁻ splenocytes were gated in the thymus and spleen, respectively. Note the reduction of NKT cells in the OHT-treated flox/flox mice. (F) Analysis of CD1d-restricted cells in the ex vivo-expanded ITNK culture. Numbers refer to percentages in lymphocyte gate. All FACS data in this figure are representative of 3-4 individual mice. (G) qRT-PCR analysis of several key T or NK cell-associated genes in CD8⁺ T cells, CD8⁺ ITNKs and LAKs. Bars are mean±SEM of 3 samples. The highest expression level for each gene was chosen as 1. (H) Splenocytes from flox/flox or flox/+ mice treated with Tamoxifen were stained with NKp46, NK1.1, CD8 and CD3 to confirm expression of CD3 on ITNKs. A separate aliquot was loaded with Indo-1, stained with antibodies to NKp46, NK1.1 and CD8 and analyzed for calcium flux by flow cytometry. Top panel: Phenotype of splenocytes from flox/flox or flox/+ mice indicating gated T cells (CD3⁺NKp46⁻) and ITNKs (CD3⁺NKp46⁺) cells. Numbers refer to percentages in gates of total lymphocytes. Lower panel: Calcium flux plots from the indicated cell subset. A baseline was established at the start of the assay, before acquisition was interrupted and anti-CD3 (145-2C11) was added (first arrow). CD3 was then cross-linked by addition of anti-hamster secondary antibody (second arrow). Ionomycin was added (third arrow) as a positive control. Numbers in gates refer to responders (upper gate) and non-responders (lower gates) after addition of anti-hamster antibody. Data below calcium plots show ratio of responders to non-responders. Data are representative of two mice.

FIG. 11. In vivo reprogrammed ITNKs from DP thymocytes prevented tumour metastasis. (A) Experimental design for the analysis of in vivo reprogramming of DP thymocytes to ITNKs. Whole thymocytes from flox/flox mice were treated with OHT (+OHT) or left untreated (−OHT) and 48-hours later DP cells were sorted and injected intravenously into Rag2^{-/-}Il2rg^{-/-} mice. Two weeks later, splenocytes, bone marrow (BM) and peripheral blood cells (PB) were analyzed by flow cytometry fro ITNKs. (B) Most ITNKs in the spleen were CD8⁺. Numbers in gates refer to percentages. Data are representative of three experiments. (C) ITNKs had complete Bcl11b deletion whereas donor derived NKp46 cells still retained at least one copy of the foxed allele. PCR data are representative of two individual experiments. (D) ITNKs were also found in bone marrow and peripheral blood. About 1.0% of bone marrow and 6-7% of peripheral white blood cells expressed NKp46 and thus ITNKs in the recipients injected with Bcl11b-deficient DP thymocytes. (E) Expression of additional NK cell surface markers on the in vivo reprogrammed ITNKs. The in vivo iTNKs expressed more NK-specific receptors such as Ly49C/I and Ly49G2. (F) ITNKs prevented tumour metastasis. Rag2^{-/-}Il2rg^{-/-} recipients were transplanted with treated (+OHT) or untreated (−OHT) flox/flox DP thymocytes or PBS. Recipients were subsequently injected intravenously with 5×10⁴ B16F10 melanoma cells. Lung tumour colonies were enumerated two weeks after tumour challenge. Experiment was performed twice. (G) Plot shows inverse correlation between the percentage of ITNK cells (squares) obtained from recipient mice following in vivo reprogramming and tumor challenge and the number of lung colonies (circles) observed. Data are individual mice and are representative of two independent experiments, each with 5 mice per group. Chart shows that in vivo the percentages of ITNKs in spleen (squares) correlated with reduction of metastatic sites (+OHT circles) in the Rag2^{-/-}Il2γc^{-/-} mice after injection of OHT treated DP thymocytes. The −OHT squares and circles represent iTNKs and the metastatic sites respectively in recipient mice that were injected OHT untreated DP thymocytes. In mice injected with OHT-treated DP thymocytes, about 4% of splenocytes were iTNKs. FIG. 12. A working model showing that Bcl11b acts downstream of Notch signaling and promotes T cell development and maintains T cell identity.

FIG. 13. Bcl11b is expressed in early T cell precursors and is essential for T cell differentiation.
a. Expression of Bcl11b in thymocytes from Bcl11b-lacZ knock-in mice using the fluorescent substrate FDG. Almost all of the DN2-DN4 thymocytes were stained positively for FDG. However a significant DN1 population did not express Bcl11b.
b. Detection of Bcl11b expression in the five DN1 subpopulations. Approximately half of the DN1a and DN1b thymocytes, which were CD117⁺ and were thought to contain the true T cell progenitors, expressed Bcl11b.
c. Top, acute loss of Bcl11b caused DN1 thymocytes to express NK-specific genes NK1.1 and NKp46 on OP9-DL1 stromal cells. Bottom, deleting Bcl11b in DN2 thymocytes gave rise to the same phenotype of losing T cell differentiation potential and converting to NK-like cells.

FIG. 14.
a. Left panel: different double negative (DN) thymocyte populations defined by expression of CD25 and CD44. Right panel: five subpopulations of DN1 thymcoytes based on expression of CD24 and CD117 (c-Kit).
b. Flow chart of analyzing Bcl11b-deficient DN1 thymocytes. The Bcl11b-deficient cells (+OHT) acquired NK properties while the untreated ones (−OHT) proliferated and differentiated into T cells on OP9-DL1 stromal cells.

FIG. 15.
a. Double positive (DP) thymocytes expressed NKp46 after Bcl11b deletion. The untreated DP cells died in T cell media about one week after plated on OP9-DL1 stromal cells (not shown).
b. Purified CD8 single positive cells (−OHT) proliferated on OP9-DL1 stromal cells. They did not express NKp46. Once Bcl11b was deleted, 38% of the cells now expressed NKp46 which killed the stromal cells. Note that these iTNKs still expressed Tcrβ and CD8.
c. Purified CD4 single positive cells (−OHT) growing in T cell media (left). Bcl11b deletion (+OHT) caused these CD4 T cells to express NKp46. Note that most of the cells now did not express CD4 anymore.

### Materials and Methods

1.1.1 Mice

The Bcl11b conditional knockout targeting vector was constructed using recombineering (Liu et al., 2003), and the mice (Bcl11b^{flox/flox}) were made according to a standard gene targeting approach in ES cells. The Bcl11b^{flox/flox} mice were crossed to Cre-ERT2 mice to generate Cre-ERT2; Bcl11b^{flox/flox} mice. Cre-ERT2; mice were a mixed C57BL/6J and 129S5 genetic background. A SA-lacZ cassette was targeted into the intron 3 of Bcl11b gene in Bcl11b-lacZ reporter mice (Song-Choon Lee and Pentao Liu, unpublished). All mice were NK1.1⁺ by flow cytometry, suggesting that they had inherited the C57BL/6 haplotype at the NK gene complex. Bcl11b tdTomato reporter mice were constructed by inserting the tdTomato cassette into the 3′ UTR of Bcl11b. Bcl11b tdTomato mice are on a C57BL/6 background. Rag2^{-/-}Il2rg^{-/-} are on a C57BL/6 background. Both C57BL/6 and 129S5 have the H-2^{b} haplotype at the MHC. All animal experiments were performed in accordance with the UK 1986 Animals Scientific Procedure Act and local institute ethics committee regulations.

1.1.2 Reprogramming of T Cells to ITNKS In Vivo Flox/Flox

To test for the in vivo reprogramming of endogenous T cells to ITNK, Cre-ERT2; Bcl11b^{flox/flox} and Cre-ERT2; Bcl11b^{flox/+} mice were given 3 doses of 1 mg Tamoxifen (indicated in the text as OHT) (Sigma) dissolved in sunflower oil by oral gavage on 3 consecutive days. Mice were analysed 2-3 weeks later. For the in vivo reprogramming of in vitro-treated thymocytes, thymocytes from Cre-ERT2; were treated with 4-hydroxytamoxifen (indicated in the text as OHT) (Sigma) or left untreated for 48 hours. 2−4×10⁶ DP thymocytes were then sorted and injected intravenously into Rag2^{-/-}Il2rg^{-/-} recipient mice without irradiation. At various time points thereafter, blood, bone marrow and/or splenocytes were prepared for analysis.

1.1.3 PCR Genotyping and qRT-PCR

To extract genomic DNA, sorted cells were incubated in 400 μl of lysis buffer (50 mM Tris with pH 8.0, 100 mM NaCl, 25 mM EDTA with pH 8.0, 0.5% SDS, and 0.5 mg/ml Proteinase K) at 65° C. for 2 hrs. Genomic DNA was precipitated by adding 500 μl of isopropanol into cell lysis buffer. After centrifugation, DNA was washed once with 500 μl 70% ethanol and air dried before being re-suspended as template for PCR. The Bcl11b cko allele and the deletion after Cre-loxP recombination were detected by PCR with primers described in Table 4. PCR primers to detect TCRβ D-J and V-DJ are also listed in Table 4. For qRT-PCR, RNA was isolated using the RNAqueous Micro Kit (Ambion) from FACS sorted cells. After DNase I treatment, RNA was reverse transcribed to make cDNA with Super Script 11 (Invitrogen). qRT-PCR was performed with either SYBR (Invitrogen) or Taqman Master Mix (ABgene). cDNA input was standardized and PCR was performed for 40 cycles. Primers for qRT-PCR are listed in Table 4.

FDG Staining

For FDG staining, cells were first surface stained as above. Cells were then warmed at for 5 minutes before 20 μl pre-warmed FDG (Sigma) was added for a further 1 minute. The reaction was quenched by addition of 2.0 ml ice-cold PBS plus 1% BSA, and the cells were incubated on ice for a further 30 minutes. The cells were centrifuged and resuspended in PBS before analysis.

1.1.4 Flow Cytometry and Cell Sorting

Cells from spleen, thymus and bone marrow were mechanically disrupted and the red blood cells were removed with ACK lysis buffer (Lonza). Blood was collected into EDTA tubes (Sarstedt). In vitro-cultured cells were collected and washed with PBS/1% BSA prior to antibody labelling. For all cells, Fc receptors were blocked with anti-CD16 (2.4G2) prior to antibody labelling. Antibodies to the following antigens were used: CD3s (145-2C11), CD4 (L3T4), CD8a (53-6.7), CD25 (PC61), CD44 (IM7), CD122 (TM-131), CD27 (LG.3A10), CD11b (M1/70), CD45.2 (104), TCRβ (H57-597), CD117 (2B8), NK1.1 (PK136), CD49b (DX5), NKp46 (29A1.4), Ly49C/I (5E6), Ly49G2 (4D11), Ly49D (4E5). All antibodies were from BD Biosciences or eBioscience. Cells were incubated with antibody for 30 minutes at 4° C. before being washed. In some cases biotinylated antibodies were revealed by incubation with fluorochrome-conjugated streptavidin for a further 20 minutes at 4° C. CD1d-restricted NKT were detected by labelling cells with CD1d-mouse IgGl Fc fusion protein (BD Biosciences) loaded with α-galactosylceramide (Kirin), followed by fluorochrome-conjugated anti-mouse IgGl (BD Biosciences). Data acquisition was performed using a FACSCalibur (BD Biosciences), LSR II (BD Biosciences) or a FC 500 (Beckman Coulter) with dead cells excluded based on scatter profile or DAPI inclusion. Analysis was performed using FlowJo (Tree Star) software. Sorting was performed using a MoFlo (DAKO) or FACS Aria (BD Biosciences).

1.1.5 OP9 Stromal Cell Culture

OP9 stromal cells were cultured in alpha-MEM (Sigma) with 10% FCS (heat inactivated at 56° C. for 30 min), 1% penicillin/streptomycin, and 2 mM L-glutamine (Life Technologies). OP9-DL1 stromal cells were cultured in alpha-MEM (Sigma) with 20% FCS, 1% penicillin/streptomycin, and 2 mM L-glutamine (Life Technologies). Cells were passaged every 2 to 3 days by trypsinization (Invitrogen). A monolayer (70%-80% confluent) of OP9 or OP9-DL1 cells was prepared 24 hours prior to co-culture.

1.1.6 OHT Treatment In Vitro

Thymocytes or splenocytes from Cre-ERT2; Bcl11b^{flox/flox} mice were cultured in T cell medium with 1 μM 4-hydroxytamoxifen (indicated in the text as OHT) at 37° C. for 48 hrs. After this time, cells were washed and resuspended with fresh media. T cell media: RPMI-1640, 10% FCS, 1% penicillin/streptomycin, 2 mM L-glutamine, 5 ng/ml muFlt-3L, 5 ng/ml hulL-7. All cytokines used in this study were purchased from PeproTech.

1.1.7 Reprogramming of T Cells to ITNKS In Vitro

After OHT treatment, thymocytes were sorted by FACS and co-cultured with OP9-DL1 in T cell culture media (3,000 cells per well in 24-well plates). To promote ITNK proliferation, 20 ng/ml mulL-15 or 100 ng/ml hulL-2 was supplemented in T cell medium as indicated. Every three days, half of the media was replaced with fresh T cell media with IL-15 or IL-2 as indicated in text. Every seven days, cells were collected by vigorous pipetting, filtered through cell strainers and transferred to new tissue culture plates pre-seeded with fresh OP9-DL1 stromal cells. Fourteen days after OHT treatment, cells were collected and analyzed by FACS. For analysis of ITNK production in myeloid cell differentiation conditions, IMDM was used supplemented with 10% FCS, 1% penicillin/streptomycin, 2 mM L-gluatamine, 1 ng/ml hulL-7, 5 ng/ml muFlt-3L, 10 ng/ml hulL-3, hulL-6, stem cell factor (muSCF), and granulocyte/macrophage colony-stimulating factor (muGM-CSF). Cells were cultured on OP9 stromal cells. For analysis of ITNK production in B cell differentiation conditions, IMDM was used supplemented with 10% FCS, 1% penicillin/streptomycin, 2 mM L-gluatamine, 5 ng/ml hulL-7, 5 ng/ml muFlt-3L. Cells were cultured on OP9 stromal cells.

1.1.8 Reprogramming of Single Thymocyte to ITNKs

Thymocytes of Cre-ERT2; Bcl11b^{flox/flox} were treated with OHT as above. Single DN3 thymocytes were sorted directly into individual wells of a 96-well plate pre-seeded with OP9-DL1 stromal cells in T cell medium supplemented with 100 ng/ml hulL-2. Medium was changed every three days. After 10-14 days cells were analyzed in flow cytometry. Genomic DNA was extracted for genotyping of the Bcl11b locus and for amplifying 6TCR rearrangement with PCR.

1.1.9 Tumour Cell Killing Assay

B16F10 melanoma (H-2^{b}), RMA lymphoma and RMA-S lymphoma (N-2^{b} TAP-1-deficient variant) were maintained in RPMI-1640, 5% FCS, 1% penicillin/streptomycin, 2 mM L-glutamine. For killing assays, target cells were washed and incubated with 0.1 μCi Na₂⁵¹CrO₄ (Perkin Elmer) for 45 mins. at 37° C. The cells were then washed and added in triplicate to effector cells at the indicated E:T ratio. Plates were incubated for 4 hours at 37° C. before the supernatant was tested for chromium release in a scintillation counter. Percent specific lysis was calculated as (experimental release—spontaneous release)/(maximum release—spontaneous release)×100.

T Cells to iTNKs In Vivo

Thymocytes from Cre-ERT2; b^{flox/flox} were treated with OHT as above. 2−4×10⁶ DP thymocytes were sorted and injected intravenously into Rag2^{-/-}Il2γc^{-/-} recipient mice without irradiation. At various time points thereafter, blood and/or splenocytes were prepared for analysis.

1.1.10 ITNK Ex Vivo Expansion and LAK Culturing

For ex vivo expansion, splenic ITNK cells were enriched using the NK Isolation Kit (Miltenyi) and cultured for 6-9 days at 1×10⁶ cells/ml in RPMI 1640 medium containing 10% FCS/50 μM 2-mercaptoethanol/2.0 mM L-glutamine and 1000 U/ml hIL-2 (Chiron). The cells were split every two days and supplemented with fresh IL-2. Purity was always >90%. For culturing reprogrammed ITNK cells ex vivo, whole splenocytes were cultured without pre-enrichment.

1.1.11 Tumour Experiments In Vivo

After OHT treatment, 2−4×10⁶ DP T cells were sorted from Cre-ERT2; Bcl11b^{flox/flox} thymocytes and injected intravenously into each Rag2^{-/-}Il2rg^{-/-} recipient mouse without irradiation. Two weeks later, 5×10⁴ B16F10 melanoma cells were injected intravenously and the lung colonies were enumerated 14 days after tumour inoculation.

### Calcium Flux Experiments

flox/flox or flox/+ mice were treated with Tamoxifen to derive in vivo-reprogrammed ITNK as described above and splenocytes were analyzed 4-5 weeks later. Splenocytes were either stained directly with antibodies to NKp46, NK1.1, CD8 and CD3 for phenotyping, or loaded with 2 μM Indo-1 (Invitrogen), washed and stained with antibodies to NKp46, NK1.1 and CD8. Data was then acquired using an LSR II flow cytometer gating on lymphocytes, measuring Indo-1 (violet)/Indo-1 (blue) ratio against time. Unstimulated cells were run to establish the baseline Indo-1 (violet)/Indo-1 (blue) fluorescence before acquisition was interrupted, anti-CD3 (145-2C11; μg/ml) added and acquisition continued. Acquisition was interrupted again and cross-linking anti-hamster IgG secondary antibody was added before continuing. Ionomycin (1 μg/ml) was added at the end of the acquisiton to serve as a positive control.

1.1.12 Gene Expression Analysis

RNA was extracted using the RNAqueous Micro Kit (Ambion) from FACS sorted cells. Quality and quantity of RNA samples was tested with Bioanalyzer. Total RNA was amplified using the Illumina Total Prep RNA Amplification Kit (Ambion) according to the manufacture's instructions. The biotinlated cRNA (1500 ng per sample) was applied to Illumina Mouse-6 Expression BeadChips and hybridized overnight at 58° C. Chips were washed, detected and scanned according to the manufacture's instruction and the scanner output imported into BeadStudio software (Illumina).

### Chromatin Immunoprecipitation

Chromatin immunoprecipitation was performed as previously described (38). Control IgG and the CSL antibody were purchased from Abcam. Genomic DNA was purified with Qiaquick PCR purification kit (QIAGEN) and specific genomic DNA regions were quantified by real-time quantitative PCR with Taqman (ABI) or SYBR Green (Invitrogen). Input DNA was used as a standard curve to quantify concentration of DNA recovered after IP. The amount of DNA recovered from each ChIP sample was presented as relative to the control IgG. Primers used in this assay are listed in table 4.

TABLE 1
ITNK vs. DN3

| | p-value | Ratio | Fold-Change |
|---|---|---|---|
| Column ID | (ITNK vs. DN3) | (ITNK vs. DN3) | (ITNK vs. DN3) |
| FCER1G | 1.61E−08 | 38.85426542 | 38.8542 |
| ROG | 3.56E−09 | 38.51902069 | 38.519 |
| UPP1 | 1.59E−11 | 27.95435613 | 27.9543 |
| IFITM1 | 3.53E−06 | 27.42649015 | 27.4265 |
| XCL1 | 1.21E−06 | 25.36912071 | 25.3691 |
| SERPINA3G | 7.75E−08 | 21.14875825 | 21.1487 |
| SCIN | 1.90E−08 | 20.78544021 | 20.7854 |
| NKG7 | 1.76E−08 | 20.18204202 | 20.182 |
| AQP9 | 1.71E−07 | 18.25220532 | 18.2522 |
| KLRD1 | 7.30E−09 | 17.17811645 | 17.1781 |
| LGALS3 | 7.17E−09 | 15.91702772 | 15.917 |
| AVIL | 9.13E−07 | 13.61854192 | 13.6185 |
| IFITM3 | 1.54E−07 | 13.57143051 | 13.5714 |
| TYROBP | 1.57E−09 | 13.52446102 | 13.5245 |
| GADD45G | 4.52E−08 | 13.52446102 | 13.5245 |
| CD160 | 3.07E−07 | 12.64065893 | 12.6407 |
| IFITM2 | 4.83E−06 | 11.27456728 | 11.2746 |
| CTSW | 3.28E−06 | 9.798061943 | 9.79809 |
| 9130404D14RIK | 6.12E−09 | 9.679979866 | 9.67995 |
| LOC270152 | 2.41E−07 | 9.530162966 | 9.53016 |
| BC025206 | 1.38E−08 | 9.063060777 | 9.06307 |
| VIM | 3.49E−08 | 8.891971439 | 8.89195 |
| NFIL3 | 2.83E−06 | 8.426871608 | 8.42689 |
| AMICA1 | 9.38E−08 | 8.267810932 | 8.26778 |
| LTA | 1.13E−11 | 8.210652501 | 8.21067 |
| GLRX1 | 2.07E−06 | 8.027743883 | 8.02777 |
| LITAF | 1.96E−07 | 7.727497527 | 7.72749 |
| CCR5 | 6.07E−09 | 7.323324789 | 7.32333 |
| LMNA | 5.83E−08 | 7.235052382 | 7.23503 |
| BC049975 | 2.32E−08 | 6.988559728 | 6.98858 |
| P2RY14 | 1.44E−07 | 6.797495803 | 6.79748 |
| WBSCR5 | 8.76E−08 | 6.486766995 | 6.48677 |
| LAG3 | 8.41E−08 | 6.190263953 | 6.19026 |
| LY6A | 1.40E−05 | 5.989781433 | 5.98977 |
| E030006K04RIK | 6.54E−09 | 5.948379959 | 5.94839 |
| 9130211I03RIK | 4.39E−06 | 5.87668367 | 5.87667 |
| 1300002F13RIK | 7.18E−06 | 5.866513355 | 5.8665 |
| LOC381140 | 2.90E−07 | 5.84620961 | 5.8462 |
| GPR114 | 2.07E−05 | 5.78573123 | 5.78573 |
| 2310067E08RIK | 3.25E−07 | 5.725901114 | 5.72589 |
| CDKN2B | 6.18E−06 | 5.686341408 | 5.68634 |
| IDB2 | 1.14E−08 | 5.637296353 | 5.63728 |
| GOLPH2 | 4.35E−09 | 5.608053164 | 5.60805 |
| PLCG2 | 2.95E−09 | 5.550036353 | 5.55005 |
| 1500031H04RIK | 1.83E−07 | 5.492634377 | 5.49264 |
| 1110018K11RIK | 7.30E−10 | 5.388947269 | 5.38893 |
| CD9 | 9.16E−09 | 5.388947269 | 5.38893 |
| LOC381319 | 6.47E−06 | 5.323963158 | 5.32396 |
| SYTL2 | 3.83E−10 | 5.250666835 | 5.25066 |
| SLC2A6 | 1.27E−07 | 5.223432317 | 5.22344 |
| OSBPL3 | 3.09E−11 | 5.187367722 | 5.18736 |
| 2210411K11RIK | 2.39E−06 | 5.080603779 | 5.0806 |
| LRRK1 | 1.15E−07 | 5.045510505 | 5.04551 |
| S100A6 | 3.80E−07 | 4.967438441 | 4.96743 |
| KLRE1 | 8.01E−08 | 4.933107068 | 4.93312 |
| PGLYRP1 | 6.39E−06 | 4.933107068 | 4.93312 |
| GLRX | 6.59E−06 | 4.873650608 | 4.87364 |
| MYO1F | 8.46E−09 | 4.839966507 | 4.83998 |
| LOC269941 | 9.61E−10 | 4.831594764 | 4.8316 |
| TRAF1 | 1.82E−06 | 4.69946896 | 4.69948 |
| EMILIN2 | 0.000282473 | 4.691333699 | 4.69134 |
| TNFRSF9 | 1.19E−07 | 4.67510367 | 4.67511 |
| CD52 | 9.20E−05 | 4.618745641 | 4.61874 |
| PLSCR1 | 2.75E−09 | 4.602758894 | 4.60276 |
| BHLHB2 | 5.80E−06 | 4.570968863 | 4.57097 |
| S100A1 | 8.07E−08 | 4.55514458 | 4.55515 |
| LGALS1 | 4.96E−07 | 4.523699663 | 4.52369 |
| 2310046K01RIK | 3.01E−06 | 4.4691539 | 4.46915 |
| CAPG | 5.03E−09 | 4.453688322 | 4.45369 |
| C330008K14RIK | 4.31E−07 | 4.430601277 | 4.43059 |
| TNFRSF11B | 0.000351472 | 4.384772562 | 4.38477 |
| CCL4 | 8.44E−05 | 4.362031136 | 4.36203 |
| SIAT10 | 6.50E−06 | 4.339411402 | 4.33941 |
| HBA-A1 | 1.28E−06 | 4.301981923 | 4.30198 |
| ROM1 | 1.73E−08 | 4.272262761 | 4.27226 |
| 1190002C06RIK | 2.57E−07 | 4.198875541 | 4.19887 |
| F2R | 2.39E−05 | 4.184345527 | 4.18434 |
| RGS1 | 5.76E−05 | 4.184345527 | 4.18434 |
| CD69 | 2.26E−05 | 4.177091992 | 4.17709 |
| CISH | 1.63E−06 | 4.155429692 | 4.15544 |
| DAPK2 | 9.71E−09 | 4.133888377 | 4.13389 |
| SH3BP2 | 3.58E−08 | 4.098226288 | 4.09823 |
| GCNT1 | 2.49E−08 | 4.069921247 | 4.06992 |
| HAVCR2 | 5.18E−05 | 4.062877086 | 4.06287 |
| DUSP6 | 3.27E−09 | 4.041808467 | 4.04181 |
| CTNNA1 | 4.08E−08 | 3.993068034 | 3.99307 |
| BC024955 | 6.91E−10 | 3.917681673 | 3.91768 |
| ITGB7 | 0.000152314 | 3.917681673 | 3.91768 |
| MLKL | 7.92E−08 | 3.877156183 | 3.87716 |
| SERPINE2 | 2.49E−05 | 3.870448353 | 3.87045 |
| LY6G5B | 7.00E−06 | 3.863748764 | 3.86375 |
| PPP3CC | 5.92E−09 | 3.850374449 | 3.85038 |
| LOC218482 | 1.61E−08 | 3.837062959 | 3.83706 |
| A430006M23RIK | 3.38E−06 | 3.7776762 | 3.77768 |
| 2410008K03RIK | 3.43E−08 | 3.732137059 | 3.73213 |
| FURIN | 1.27E−06 | 3.732137059 | 3.73213 |
| F2RL2 | 9.14E−05 | 3.732137059 | 3.73213 |
| GPR18 | 2.97E−06 | 3.712779387 | 3.71278 |
| HGFAC | 7.05E−05 | 3.70635306 | 3.70635 |
| S100A10 | 4.21E−06 | 3.693525988 | 3.69353 |
| APOB48R | 4.85E−06 | 3.68074675 | 3.68075 |
| OSM | 1.62E−05 | 3.68074675 | 3.68075 |
| AIM1L | 2.96E−06 | 3.674376734 | 3.67438 |
| IL18R1 | 3.56E−07 | 3.661662395 | 3.66167 |
| NT5E | 2.59E−08 | 3.655331484 | 3.65533 |
| IFNG | 3.30E−06 | 3.63637686 | 3.63637 |
| H2-Q8 | 0.000127421 | 3.630080297 | 3.63008 |
| FXYD4 | 1.04E−07 | 3.617513104 | 3.61752 |
| PILRB | 3.51E−05 | 3.592534713 | 3.59253 |
| PLP2 | 3.87E−08 | 3.580097522 | 3.5801 |
| MT1 | 0.000577989 | 3.580097522 | 3.5801 |
| DOK2 | 7.55E−08 | 3.567707962 | 3.56771 |
| 0610037M15RIK | 7.75E−06 | 3.549220591 | 3.54922 |
| 2310047C17RIK | 4.43E−05 | 3.549220591 | 3.54922 |
| S100A11 | 2.00E−07 | 3.530811628 | 3.53081 |
| FES | 1.98E−08 | 3.518599316 | 3.5186 |
| BC029169 | 3.49E−07 | 3.500346534 | 3.50035 |
| TNF | 5.66E−10 | 3.482197266 | 3.4822 |
| LRP12 | 7.21E−05 | 3.482197266 | 3.4822 |
| IER3 | 0.000215344 | 3.476169123 | 3.47617 |
| NAPSA | 2.95E−05 | 3.470149772 | 3.47015 |
| ANXA2 | 3.41E−06 | 3.434266424 | 3.43426 |
| PRSS19 | 1.77E−05 | 3.428320671 | 3.42832 |
| OSTF1 | 5.84E−09 | 3.381119827 | 3.38112 |
| GVIN1 | 0.000422456 | 3.36941982 | 3.36942 |
| 1110007C02RIK | 2.44E−08 | 3.340348064 | 3.34035 |
| MAPKAPK3 | 1.77E−07 | 3.311532412 | 3.31153 |
| CD244 | 7.87E−08 | 3.277280906 | 3.27728 |
| F630022B06RIK | 4.89E−06 | 3.271608977 | 3.27161 |
| ID2 | 1.03E−07 | 3.265945981 | 3.26594 |
| GPR68 | 6.05E−06 | 3.265945981 | 3.26594 |
| GLIPR1 | 4.48E−07 | 3.260291927 | 3.26029 |
| PDGFA | 1.76E−06 | 3.254646822 | 3.25464 |
| PKP3 | 1.71E−08 | 3.254646822 | 3.25464 |
| D10BWG1379E | 5.49E−08 | 3.254646822 | 3.25464 |
| SLC39A4 | 4.73E−08 | 3.215403067 | 3.2154 |
| TES | 1.92E−07 | 3.182149415 | 3.18215 |
| EGR1 | 0.0012963 | 3.182149415 | 3.18215 |
| 1110004P15RIK | 5.91E−05 | 3.176640258 | 3.17664 |
| B4GALNT4 | 4.86E−07 | 3.149229384 | 3.14923 |
| CDKN1A | 0.000553492 | 3.149229384 | 3.14923 |
| D2ERTD217E | 7.91E−08 | 3.079083172 | 3.07908 |
| NCF4 | 5.37E−06 | 3.073754745 | 3.07375 |
| LOC381924 | 1.33E−06 | 3.047229002 | 3.04723 |
| 170002G04RIK | 2.02E−06 | 3.041954638 | 3.04196 |
| AA467197 | 0.00100238 | 3.03668928 | 3.03669 |
| SGK | 5.37E−08 | 3.020947248 | 3.02095 |
| BC021614 | 5.58E−05 | 3.015717922 | 3.01571 |
| LOC385953 | 1.27E−10 | 3.005277267 | 3.00528 |
| CDKN2A | 8.87E−06 | 2.989697502 | 2.9897 |
| 2610009E16RIK | 2.28E−05 | 2.953651304 | 2.95365 |
| HIST1H1C | 0.00103646 | 2.928171942 | 2.92817 |
| DCI | 1.02E−08 | 2.912989018 | 2.91299 |
| NFKB1 | 1.21E−07 | 2.902909296 | 2.90291 |
| TPST2 | 2.16E−06 | 2.902909296 | 2.90291 |
| TRF | 2.76E−06 | 2.89788715 | 2.89788 |
| HK2 | 3.64E−06 | 2.887861452 | 2.88786 |
| PDZK1 | 5.44E−10 | 2.88285795 | 2.88286 |
| GNG2 | 6.86E−09 | 2.872886274 | 2.87288 |
| S100A4 | 8.62E−08 | 2.848102167 | 2.8481 |
| ZFP608 | 8.21E−08 | 2.838248233 | 2.83825 |
| 2210008N01RIK | 6.64E−08 | 2.83333475 | 2.83333 |
| SH3BGRL3 | 4.55E−05 | 2.828430249 | 2.82843 |
| MYO1G | 2.01E−06 | 2.823526754 | 2.82353 |
| 1110019C08RIK | 1.34E−05 | 2.818640232 | 2.81864 |
| S100A13 | 8.29E−05 | 2.818640232 | 2.81864 |
| PPAP2C | 3.95E−06 | 2.813762676 | 2.81376 |
| MYO1E | 7.25E−08 | 2.808886192 | 2.80889 |
| IFI30 | 1.07E−06 | 2.799168087 | 2.79917 |
| LTB4R1 | 0.000868775 | 2.784654327 | 2.78466 |
| TMEM126A | 3.48E−08 | 2.775025877 | 2.77502 |
| 1110020C13RIK | 7.24E−08 | 2.775025877 | 2.77502 |
| CD7 | 7.40E−06 | 2.775025877 | 2.77502 |
| 4933439K08RIK | 1.06E−08 | 2.751084615 | 2.75108 |
| E030003N15RIK | 2.16E−05 | 2.732083678 | 2.73208 |
| CCL5 | 0.0117888 | 2.732083678 | 2.73208 |
| 7420404O03RIK | 1.10E−05 | 2.722629407 | 2.72263 |
| 4930486L24RIK | 1.95E−06 | 2.717915685 | 2.71791 |
| BC022224 | 9.36E−07 | 2.713210895 | 2.71321 |
| 5330403J18RIK | 5.98E−06 | 2.708507694 | 2.70851 |
| FXYD5 | 4.01E−06 | 2.703820769 | 2.70382 |
| A430038C16RIK | 2.01E−06 | 2.699142752 | 2.69914 |
| GPC1 | 0.00472373 | 2.675857345 | 2.67586 |
| AHNAK | 7.45E−07 | 2.666595557 | 2.6666 |
| EMP1 | 0.000268268 | 2.666595557 | 2.6666 |
| CX3CR1 | 3.25E−09 | 2.661981579 | 2.66198 |
| 2810032E02RIK | 4.29E−07 | 2.661981579 | 2.66198 |
| LOC327957 | 0.000272484 | 2.657369417 | 2.65737 |
| FCGR3 | 3.00E−05 | 2.625333417 | 2.62533 |
| CLNK | 1.01E−08 | 2.620785449 | 2.62079 |
| HVCN1 | 0.000222656 | 2.620785449 | 2.62079 |
| BSCL2 | 5.79E−06 | 2.616246367 | 2.61625 |
| LGALS3BP | 6.49E−06 | 2.616246367 | 2.61625 |
| CYP51 | 7.38E−07 | 2.607194815 | 2.6072 |
| PIM3 | 6.98E−05 | 2.593677134 | 2.59368 |
| BC03881 | 7.63E−09 | 2.584707321 | 2.58471 |
| LOC383981 | 0.00132323 | 2.584707321 | 2.58471 |
| PDLIM7 | 5.33E−07 | 2.580232324 | 2.58023 |
| SLC24A3 | 6.09E−05 | 2.580232324 | 2.58023 |
| HHEX | 5.95E−07 | 2.575766162 | 2.57576 |
| D930046M13RIK | 1.74E−05 | 2.575766162 | 2.57576 |
| BC087945 | 7.68E−07 | 2.57130221 | 2.5713 |
| 9830144J08RIK | 1.24E−07 | 2.566853705 | 2.56685 |
| DP1 | 1.29E−06 | 2.566853705 | 2.56685 |
| E130012A19RIK | 1.31E−05 | 2.562407433 | 2.56241 |
| 4631423F02RIK | 7.66E−05 | 2.540301889 | 2.5403 |
| 4930555L03RIK | 4.40E−08 | 2.535902033 | 2.5359 |
| FOSL2 | 6.92E−06 | 2.535902033 | 2.5359 |
| ZFP296 | 2.68E−09 | 2.518384205 | 2.51839 |
| F730045P10RIK | 0.000293728 | 2.518384205 | 2.51839 |
| PEA15 | 5.59E−08 | 2.509674796 | 2.50967 |
| ITGAE | 0.000534865 | 2.50533009 | 2.50533 |
| A530050E01RIK | 1.01E−05 | 2.50533009 | 2.50533 |
| SCL0001419.1_32 | 1.67E−07 | 2.500994145 | 2.50099 |
| SPP1 | 0.00129868 | 2.496660716 | 2.49666 |
| ASB2 | 2.54E−05 | 2.492336067 | 2.49234 |
| CCNG1 | 4.25E−05 | 2.492336067 | 2.49234 |
| TUBA6 | 1.47E−06 | 2.483713052 | 2.48372 |
| SDF2L1 | 7.56E−05 | 2.483713052 | 2.48372 |
| RHOF | 9.16E−06 | 2.47941466 | 2.47942 |
| 1110030J09RIK | 2.72E−10 | 2.45802311 | 2.45803 |
| EGR3 | 0.00128127 | 2.45802311 | 2.45803 |
| CXCR3 | 5.82E−05 | 2.453770955 | 2.45377 |
| ALDOA | 0.000483723 | 2.449521486 | 2.44952 |
| GCNT2 | 3.22E−08 | 2.44528073 | 2.44528 |
| MVP | 4.37E−07 | 2.44528073 | 2.44528 |
| C130027E04RIK | 5.53E−07 | 2.44528073 | 2.44528 |
| SEC61B | 1.47E−09 | 2.436819366 | 2.43682 |
| E430036I04RIK | 2.87E−07 | 2.43259877 | 2.4326 |
| AI481100 | 9.30E−05 | 2.424183656 | 2.42419 |
| CD63 | 0.000436295 | 2.41998911 | 2.41999 |
| DEGS | 4.85E−07 | 2.415797382 | 2.4158 |
| LOC385699 | 1.78E−05 | 2.415797382 | 2.4158 |
| EG331493 | 3.45E−06 | 2.415797382 | 2.4158 |
| A930008A22RIK | 6.04E−07 | 2.411614335 | 2.41162 |
| 4930504E06RIK | 0.000436715 | 2.411614335 | 2.41162 |
| LOC212399 | 1.73E−06 | 2.407439952 | 2.40744 |
| AI850995 | 3.62E−06 | 2.407439952 | 2.40744 |
| CTGF | 0.000743838 | 2.407439952 | 2.40744 |
| KIRL2 | 3.13E−06 | 2.394957178 | 2.39496 |
| 1700017I11RIK | 6.32E−08 | 2.390811633 | 2.39081 |
| 2310037P21RIK | 2.51E−06 | 2.386669022 | 2.38667 |
| UAP1L1 | 3.29E−06 | 2.382540741 | 2.38254 |
| D14ERTD449E | 0.000867516 | 2.382540741 | 2.38254 |
| BC023892 | 4.42E−09 | 2.378415405 | 2.37841 |
| AA175286 | 0.000409847 | 2.361983405 | 2.36199 |
| PPIB | 5.05E−09 | 2.357895531 | 2.3579 |
| GPR34 | 1.68E−07 | 2.357895531 | 2.3579 |
| IRAK2 | 7.34E−05 | 2.357895531 | 2.3579 |
| SH2D1B1 | 0.000605208 | 2.353810702 | 2.35381 |
| HSD11B1 | 6.66E−05 | 2.353810702 | 2.35381 |
| LOC328703 | 3.58E−06 | 2.349740001 | 2.34974 |
| BC004728 | 5.49E−07 | 2.349740001 | 2.34974 |
| LOC215405 | 4.47E−05 | 2.349740001 | 2.34974 |
| RAB3D | 1.16E−05 | 2.337551835 | 2.33755 |
| SULT2B1 | 0.000164735 | 2.337551835 | 2.33755 |
| EVI2A | 2.55E−07 | 2.333509902 | 2.33351 |
| TSPO | 2.22E−06 | 2.333509902 | 2.33351 |
| TNFRSF18 | 2.31E−05 | 2.333509902 | 2.33351 |
| EG630499 | 0.000147665 | 2.329465644 | 2.32947 |
| SERPINB6A | 0.00133703 | 2.32543538 | 2.32543 |
| SYPL | 6.57E−07 | 2.321408259 | 2.32141 |
| 8030402P03RIK | 0.000483123 | 2.321408259 | 2.32141 |
| HAAO | 3.04E−05 | 2.317389692 | 2.31739 |
| NRGN | 5.54E−05 | 2.313374311 | 2.31338 |
| TAF9B | 1.33E−06 | 2.309372821 | 2.30937 |
| 9930117H01RIK | 6.56E−05 | 2.309372821 | 2.30937 |
| GBP2 | 0.000466109 | 2.309372821 | 2.30937 |
| AW212394 | 7.13E−06 | 2.30537452 | 2.30537 |
| KIT | 0.000138895 | 2.301379447 | 2.30138 |
| A630086H07RIK | 3.07E−05 | 2.297398197 | 2.2974 |
| ANK | 1.14E−07 | 2.293420178 | 2.29342 |
| BATF | 6.24E−06 | 2.293420178 | 2.29342 |
| TIAM1 | 1.13E−07 | 2.289450669 | 2.28945 |
| TCRD-V1 | 6.14E−06 | 2.285484431 | 2.28548 |
| ARL6IP5 | 2.09E−07 | 2.285484431 | 2.28548 |
| EHD4 | 3.43E−05 | 2.285484431 | 2.28548 |
| N4WBP5-PENDING | 9.39E−08 | 2.281526706 | 2.28153 |
| B3GNT8 | 1.44E−07 | 2.281526706 | 2.28153 |
| BLR1 | 8.56E−06 | 2.281526706 | 2.28153 |
| NDFIP1 | 2.11E−06 | 2.261844715 | 2.26184 |
| PRDX4 | 1.09E−05 | 2.261844715 | 2.26184 |
| SNAG1 | 4.20E−07 | 2.250118694 | 2.25012 |
| B4GALNT2 | 0.000956597 | 2.250118694 | 2.25012 |
| TRPM6 | 9.96E−08 | 2.238448487 | 2.23845 |
| CXCL9 | 0.00820156 | 2.230703854 | 2.23071 |
| 0610009O03RIK | 8.23E−09 | 2.222987918 | 2.22299 |
| PRR7 | 1.26E−06 | 2.207632226 | 2.20763 |
| A630077B13RIK | 3.14E−05 | 2.207632226 | 2.20763 |
| SLC19A2 | 4.63E−05 | 2.207632226 | 2.20763 |
| 2810440J20RIK | 7.67E−07 | 2.192381474 | 2.19238 |
| MED10 | 3.04E−06 | 2.192381474 | 2.19238 |
| COMT | 8.15E−09 | 2.188586086 | 2.18859 |
| PLTP | 6.19E−07 | 2.188586086 | 2.18859 |
| 2310010I15RIK | 1.21E−08 | 2.181015568 | 2.18102 |
| 0610039P13RIK | 0.000646447 | 2.181015568 | 2.18102 |
| VPS29 | 1.09E−07 | 2.177240435 | 2.17724 |
| AI847670 | 1.60E−06 | 2.16595082 | 2.16595 |
| ASAH1 | 3.80E−08 | 2.162199562 | 2.1622 |
| B830021E24RIK | 1.42E−05 | 2.162199562 | 2.1622 |
| SRGAP2 | 4.27E−06 | 2.158456617 | 2.15846 |
| IQGAP2 | 0.000141764 | 2.158456617 | 2.15846 |
| LASP1 | 2.73E−06 | 2.154717323 | 2.15472 |
| CORO1C | 7.53E−07 | 2.150990962 | 2.15099 |
| H2-Q6 | 3.02E−06 | 2.150990962 | 2.15099 |
| 9130604K18RIK | 4.57E−06 | 2.147263635 | 2.14726 |
| FNBP1 | 2.88E−08 | 2.143549203 | 2.14355 |
| TMPIT | 3.47E−06 | 2.139833863 | 2.13984 |
| H2-Q7 | 5.84E−05 | 2.136131381 | 2.13613 |
| 0610007H07RIK | 2.48E−06 | 2.132432594 | 2.13243 |
| CCND2 | 3.28E−06 | 2.125055251 | 2.12505 |
| SERTAD1 | 0.000141861 | 2.121376689 | 2.12138 |
| RAB19 | 7.99E−06 | 2.114035292 | 2.11404 |
| BAG3 | 0.00480518 | 2.110376934 | 2.11038 |
| VTI1B | 1.30E−05 | 2.10672234 | 2.10672 |
| CAPN2 | 2.23E−06 | 2.103075959 | 2.10307 |
| 2310057H16RIK | 6.11E−05 | 2.099433363 | 2.09943 |
| STX11 | 0.000109261 | 2.095798971 | 2.0958 |
| FTL1 | 0.00023523 | 2.095798971 | 2.0958 |
| ARF6 | 3.80E−07 | 2.092168386 | 2.09217 |
| 2900026A02RIK | 2.85E−07 | 2.088545996 | 2.08855 |
| CSTB | 3.81E−05 | 2.088545996 | 2.08855 |
| LOC383189 | 5.48E−07 | 2.084931781 | 2.08493 |
| CCL3 | 0.00511685 | 2.084931781 | 2.08493 |
| GLIPR2 | 6.01E−05 | 2.084931781 | 2.08493 |
| C330023F11RIK | 1.81E−06 | 2.081321389 | 2.08132 |
| SIRT3 | 2.69E−06 | 2.081321389 | 2.08132 |
| CAPNS1 | 3.44E−08 | 2.077719163 | 2.07772 |
| RBMS1 | 1.72E−07 | 2.077719163 | 2.07772 |
| 1110008P14RIK | 2.41E−07 | 2.077719163 | 2.07772 |
| MINA | 1.96E−08 | 2.07412078 | 2.07412 |
| CCDC132 | 2.24E−06 | 2.07412078 | 2.07412 |
| LOC234582 | 1.09E−06 | 2.066944188 | 2.06695 |
| KCTD10 | 9.73E−05 | 2.066944188 | 2.06695 |
| LOC240672 | 5.49E−06 | 2.056229656 | 2.05623 |
| A230057G18RIK | 9.28E−07 | 2.056229656 | 2.05623 |
| ELOVL1 | 3.81E−06 | 2.056229656 | 2.05623 |
| STX7 | 5.97E−06 | 2.052667339 | 2.05267 |
| BC017612 | 5.16E−06 | 2.052667339 | 2.05267 |
| ZBTB32 | 3.61E−05 | 2.052667339 | 2.05267 |
| H47 | 3.72E−06 | 2.049113144 | 2.04911 |
| TNFRSF22 | 7.52E−05 | 2.049113144 | 2.04911 |
| AI115600 | 1.65E−06 | 2.045567052 | 2.04557 |
| MYL6 | 1.28E−07 | 2.045567052 | 2.04557 |
| H2-GS17 | 0.000428176 | 2.042024872 | 2.04202 |
| CAPZB | 5.20E−07 | 2.038490783 | 2.03849 |
| SC4MOL | 3.38E−06 | 2.038490783 | 2.03849 |
| FHL2 | 6.33E−07 | 2.034960624 | 2.03496 |
| 3010031K01RIK | 4.37E−08 | 2.034960624 | 2.03496 |
| A330042I21RIK | 1.18E−06 | 2.034960624 | 2.03496 |
| D15MGI27 | 2.70E−05 | 2.034960624 | 2.03496 |
| RAB4A | 4.71E−08 | 2.024406242 | 2.02441 |
| DCXR | 0.000151877 | 2.024406242 | 2.02441 |
| AIM1 | 0.000114024 | 2.024406242 | 2.02441 |
| SEMA4A | 7.97E−05 | 2.017402111 | 2.0174 |
| XBP1 | 0.000154152 | 2.017402111 | 2.0174 |
| LOC383099 | 0.000417017 | 2.013912105 | 2.01391 |
| JUNB | 0.00325781 | 2.013912105 | 2.01391 |
| HRMT1L1 | 7.88E−06 | 2.01042607 | 2.01042 |
| GPR97 | 0.000165473 | 2.01042607 | 2.01042 |
| COTL1 | 0.000539041 | 2.01042607 | 2.01042 |
| 2310061N23RIK | 0.025362 | 2.006944026 | 2.00694 |
| 9130227C08RIK | 4.47E−06 | 2.00347001 | 2.00347 |
| AI840980 | 0.000521047 | 2.00347001 | 2.00347 |
| DYRK3 | 0.000241993 | 2.00347001 | 2.00347 |
| CASP1 | 0.00573828 | 2.00347001 | 2.00347 |
| TRBV11_AE000663_T_CELL_RECEPTOR_BETA_VARIABLE_11_{—} | 0.000105474 | 0.5 | −2 |
| C920004C08RIK | 0.0113298 | 0.5 | −2 |
| A930023F05RIK | 1.93E−06 | 0.499134003 | −2.00347 |
| PLEKHG2 | 1.52E−05 | 0.499134003 | −2.00347 |
| ABHD8 | 7.47E−05 | 0.499134003 | −2.00347 |
| 3110013H01RIK | 0.00068445 | 0.499134003 | −2.00347 |
| E030007N04RIK | 3.23E−07 | 0.496546519 | −2.01391 |
| PRKCD | 1.24E−06 | 0.495687519 | −2.0174 |
| 9130430L19RIK | 1.04E−05 | 0.494829037 | −2.0209 |
| 6030443O07RIK | 1.78E−05 | 0.494829037 | −2.0209 |
| A130062D16RIK | 1.86E−06 | 0.493971083 | −2.02441 |
| 5930416I19RIK | 2.26E−06 | 0.493971083 | −2.02441 |
| FYB | 9.40E−06 | 0.493971083 | −2.02441 |
| AA408556 | 0.000447224 | 0.491410151 | −2.03496 |
| TRBV31_X03277_T_CELL_RECEPTOR_BETA_VARIABLE_31_33 | 0.00870127 | 0.491410151 | −2.03496 |
| A130038J17RIK | 8.99E−05 | 0.490559188 | −2.03849 |
| AJ237586 | 1.68E−05 | 0.490559188 | −2.03849 |
| ZFP260 | 3.69E−06 | 0.490559188 | −2.03849 |
| 0710008K08RIK | 9.80E−06 | 0.489711168 | −2.04202 |
| ANP32E | 0.000183335 | 0.485486385 | −2.05979 |
| 4921518A06RIK | 8.35E−06 | 0.484644053 | −2.06337 |
| 4933421G18RIK | 1.04E−05 | 0.484644053 | −2.06337 |
| 3110018A08RIK | 0.00338526 | 0.484644053 | −2.06337 |
| C730009F21RIK | 1.18E−07 | 0.48380464 | −2.06695 |
| OLFML3 | 1.61E−05 | 0.482132181 | −2.07412 |
| A330103N21RIK | 9.39E−05 | 0.481296806 | −2.07772 |
| H2-T9 | 0.000542267 | 0.481296806 | −2.07772 |
| LOC386360 | 0.00269607 | 0.481296806 | −2.07772 |
| ILVBL | 4.13E−05 | 0.478801082 | −2.08855 |
| SBK | 5.91E−07 | 0.477972631 | −2.09217 |
| 6330403M23RIK | 6.88E−08 | 0.476319763 | −2.09943 |
| C230075L19RIK | 4.42E−07 | 0.475495347 | −2.10307 |
| MSH6 | 0.00037102 | 0.475495347 | −2.10307 |
| CXCL12 | 0.00745872 | 0.475495347 | −2.10307 |
| BC035291 | 1.11E−05 | 0.474671527 | −2.10672 |
| MMP2 | 0.000824671 | 0.473848312 | −2.11038 |
| GM525 | 0.000141229 | 0.473027946 | −2.11404 |
| STK4 | 2.73E−05 | 0.472210417 | −2.1177 |
| A130093I21RIK | 7.45E−05 | 0.471391264 | −2.12138 |
| A230013K13RIK | 3.45E−06 | 0.471391264 | −2.12138 |
| 0610041G09RIK | 0.00831324 | 0.471391264 | −2.12138 |
| TRIM28 | 3.45E−05 | 0.470577163 | −2.12505 |
| B230342M21RIK | 7.67E−06 | 0.46894857 | −2.13243 |
| 1190002H23RIK | 5.23E−05 | 0.464902208 | −2.15099 |
| TSPAN32 | 9.87E−06 | 0.462491906 | −2.1622 |
| 2610019F03RIK | 3.67E−05 | 0.462491906 | −2.1622 |
| H2-OB | 1.61E−05 | 0.461691175 | −2.16595 |
| 4732481H14RIK | 1.72E−05 | 0.460891087 | −2.16971 |
| COL5A1 | 0.00288136 | 0.460891087 | −2.16971 |
| LDH2 | 8.49E−05 | 0.459297092 | −2.17724 |
| 6720418B01RIK | 7.62E−06 | 0.458501068 | −2.18102 |
| 6430510M02RIK | 1.62E−06 | 0.458501068 | −2.18102 |
| TNFRSF7 | 0.000102955 | 0.458501068 | −2.18102 |
| CRYL1 | 8.89E−07 | 0.458501068 | −2.18102 |
| B230345P09RIK | 5.94E−05 | 0.458501068 | −2.18102 |
| CTLA4 | 0.000629703 | 0.456915183 | −2.18859 |
| RGL2 | 2.61E−06 | 0.45533401 | −2.19619 |
| 1810015C11RIK | 3.91E−09 | 0.452974457 | −2.20763 |
| F730003H07RIK | 0.00244488 | 0.452974457 | −2.20763 |
| CD97 | 0.000238915 | 0.452189956 | −2.21146 |
| LLGL1 | 1.90E−06 | 0.450625017 | −2.21914 |
| LOX | 0.000702165 | 0.450625017 | −2.21914 |
| PDXP | 3.27E−05 | 0.449844579 | −2.22299 |
| TRIB2 | 4.12E−06 | 0.449066839 | −2.22684 |
| 2210008I11RIK | 0.000393721 | 0.449066839 | −2.22684 |
| H2-AB1 | 0.00426068 | 0.449066839 | −2.22684 |
| SLC29A1 | 8.67E−06 | 0.448287765 | −2.23071 |
| ITPR2 | 5.30E−06 | 0.446737698 | −2.23845 |
| TPST1 | 9.65E−06 | 0.445964689 | −2.24233 |
| RPS6KL1 | 9.39E−07 | 0.442883526 | −2.25793 |
| RIL-PENDING | 1.64E−06 | 0.441351064 | −2.26577 |
| TTC3 | 8.83E−09 | 0.439825301 | −2.27363 |
| MAPK1 | 2.50E−07 | 0.439062514 | −2.27758 |
| H2-EB1 | 0.00320736 | 0.438302367 | −2.28153 |
| CD3D | 0.000186864 | 0.435274658 | −2.2974 |
| TRBV8_AE000663_T_CELL_RECEPTOR_BETA_VARIABLE_8_27 | 0.000766092 | 0.435274658 | −2.2974 |
| PPP1R1C | 6.89E−07 | 0.434521896 | −2.30138 |
| PITPNM2 | 5.21E−07 | 0.432267937 | −2.31338 |
| 2210419D22RIK | 1.12E−07 | 0.431519943 | −2.31739 |
| RAPGEF3 | 1.11E−07 | 0.430772677 | −2.32141 |
| SATB1 | 5.66E−06 | 0.427056598 | −2.34161 |
| C530015C18 | 9.54E−09 | 0.426317427 | −2.34567 |
| 5830496L11RIK | 1.02E−06 | 0.424843127 | −2.35381 |
| BCL7A | 3.37E−07 | 0.424843127 | −2.35381 |
| GLDC | 0.000448781 | 0.424106196 | −2.3579 |
| CD27 | 0.000101099 | 0.423371818 | −2.36199 |
| A830080H07RIK | 1.99E−05 | 0.423371818 | −2.36199 |
| ART4 | 9.75E−06 | 0.422639978 | −2.36608 |
| SCL000121.1_106 | 6.92E−06 | 0.419720131 | −2.38254 |
| 4932414K18RIK | 2.36E−06 | 0.418993828 | −2.38667 |
| ACVR2B | 4.27E−08 | 0.417543508 | −2.39496 |
| AI481316 | 4.04E−07 | 0.417543508 | −2.39496 |
| POU6F1 | 8.86E−07 | 0.417543508 | −2.39496 |
| NCK2 | 0.000270785 | 0.412508921 | −2.42419 |
| 1110046J11RIK | 5.70E−05 | 0.411082792 | −2.4326 |
| ETS2 | 1.91E−06 | 0.408243248 | −2.44952 |
| FBP1 | 0.00205479 | 0.408243248 | −2.44952 |
| TPCN1 | 3.56E−08 | 0.407536159 | −2.45377 |
| TBXA2R | 3.11E−06 | 0.405422937 | −2.46656 |
| 5430417L22RIK | 5.18E−07 | 0.404020815 | −2.47512 |
| PPARGC1B | 1.79E−07 | 0.404020815 | −2.47512 |
| TCF7 | 1.40E−05 | 0.398458762 | −2.50967 |
| DNTT | 0.00027177 | 0.397767728 | −2.51403 |
| LOC386545 | 0.00608272 | 0.394337316 | −2.5359 |
| SOX4 | 1.85E−07 | 0.390257609 | −2.56241 |
| GPR83 | 1.40E−05 | 0.388908334 | −2.5713 |
| HIBADH | 2.18E−08 | 0.387562349 | −2.58023 |
| IGH-6 | 2.87E−05 | 0.386221173 | −2.58919 |
| LOC381739 | 1.61E−06 | 0.382889437 | −2.61172 |
| DAP3 | 2.30E−08 | 0.380904496 | −2.62533 |
| DGKA | 8.26E−05 | 0.378928542 | −2.63902 |
| SNAI3 | 4.51E−07 | 0.376964456 | −2.65277 |
| SLC5A9 | 2.33E−05 | 0.376964456 | −2.65277 |
| 2410008J05RIK | 1.24E−06 | 0.376311917 | −2.65737 |
| NAV1 | 2.19E−06 | 0.376311917 | −2.65737 |
| HDAC7A | 2.22E−06 | 0.376311917 | −2.65737 |
| A130092J06RIK | 6.62E−06 | 0.375660223 | −2.66198 |
| SLA | 2.73E−05 | 0.373064727 | −2.6805 |
| MTF2 | 2.39E−06 | 0.371130501 | −2.69447 |
| C230098O21RIK | 6.12E−05 | 0.370488378 | −2.69914 |
| GFI1 | 2.05E−06 | 0.368567122 | −2.71321 |
| EPHX1 | 2.39E−08 | 0.36792977 | −2.71791 |
| BRD3 | 2.92E−06 | 0.36792977 | −2.71791 |
| AQP11 | 4.62E−07 | 0.364754502 | −2.74157 |
| IL17RB | 2.04E−07 | 0.362236156 | −2.76063 |
| RAMP1 | 0.000135079 | 0.361608725 | −2.76542 |
| NISCH | 3.31E−07 | 0.361608725 | −2.76542 |
| BGN | 0.0025721 | 0.36098216 | −2.77022 |
| TXNIP | 0.000742272 | 0.359732934 | −2.77984 |
| COL6A1 | 0.00166573 | 0.359110268 | −2.78466 |
| CCL9 | 0.000264226 | 0.356012517 | −2.80889 |
| DPP4 | 1.63E−06 | 0.354166593 | −2.82353 |
| MLL | 1.54E−07 | 0.352329781 | −2.83825 |
| C3 | 0.0001376 | 0.350503323 | −2.85304 |
| MARCKS | 4.55E−06 | 0.349896256 | −2.85799 |
| TRBV1_AE000663_T_CELL_RECEPTOR_BETA_VARIABLE_1_20 | 0.00160689 | 0.34808276 | −2.87288 |
| 3830612M24 | 2.00E−06 | 0.344481916 | −2.90291 |
| PP11R | 0.000186213 | 0.344481916 | −2.90291 |
| 2510015F01RIK | 0.000255295 | 0.342695782 | −2.91804 |
| PARD6G | 1.26E−07 | 0.327598181 | −3.05252 |
| NOTCH3 | 3.24E−06 | 0.327598181 | −3.05252 |
| H2-T10 | 0.000761011 | 0.327598181 | −3.05252 |
| LMAN2L | 1.87E−07 | 0.327031438 | −3.05781 |
| DTX1 | 2.93E−07 | 0.324772335 | −3.07908 |
| TMEM108 | 2.25E−05 | 0.324210062 | −3.08442 |
| ETS1 | 5.90E−07 | 0.322528624 | −3.1005 |
| SH2D1A | 2.22E−06 | 0.320856301 | −3.11666 |
| 9626100_15 | 0.00161165 | 0.314252942 | −3.18215 |
| CD8B | 3.35E−05 | 0.313166457 | −3.19319 |
| ACAS2L | 7.23E−06 | 0.30992664 | −3.22657 |
| LOC434197 | 1.33E−06 | 0.305659904 | −3.27161 |
| 9626100_224 | 0.00113435 | 0.305131084 | −3.27728 |
| FRAT2 | 2.01E−06 | 0.302498639 | −3.3058 |
| NRP | 8.11E−07 | 0.299888741 | −3.33457 |
| G22P1 | 8.46E−08 | 0.296273472 | −3.37526 |
| RNPEPL1 | 4.01E−09 | 0.295759985 | −3.38112 |
| 9626958_317 | 0.00188407 | 0.293717673 | −3.40463 |
| H19 | 0.000268847 | 0.283221131 | −3.53081 |
| ACTN1 | 2.58E−07 | 0.278838137 | −3.58631 |
| SLC16A5 | 3.62E−09 | 0.275476023 | −3.63008 |
| CD2 | 1.50E−06 | 0.274999519 | −3.63637 |
| PRKCB | 2.18E−07 | 0.272154758 | −3.67438 |
| ST6GAL1 | 7.38E−08 | 0.268873581 | −3.71922 |
| PRELP | 1.65E−05 | 0.268408098 | −3.72567 |
| CDCA7 | 4.57E−05 | 0.267943507 | −3.73213 |
| PDLIM4 | 4.93E−06 | 0.26701628 | −3.74509 |
| CD6 | 1.61E−09 | 0.264712734 | −3.77768 |
| ALDH2 | 7.40E−07 | 0.248703632 | −4.02085 |
| CD81 | 6.28E−06 | 0.247413906 | −4.04181 |
| 9430068D06RIK | 3.23E−10 | 0.239816205 | −4.16986 |
| H2-BL | 9.51E−06 | 0.232854352 | −4.29453 |
| AI132321 | 1.19E−06 | 0.20877219 | −4.78991 |
| LY6D | 2.50E−08 | 0.184923553 | −5.40764 |
| COX6A2 | 0.000397718 | 0.158219587 | −6.32033 |
| BCL11B | 1.64E−08 | 0.150204503 | −6.65759 |
| LOC382896 | 6.81E−09 | 0.112266584 | −8.90737 |

LAK vs. DN3

| | p-value | Ratio | Fold-Change |
|---|---|---|---|
| Column ID | (LAK vs. DN3) | (LAK vs. DN3) | (LAK vs. DN3) |
| GZMD | 2.97E−07 | 79.3411511 | 79.3413 |
| FCER1G | 8.83E−09 | 50.3005458 | 50.3005 |
| ROG | 3.50E−09 | 38.7869009 | 38.787 |
| CCL4 | 4.38E−08 | 38.5859038 | 38.5858 |
| KLRE1 | 6.58E−11 | 35.1998648 | 35.1999 |
| MT1 | 1.56E−07 | 35.0174387 | 35.0174 |
| SPP1 | 2.96E−08 | 32.5029903 | 32.503 |
| AVIL | 8.90E−08 | 30.6432634 | 30.6433 |
| TYROBP | 1.95E−10 | 26.8620793 | 26.8621 |
| GZME | 6.58E−10 | 26.1728718 | 26.1729 |
| XCL1 | 1.12E−06 | 26.1275337 | 26.1276 |
| ASB2 | 4.61E−10 | 25.8125134 | 25.8125 |
| KLRA7 | 9.89E−11 | 22.7059601 | 22.706 |
| PRF1 | 9.89E−08 | 22.2386788 | 22.2387 |
| KLRD1 | 4.72E−09 | 19.8353271 | 19.8353 |
| LGALS3 | 4.28E−09 | 18.7979112 | 18.7979 |
| GZMG | 6.92E−08 | 18.0634207 | 18.0634 |
| KLRA18 | 6.87E−08 | 16.6794542 | 16.6795 |
| SERPINA3G | 1.62E−07 | 16.5069346 | 16.5069 |
| NKG7 | 3.26E−08 | 16.4783466 | 16.4784 |
| LTB4R1 | 3.28E−07 | 16.1392364 | 16.1392 |
| GADD45G | 2.57E−08 | 16.0834021 | 16.0834 |
| CTSG | 5.88E−06 | 15.9446402 | 15.9446 |
| CCL3 | 2.43E−07 | 15.5624826 | 15.5625 |
| NFIL3 | 3.46E−07 | 15.3482283 | 15.3482 |
| AQP9 | 3.11E−07 | 15.0323722 | 15.0324 |
| 1300002F13RIK | 2.22E−07 | 14.4450222 | 14.445 |
| KLRA4 | 9.74E−08 | 14.2461507 | 14.2461 |
| LITAF | 2.42E−08 | 13.4543507 | 13.4543 |
| KLRA3 | 5.93E−09 | 13.2691108 | 13.2691 |
| LRRK1 | 1.90E−09 | 13.2232104 | 13.2232 |
| KLRG1 | 1.44E−07 | 13.2003094 | 13.2003 |
| EMILIN2 | 5.64E−06 | 12.8393439 | 12.8393 |
| 1110007C02RIK | 3.19E−11 | 12.7727787 | 12.7728 |
| TNFRSF11B | 5.09E−06 | 12.7727787 | 12.7728 |
| LOC381140 | 1.29E−08 | 12.4666362 | 12.4666 |
| HAVCR2 | 4.62E−07 | 12.0211235 | 12.0211 |
| LOC327957 | 1.39E−07 | 12.0003072 | 12.0003 |
| PDGFA | 2.78E−09 | 11.9174171 | 11.9174 |
| SCIN | 1.18E−07 | 11.7329992 | 11.733 |
| BC049975 | 2.96E−09 | 11.6519561 | 11.652 |
| PGLYRP1 | 1.73E−07 | 11.5114671 | 11.5115 |
| IFITM1 | 4.19E−05 | 11.4716392 | 11.4716 |
| SPEER3 | 4.84E−05 | 10.7405848 | 10.7406 |
| 1810044J04RIK | 3.59E−09 | 10.6848908 | 10.6849 |
| CCR5 | 1.38E−09 | 10.519539 | 10.5195 |
| WBSCR5 | 1.21E−08 | 10.4287901 | 10.4288 |
| DAF1 | 3.14E−07 | 10.3388239 | 10.3388 |
| 2210411K11RIK | 1.18E−07 | 10.091021 | 10.091 |
| P2RY14 | 3.12E−08 | 9.81508382 | 9.81508 |
| BCL2A1B | 2.31E−08 | 9.781099 | 9.78112 |
| F2R | 5.12E−07 | 9.71354748 | 9.71356 |
| LOC268288 | 1.40E−08 | 9.67997987 | 9.67995 |
| RGS1 | 1.42E−06 | 9.54672166 | 9.54669 |
| 2310057H16RIK | 5.56E−09 | 9.30171989 | 9.30174 |
| BHLHB2 | 2.26E−07 | 9.30171989 | 9.30174 |
| CTSW | 4.35E−06 | 9.07877654 | 9.07879 |
| 5330403J18RIK | 6.45E−09 | 9.06306078 | 9.06307 |
| SH2D1B1 | 3.54E−07 | 8.89197144 | 8.69195 |
| PLSCR1 | 1.15E−10 | 8.86116329 | 8.86119 |
| 1110018K11RIK | 7.85E−11 | 8.69391339 | 8.69388 |
| ICSBP1 | 4.16E−07 | 8.58907298 | 8.58906 |
| SPEER1-PS1 | 0.000100291 | 8.29648312 | 8.29648 |
| RGS16 | 6.08E−08 | 8.22490171 | 8.22491 |
| EG433016 | 0.000986707 | 8.2106525 | 8.21067 |
| DHRS6 | 2.02E−06 | 8.19645258 | 8.19646 |
| 2310067E08RIK | 7.56E−08 | 7.88985759 | 7.88986 |
| KLRA13 | 3.45E−10 | 7.87618635 | 7.8762 |
| TNFSF6 | 7.56E−09 | 7.70077855 | 7.70076 |
| CCL5 | 0.000147502 | 7.38705198 | 7.38706 |
| E030006K04RIK | 2.60E−09 | 7.23505238 | 7.23503 |
| CAR2 | 7.88E−11 | 6.90431312 | 6.90432 |
| SERPINE2 | 1.33E−06 | 6.90431312 | 6.90432 |
| IER3 | 7.22E−06 | 6.84476752 | 6.84476 |
| TNFRSF9 | 1.74E−08 | 6.83288806 | 6.83291 |
| SIAT10 | 6.93E−07 | 6.7974958 | 6.79748 |
| GLRX1 | 4.58E−06 | 6.64606387 | 6.64606 |
| DAPK2 | 7.72E−10 | 6.61161396 | 6.6116 |
| F2RL2 | 5.36E−06 | 6.55458329 | 6.55456 |
| IFITM3 | 2.68E−06 | 6.46433304 | 6.46433 |
| MLKL | 5.34E−09 | 6.30938711 | 6.30939 |
| SYTL2 | 1.70E−10 | 6.14749059 | 6.1475 |
| TMEM119 | 0.000650503 | 6.1368518 | 6.13686 |
| 2810025M15RIK | 1.47E−08 | 6.0418945 | 6.04189 |
| SEPN1 | 8.40E−07 | 6.0418945 | 6.04189 |
| TCRD-V1 | 7.87E−09 | 6.01055453 | 6.01056 |
| OSBPL3 | 1.46E−11 | 5.98978143 | 5.98977 |
| APOB48R | 3.32E−07 | 5.98978143 | 5.98977 |
| CD52 | 2.73E−05 | 5.979395 | 5.9794 |
| MYO1F | 2.88E−09 | 5.93810123 | 5.93809 |
| SH3BP2 | 4.75E−09 | 5.8970609 | 5.89706 |
| RASD2 | 1.15E−06 | 5.85634388 | 5.85634 |
| LOC269941 | 3.60E−10 | 5.80581859 | 5.80582 |
| IFITM2 | 6.30E−05 | 5.80581859 | 5.80582 |
| GPR87 | 1.21E−06 | 5.77570622 | 5.77572 |
| LOC270152 | 2.10E−06 | 5.74577255 | 5.74577 |
| HIST1H1C | 2.95E−05 | 5.71598418 | 5.71598 |
| AA467197 | 3.72E−05 | 5.70606897 | 5.70608 |
| KLRA1 | 3.83E−07 | 5.63729635 | 5.63728 |
| IDB2 | 1.24E−08 | 5.55003635 | 5.55005 |
| LY6A | 2.01E−05 | 5.54044246 | 5.54044 |
| FCGR3 | 2.61E−07 | 5.51170687 | 5.51171 |
| GVIN1 | 3.28E−05 | 5.51170687 | 5.51171 |
| A430038C16RIK | 2.04E−08 | 5.42640707 | 5.42642 |
| ID2 | 4.68E−09 | 5.37961235 | 5.3796 |
| S100A1 | 3.25E−08 | 5.37961235 | 5.3796 |
| CAPG | 1.77E−09 | 5.37028087 | 5.37029 |
| PPP3CC | 8.68E−10 | 5.33319111 | 5.33319 |
| 1500031H04RIK | 2.19E−07 | 5.30554642 | 5.30554 |
| PLCG2 | 3.91E−09 | 5.25978056 | 5.25977 |
| NCF4 | 1.99E−07 | 5.24158464 | 5.24157 |
| AI115600 | 1.22E−09 | 5.14260442 | 5.14261 |
| DUSP6 | 8.20E−10 | 5.11595308 | 5.11594 |
| SERPINB6A | 9.97E−06 | 5.09824315 | 5.09824 |
| BCL2A1D | 1.37E−08 | 4.89905497 | 4.89904 |
| UPP1 | 1.21E−08 | 4.85677374 | 4.85678 |
| PIM3 | 1.14E−06 | 4.85677374 | 4.85678 |
| AMICA1 | 1.15E−06 | 4.84836731 | 4.84837 |
| SLC2A3 | 4.16E−06 | 4.8148915 | 4.81488 |
| 5031436O0RIK | 2.13E−06 | 4.77333804 | 4.77334 |
| CD160 | 1.84E−05 | 4.73215976 | 4.73216 |
| A430084P05RIK | 2.72E−07 | 4.6913337 | 4.69134 |
| GOLPH2 | 1.25E−08 | 4.61874564 | 4.61874 |
| CD244 | 8.49E−09 | 4.61874564 | 4.61874 |
| DMWD | 1.23E−06 | 4.57890134 | 4.5789 |
| AHNAK | 1.67E−08 | 4.57096886 | 4.57097 |
| TRAF1 | 2.12E−06 | 4.57096886 | 4.57097 |
| TES | 1.82E−08 | 4.55514458 | 4.55515 |
| CDKN2B | 1.88E−05 | 4.55514458 | 4.55515 |
| 1110004P15RIK | 6.74E−06 | 4.55514458 | 4.55515 |
| SRGAP2 | 1.33E−08 | 4.50803783 | 4.50804 |
| SULF2 | 3.60E−09 | 4.4691539 | 4.46915 |
| HHEX | 1.23E−08 | 4.38477256 | 4.38477 |
| LAG3 | 5.75E−07 | 4.30198192 | 4.30198 |
| ALDOA | 1.30E−05 | 4.22074496 | 4.22075 |
| AI850995 | 5.34E−08 | 4.2134358 | 4.21344 |
| PTER | 3.14E−06 | 4.20615192 | 4.20615 |
| LOC218482 | 9.08E−09 | 4.19887554 | 4.19887 |
| 1190002C06RIK | 2.68E−07 | 4.16986356 | 4.16986 |
| CTNNA1 | 3.18E−08 | 4.15542969 | 4.15544 |
| CDKN1A | 0.000116956 | 4.14106111 | 4.14106 |
| 2310016C16RIK | 9.43E−06 | 4.12674045 | 4.12673 |
| A530050E01RIK | 2.79E−07 | 4.08404974 | 4.08405 |
| LMNA | 1.14E−06 | 4.06287709 | 4.06287 |
| GLIPR1 | 1.07E−07 | 4.03481236 | 4.03481 |
| 9830144J08RIK | 4.06E−09 | 4.02084406 | 4.02085 |
| PPAP2C | 3.54E−07 | 3.95862446 | 3.95863 |
| SAT1 | 1.91E−05 | 3.95862446 | 3.95863 |
| SLC2A1 | 5.83E−05 | 3.95862446 | 3.95863 |
| HAK | 4.36E−08 | 3.93809318 | 3.9381 |
| SCL0003187.1_40 | 6.01E−07 | 3.91768167 | 3.91768 |
| 2310046K01RIK | 7.02E−06 | 3.86374876 | 3.86375 |
| RHOF | 3.34E−07 | 3.84371457 | 3.84371 |
| LOC212399 | 4.55E−08 | 3.82378471 | 3.82378 |
| CD69 | 3.83E−05 | 3.81055447 | 3.51055 |
| B230343A10RIK | 1.49E−08 | 3.80395916 | 3.80395 |
| BC022224 | 7.94E−08 | 3.7776762 | 3.77768 |
| KLRB1C | 9.87E−09 | 3.74509393 | 3.74509 |
| MYO1E | 8.79E−09 | 3.7192142 | 3.71922 |
| KLRA33 | 3.61E−06 | 3.69352599 | 3.69353 |
| WDFY1 | 5.95E−08 | 3.68074675 | 3.68075 |
| GCNT1 | 5.12E−08 | 3.64268203 | 3.64268 |
| C80638 | 3.80E−05 | 3.64268203 | 3.64268 |
| HGFAC | 7.89E−05 | 3.63637686 | 3.63637 |
| RASL12 | 4.28E−07 | 3.61125556 | 3.61125 |
| GNG2 | 1.28E−09 | 3.58009752 | 3.5801 |
| SH2D2A | 1.04E−06 | 3.56770796 | 3.56771 |
| GPR141 | 1.94E−05 | 3.56770796 | 3.56771 |
| DCI | 2.41E−09 | 3.51859932 | 3.5186 |
| GLRX | 4.20E−05 | 3.51859932 | 3.5186 |
| LOC385699 | 9.48E−07 | 3.51250628 | 3.5125 |
| CD72 | 8.84E−05 | 3.48824635 | 3.48824 |
| EGR1 | 0.000778832 | 3.48824635 | 3.48824 |
| 2210008N01RIK | 1.43E−08 | 3.45814948 | 3.45815 |
| ADORA2B | 6.64E−07 | 3.44618438 | 3.44618 |
| KLRA10 | 4.19E−08 | 3.43426642 | 3.43426 |
| S100A11 | 2.42E−07 | 3.43426642 | 3.43426 |
| F730045P10RIK | 3.27E−05 | 3.43426642 | 3.43426 |
| PILRB | 4.70E−05 | 3.42832067 | 3.42832 |
| LGALS1 | 2.90E−06 | 3.4046378 | 3.40463 |
| S100A10 | 7.33E−06 | 3.39285533 | 3.39286 |
| NQO2 | 4.88E−08 | 3.38698315 | 3.38698 |
| CD9 | 1.56E−07 | 3.38111983 | 3.38112 |
| PLP2 | 5.92E−08 | 3.36941982 | 3.36942 |
| SLC39A4 | 3.40E−08 | 3.35194782 | 3.35195 |
| 7420404O03RIK | 2.40E−06 | 3.33456712 | 3.33457 |
| NEDD9 | 3.25E−07 | 3.31153241 | 3.31153 |
| BSPRY | 1.11E−06 | 3.28866234 | 3.28866 |
| STX11 | 2.41E−06 | 3.28866234 | 3.28866 |
| NFKBIZ | 5.41E−07 | 3.27728091 | 3.27728 |
| ADAM8 | 1.09E−06 | 3.27728091 | 3.27728 |
| PPP1R3B | 1.39E−06 | 3.27728091 | 3.27728 |
| SEC61B | 1.17E−10 | 3.26594598 | 3.26594 |
| S100A6 | 4.92E−06 | 3.26594598 | 3.26594 |
| ANXA2 | 4.93E−06 | 3.25464682 | 3.25464 |
| HK2 | 1.70E−06 | 3.19319466 | 3.19319 |
| PDLIM7 | 9.37E−08 | 3.18766755 | 3.18766 |
| TRIO | 4.54E−07 | 3.17664026 | 3.17664 |
| ENO1 | 2.54E−07 | 3.16016673 | 3.16017 |
| LOC383981 | 0.000359161 | 3.15469355 | 3.15469 |
| N4WBP5-PENDING | 5.19E−09 | 3.14922938 | 3.14923 |
| LOC328703 | 2.98E−07 | 3.14378411 | 3.14378 |
| HIC1 | 5.07E−06 | 3.11666293 | 3.11666 |
| AOAH | 2.89E−09 | 3.11126183 | 3.11126 |
| 1110020C13R1K | 2.86E−08 | 3.11126183 | 3.11126 |
| MAPKAPK3 | 2.85E−07 | 3.10587943 | 3.10588 |
| FTL1 | 8.72E−06 | 3.09513198 | 3.09513 |
| BC046404 | 6.62E−07 | 3.07908317 | 3.07908 |
| E030003N15RIK | 8.70E−06 | 3.07908317 | 3.07908 |
| VIM | 1.02E−05 | 3.0684259 | 3.06843 |
| CISH | 1.19E−05 | 3.0684259 | 3.06843 |
| C230043G09RIK | 1.82E−07 | 3.05252169 | 3.05252 |
| GPR97 | 4.15E−06 | 3.04195464 | 3.04196 |
| LASP1 | 1.17E−07 | 3.03668928 | 3.03669 |
| 9930117H01RIK | 6.85E−06 | 3.03668928 | 3.03669 |
| CASP1 | 0.000296727 | 3.000075 | 3.00008 |
| D10ERTD438E | 3.40E−07 | 2.97935604 | 2.97935 |
| TBC1D2B | 7.23E−09 | 2.97419883 | 2.9742 |
| 1700129I15RIK | 2.83E−05 | 2.96390263 | 2.96391 |
| NDFIP1 | 1.87E−07 | 2.95877246 | 2.95878 |
| 5730469M10RIK | 9.16E−05 | 2.95877246 | 2.95878 |
| JUNB | 0.000168611 | 2.95877246 | 2.95878 |
| PGK1 | 2.49E−06 | 2.9536513 | 2.95365 |
| LOC240672 | 1.77E−07 | 2.94853915 | 2.94854 |
| BSCL2 | 2.21E−06 | 2.94343599 | 2.94343 |
| IFNG | 1.45E−05 | 2.9383332 | 2.93834 |
| S100A13 | 6.20E−05 | 2.93324807 | 2.93325 |
| CHN2 | 1.49E−07 | 2.92817194 | 2.92817 |
| CST7 | 1.31E−07 | 2.9231048 | 2.9231 |
| POLD4 | 1.30E−05 | 2.91298902 | 2.91299 |
| BC087945 | 2.69E−07 | 2.90794888 | 2.90794 |
| 4631423F02RIK | 2.76E−05 | 2.89286561 | 2.89287 |
| 2310061N23RIK | 0.00286637 | 2.87288627 | 2.87288 |
| GIPC2 | 3.20E−05 | 2.86294236 | 2.86295 |
| AA175286 | 9.62E−05 | 2.84810217 | 2.8481 |
| TUBA6 | 4.58E−07 | 2.83824823 | 2.83825 |
| KLRB1D | 7.14E−11 | 2.83333475 | 2.83333 |
| HRMT1L1 | 2.71E−07 | 2.83333475 | 2.83333 |
| TMEM126A | 2.96E−08 | 2.82843025 | 2.82843 |
| TRF | 3.44E−06 | 2.81864023 | 2.81864 |
| LOC383189 | 2.82E−08 | 2.81376268 | 2.81376 |
| AW536289 | 4.33E−07 | 2.81376268 | 2.81376 |
| CCNG1 | 1.58E−05 | 2.81376268 | 2.81376 |
| HAAO | 5.90E−06 | 2.79916809 | 2.79917 |
| 6720467C03RIK | 2.29E−11 | 2.7943264 | 2.79433 |
| SLC24A3 | 3.28E−05 | 2.7948587 | 2.78949 |
| PSTPIP1 | 5.32E−06 | 2.77502588 | 2.77502 |
| TBX21 | 4.36E−06 | 2.76542554 | 2.76542 |
| PRDX5 | 7.28E−05 | 2.76542554 | 2.76542 |
| EGR3 | 0.000564399 | 2.76063119 | 2.76063 |
| TFF1 | 0.00864278 | 2.76063119 | 2.76063 |
| E130012A19RIK | 7.32E−06 | 2.74631719 | 2.74632 |
| BCAP29 | 8.13E−06 | 2.74631719 | 2.74632 |
| IRAK2 | 2.06E−05 | 2.74156626 | 2.74157 |
| 2310016C08RIK | 0.000897768 | 2.74156626 | 2.74157 |
| E430036I04RIK | 9.77E−08 | 2.73681675 | 2.73682 |
| FES | 1.41E−07 | 2.73208368 | 2.73208 |
| C330023F11RIK | 1.27E−07 | 2.71791568 | 2.71791 |
| FKBP11 | 0.000163935 | 2.7132109 | 2.71321 |
| GP49A | 8.69E−07 | 2.70850769 | 2.70851 |
| FHL2 | 3.43E−08 | 2.70382077 | 2.70382 |
| PRMT2 | 5.08E−07 | 2.69446637 | 2.69447 |
| 3300005D01RIK | 5.96E−05 | 2.68514764 | 2.68514 |
| RPS6KA1 | 5.06E−08 | 2.66659556 | 2.6666 |
| GPD2 | 2.77E−08 | 2.66198158 | 2.66198 |
| SNAG1 | 8.24E−08 | 2.66198158 | 2.66198 |
| CLN3 | 2.04E−05 | 2.65277321 | 2.65277 |
| TMPIT | 4.29E−07 | 2.64817685 | 2.64816 |
| 1810011E08RIK | 4.07E−06 | 2.64817885 | 2.64818 |
| BIN1 | 6.15E−08 | 2.64359338 | 2.64359 |
| PEA15 | 3.62E−08 | 2.62988336 | 2.62989 |
| SDF2L1 | 4.71E−05 | 2.62988336 | 2.62989 |
| LOC383099 | 3.70E−05 | 2.62988336 | 2.62989 |
| 4930486L24RIK | 2.78E−06 | 2.60719481 | 2.6072 |
| CAMK2N1 | 2.06E−06 | 2.60268232 | 2.60266 |
| IFI30 | 2.03E−06 | 2.59817868 | 2.59818 |
| 4930513E20RIK | 0.000992688 | 2.59367713 | 2.59368 |
| 0610037M15RIK | 7.56E−05 | 2.58919116 | 2.58919 |
| PFKP | 1.61E−05 | 2.58470732 | 2.58471 |
| A630024B12RIK | 2.56E−06 | 2.58023232 | 2.58023 |
| SPIN2 | 4.24E−07 | 2.57576616 | 2.57576 |
| MMD | 1.33E−06 | 2.57576616 | 2.57576 |
| MGC18837 | 1.51E−06 | 2.56240743 | 2.56241 |
| C130027E04RIK | 3.69E−07 | 2.55354138 | 2.55354 |
| IL18R1 | 5.54E−06 | 2.55354138 | 2.55354 |
| GALGT1 | 1.55E−05 | 2.55354138 | 2.55354 |
| STK39 | 3.36E−08 | 2.54912157 | 2.54912 |
| OBFC2A | 8.44E−06 | 2.54912157 | 2.54912 |
| D930046M13RIK | 1.92E−05 | 2.54471056 | 2.54471 |
| ALDOC | 0.00134083 | 2.54471056 | 2.54471 |
| IL2RB | 4.01E−07 | 2.54030189 | 2.5403 |
| LOC381319 | 0.000586562 | 2.53151098 | 2.53151 |
| BAG3 | 0.00125353 | 2.53151098 | 2.53151 |
| DOK2 | 1.17E−06 | 2.52275525 | 2.52275 |
| UGCG | 8.13E−05 | 2.52275525 | 2.52275 |
| ARRDC4 | 0.000487201 | 2.5183842 | 2.51839 |
| ATF4 | 0.00225306 | 2.5183842 | 2.51839 |
| IL12RB1 | 6.73E−07 | 2.51402828 | 2.51403 |
| 9130211I03RIK | 0.000664109 | 2.51402828 | 2.51403 |
| 1810061M12RIK | 7.55E−08 | 2.50533009 | 2.50533 |
| KLRA21 | 1.66E−06 | 2.50099415 | 2.50099 |
| MVP | 3.59E−07 | 2.49666072 | 2.49666 |
| CYBA | 1.03E−06 | 2.49233607 | 2.49234 |
| BATF | 2.83E−06 | 2.49233607 | 2.49234 |
| NENF | 1.16E−05 | 2.49233607 | 2.49234 |
| TNFSF13 | 2.10E−08 | 2.48802018 | 2.48802 |
| EHD4 | 1.57E−05 | 2.48802018 | 2.48802 |
| CSDA | 3.96E−08 | 2.47941466 | 2.47942 |
| CRELD2 | 1.86E−05 | 2.47512499 | 2.47512 |
| LOC238943 | 2.92E−06 | 2.47083794 | 2.47084 |
| 1110019C08RIK | 3.97E−05 | 2.47083794 | 2.47084 |
| OSTF1 | 8.10E−08 | 2.46655962 | 2.46656 |
| 2510048K03RIK | 4.28E−07 | 2.46655962 | 2.46656 |
| 1700025G04RIK | 1.15E−05 | 2.46229003 | 2.46229 |
| PADI2 | 1.20E−08 | 2.45802311 | 2.45803 |
| A530060O05RIK | 9.89E−07 | 2.45802311 | 2.45803 |
| FXYD5 | 9.20E−06 | 2.45802311 | 2.45803 |
| MYO1G | 6.72E−06 | 2.45377096 | 2.45377 |
| ECH1 | 1.99E−05 | 2.45377096 | 2.45377 |
| GABARAPL1 | 5.14E−09 | 2.44528073 | 2.44528 |
| AI847670 | 4.70E−07 | 2.44104867 | 2.44105 |
| PPIB | 3.62E−09 | 2.43681937 | 2.43682 |
| FOSL2 | 1.02E−05 | 2.42839277 | 2.42839 |
| BC023892 | 3.65E−09 | 2.42418366 | 2.42419 |
| STX7 | 1.05E−06 | 2.42418366 | 2.42419 |
| NUCB1 | 1.57E−05 | 2.42418366 | 2.42419 |
| KLF7 | 1.89E−05 | 2.42418366 | 2.42419 |
| TAF9B | 8.39E−07 | 2.41998911 | 2.41999 |
| LOC381683 | 4.83E−07 | 2.41998911 | 2.41999 |
| LOC386405 | 0.00419371 | 2.41998911 | 2.41999 |
| H47 | 6.51E−07 | 2.41579738 | 2.4158 |
| GPR34 | 1.34E−07 | 2.41161433 | 2.41162 |
| 1110006I15RIK | 1.08E−07 | 2.40743995 | 2.40744 |
| MYL6 | 2.17E−08 | 2.40327422 | 2.40327 |
| SOAT2 | 1.66E−05 | 2.40327422 | 2.40327 |
| HIP1 | 1.04E−10 | 2.39911137 | 2.39911 |
| DEGS | 5.19E−07 | 2.39911137 | 2.39911 |
| 2810032E02RIK | 1.12E−06 | 2.39911137 | 2.39911 |
| SH3BGRL3 | 0.000169298 | 2.39911137 | 2.39911 |
| DTR | 4.75E−06 | 2.38666902 | 2.38667 |
| SLK | 1.18E−08 | 2.38254074 | 2.38254 |
| EOMES | 8.89E−07 | 2.3784154 | 2.37841 |
| GMDS | 2.61E−06 | 2.3784154 | 2.37841 |
| DCXR | 3.22E−05 | 2.37018497 | 2.37019 |
| H2-Q8 | 0.00206753 | 2.37018497 | 2.37019 |
| HIP-1 | 1.62E−10 | 2.36607988 | 2.36608 |
| DAB2IP | 5.55E−08 | 2.36607988 | 2.36608 |
| KLRK1 | 3.04E−08 | 2.36607988 | 2.36608 |
| OLFM1 | 7.39E−08 | 2.3619834 | 2.36199 |
| CABLES1 | 2.23E−06 | 2.3619834 | 2.36199 |
| AI840980 | 0.000110731 | 2.3619834 | 2.36199 |
| BC024955 | 4.20E−08 | 2.35789553 | 2.3579 |
| MINA | 4.70E−09 | 2.35789553 | 2.3579 |
| A530090P03RIK | 6.45E−06 | 2.35789553 | 2.3579 |
| NRGN | 4.73E−05 | 2.3538107 | 2.35381 |
| ZFP52 | 0.000839487 | 2.3538107 | 2.35381 |
| TSPO | 2.07E−06 | 2.34974 | 2.34974 |
| TDRD7 | 2.88E−06 | 2.34567235 | 2.34567 |
| TMEM38B | 6.08E−08 | 2.34160779 | 2.34161 |
| SAMSN1 | 1.16E−06 | 2.34160779 | 2.34161 |
| IAN4 | 0.000116004 | 2.34160779 | 2.34161 |
| IMPA2 | 0.000464842 | 2.33755184 | 2.33755 |
| 2310056P07RIK | 0.000374894 | 2.33755184 | 2.33755 |
| ETFB | 8.23E−07 | 2.3335099 | 2.33351 |
| GZMK | 0.000514646 | 2.32140826 | 2.32141 |
| 5730438N18RIK | 1.67E−05 | 2.31738969 | 2.31739 |
| LOC215678 | 4.32E−05 | 2.31337431 | 2.31338 |
| LOC269355 | 1.94E−05 | 2.30937282 | 2.30937 |
| STK32C | 4.69E−07 | 2.30537452 | 2.30537 |
| SLAMF7 | 1.11E−06 | 2.30537452 | 2.30537 |
| ABCB1B | 1.12E−06 | 2.30137945 | 2.30138 |
| 4930539E08RIK | 3.24E−05 | 2.30137945 | 2.30138 |
| CLNK | 3.67E−08 | 2.2973982 | 2.2974 |
| TEX9 | 1.16E−06 | 2.2973982 | 2.2974 |
| LOC218617 | 6.06E−08 | 2.29342018 | 2.29342 |
| PALD | 8.48E−07 | 2.29342018 | 2.29342 |
| GPR114 | 0.00411637 | 2.29342018 | 2.29342 |
| GOLGA7 | 1.77E−06 | 2.28945067 | 2.28945 |
| GPR160 | 5.41E−06 | 2.28945067 | 2.28945 |
| KIT | 0.00014555 | 2.28945067 | 2.28945 |
| SQSTM1 | 0.000722465 | 2.28945067 | 2.28945 |
| KLRA16 | 3.18E−07 | 2.28548443 | 2.28548 |
| ZBTB32 | 1.19E−05 | 2.28548443 | 2.28548 |
| 1110030J09RIK | 5.87E−10 | 2.28152671 | 2.28153 |
| CYP51 | 2.62E−06 | 2.28152671 | 2.28153 |
| 3110054C06RIK | 1.87E−06 | 2.28152671 | 2.28153 |
| C730026J16 | 4.19E−05 | 2.26969929 | 2.2697 |
| SERTAD1 | 7.39E−05 | 2.26969929 | 2.2697 |
| 2310004N11RIK | 8.08E−05 | 2.26969929 | 2.2697 |
| VEGFC | 1.79E−07 | 2.26576519 | 2.26577 |
| LOC114601 | 7.89E−06 | 2.26576519 | 2.26577 |
| A930008A22RIK | 1.15E−06 | 2.26184472 | 2.26184 |
| GFOD1 | 6.22E−07 | 2.26184472 | 2.26184 |
| STK2 | 7.89E−09 | 2.25011869 | 2.25012 |
| BC036961 | 4.29E−06 | 2.25011869 | 2.25012 |
| 1810006K23RIK | 4.62E−06 | 2.25011869 | 2.25012 |
| 2310047C17RIK | 0.00113428 | 2.25011869 | 2.25012 |
| CAI | 2.16E−07 | 2.23844849 | 2.23845 |
| CAPNS1 | 1.49E−08 | 2.23457196 | 2.23457 |
| RPL36 | 3.70E−05 | 2.23457196 | 2.23457 |
| 2310043N10RIK | 7.41E−05 | 2.23070385 | 2.23071 |
| SYPL | 1.01E−06 | 2.22684416 | 2.22684 |
| GZMN | 0.00220233 | 2.22684416 | 2.22684 |
| COMT | 6.85E−09 | 2.22298792 | 2.22299 |
| SCL000416.1_19 | 1.60E−08 | 2.22298792 | 2.22299 |
| LOC382127 | 0.000238522 | 2.22298792 | 2.22299 |
| 2610009E16RIK | 0.000238786 | 2.21914008 | 2.21914 |
| GPC1 | 0.0145298 | 2.21914008 | 2.21914 |
| ASAH1 | 2.95E−08 | 2.21145978 | 2.21146 |
| HADH2 | 7.87E−06 | 2.21145978 | 2.21146 |
| XDH | 1.37E−06 | 2.20763223 | 2.20763 |
| DP1 | 5.63E−06 | 2.20380818 | 2.20381 |
| TGFBR2 | 3.17E−07 | 2.19999252 | 2.19999 |
| SC4MOL | 1.44E−06 | 2.19999252 | 2.19999 |
| XAB1 | 1.99E−06 | 2.19999252 | 2.19999 |
| PTPN8 | 3.84E−06 | 2.19238147 | 2.19238 |
| DIP3B | 3.01E−06 | 2.18479904 | 2.1848 |
| VTI1B | 8.79E−06 | 2.18479904 | 2.1848 |
| STK17B | 1.46E−05 | 2.18479904 | 2.1848 |
| GNS | 0.000892576 | 2.18479904 | 2.1848 |
| MTMR9 | 0.00171537 | 2.18479904 | 2.1848 |
| ALAD | 2.13E−08 | 2.18101557 | 2.18102 |
| ARPC1B | 3.71E−07 | 2.18101557 | 2.18102 |
| 2600010E01RIK | 1.17E−05 | 2.18101557 | 2.18102 |
| LOC237361 | 1.67E−05 | 2.15845662 | 2.15846 |
| MPP6 | 2.77E−06 | 2.15471732 | 2.15472 |
| PDCD1LG2 | 8.02E−06 | 2.15471732 | 2.15472 |
| SERPINB6B | 2.58E−05 | 2.15471732 | 2.15472 |
| CAPN2 | 1.74E−06 | 2.15099096 | 2.15099 |
| ELOVL1 | 2.29E−06 | 2.15099096 | 2.15099 |
| RAB3D | 2.65E−05 | 2.15099096 | 2.15099 |
| HINT2 | 0.000350321 | 2.14726363 | 2.14726 |
| FIGF | 4.55E−05 | 2.1435492 | 2.14355 |
| OSM | 0.000887713 | 2.1435492 | 2.14355 |
| ACATE3 | 1.20E−06 | 2.13983386 | 2.13984 |
| 5430427O19RIK | 3.35E−06 | 2.13983386 | 2.13984 |
| EG630499 | 0.00033688 | 2.12874206 | 2.12874 |
| KLK1B11 | 0.00366553 | 2.12874206 | 2.12874 |
| FNBP1 | 3.25E−08 | 2.12137669 | 2.12138 |
| SRI | 2.09E−08 | 2.12137669 | 2.12138 |
| 2610529H08RIK | 2.11E−07 | 2.12137669 | 2.12138 |
| ANXA5 | 0.000191309 | 2.12137669 | 2.12138 |
| RPIA | 2.24E−08 | 2.11403529 | 2.11404 |
| LCP1 | 1.15E−05 | 2.11403529 | 2.11404 |
| LOC23352 | 3.13E−05 | 2.11037693 | 2.11038 |
| UGALT2 | 1.37E−05 | 2.10672234 | 2.10672 |
| ANXA3 | 0.00588432 | 2.10307596 | 2.10307 |
| JAM4 | 2.28E−07 | 2.09943336 | 2.09943 |
| CHST12 | 6.51E−07 | 2.09943336 | 2.09943 |
| SCL0001297.1_42 | 3.97E−05 | 2.09943336 | 2.09943 |
| PYGL | 9.38E−05 | 2.09943336 | 2.09943 |
| MREG | 1.19E−07 | 2.09579897 | 2.0958 |
| 1810003N24RIK | 2.62E−06 | 2.09579897 | 2.0958 |
| HRC | 0.000100933 | 2.09579897 | 2.0958 |
| DDIT4 | 0.0101176 | 2.09216839 | 2.09217 |
| FBXO4 | 2.51E−07 | 2.08493178 | 2.08493 |
| 201007E07RIK | 5.45E−06 | 2.08493178 | 2.08493 |
| ZFP296 | 2.06E−08 | 2.08132139 | 2.08132 |
| 0610039D01RIK | 7.86E−07 | 2.08132139 | 2.08132 |
| COTL1 | 0.000383267 | 2.08132139 | 2.08132 |
| KLRI1 | 1.30E−08 | 2.07771916 | 2.07772 |
| UBL4 | 2.81E−07 | 2.07771916 | 2.07772 |
| ARHGAP18 | 3.33E−07 | 2.07771916 | 2.07772 |
| SNX9 | 1.75E−07 | 2.07412078 | 2.07412 |
| PFN1 | 3.03E−07 | 2.07412078 | 2.07412 |
| 9130227C08RIK | 2.98E−06 | 2.07412078 | 2.07412 |
| DAP | 2.54E−05 | 2.07412078 | 2.07412 |
| 9030611O19RIK | 7.73E−05 | 2.07412078 | 2.07412 |
| D8ERTD354E | 0.000213556 | 2.07412078 | 2.07412 |
| 2900026A02RIK | 3.15E−07 | 2.07053055 | 2.07053 |
| M6PR | 1.54E−06 | 2.07053055 | 2.07053 |
| MRPS6 | 1.82E−05 | 2.07053055 | 2.07053 |
| 0610009O03RIK | 1.91E−08 | 2.06694419 | 2.06695 |
| CAPZB | 4.50E−07 | 2.06336597 | 2.06337 |
| ARRB2 | 1.28E−06 | 2.06336597 | 2.06337 |
| A430093B03RIK | 4.59E−07 | 2.05979587 | 2.05979 |
| NIBAN | 3.10E−05 | 2.05979587 | 2.05979 |
| 2610036L11RIK | 0.00147553 | 2.05979587 | 2.05979 |
| DHRS7 | 1.18E−08 | 2.05622966 | 2.05623 |
| LOC241621 | 3.38E−06 | 2.05266734 | 2.05267 |
| COX7A1 | 0.000599505 | 2.05266734 | 2.05267 |
| RBMS1 | 2.03E−07 | 2.04911314 | 2.04911 |
| CDKN2A | 0.000238387 | 2.04911314 | 2.04911 |
| BB220380 | 1.50E−07 | 2.03849078 | 2.03849 |
| CMKBR2 | 3.22E−06 | 2.03849078 | 2.03849 |
| AW212394 | 2.59E−05 | 2.03849078 | 2.03849 |
| 1110030C22RIK | 0.000169536 | 2.03496062 | 2.03496 |
| SCL00319622.1_241 | 8.06E−06 | 2.03143442 | 2.03144 |
| LCN4 | 0.00307845 | 2.03143442 | 2.03144 |
| KDELR2 | 1.28E−06 | 2.02792041 | 2.02792 |
| CD59A | 0.00082023 | 2.02440624 | 2.02441 |
| CAPN5 | 0.00123881 | 2.01740211 | 2.0174 |
| ZFP608 | 2.50E−06 | 2.0139121 | 2.01391 |
| SLC2A6 | 0.000147228 | 2.0139121 | 2.01391 |
| CORO1C | 1.61E−06 | 2.0139121 | 2.01391 |
| GNPDA1 | 7.70E−07 | 2.0139121 | 2.01391 |
| CARD4 | 8.05E−06 | 2.01042607 | 2.01042 |
| 09-Sep | 2.79E−06 | 2.00694403 | 2.00694 |
| 2410012H22RIK | 8.81E−07 | 2.00347001 | 2.00347 |
| SKAP2 | 1.02E−06 | 2.00347001 | 2.00347 |
| IAN3 | 0.0043871 | 2.00347001 | 2.00347 |
| TPI1 | 0.00037172 | 2.00347001 | 2.00347 |
| 9130422G05RIK | 8.52E−08 | 2 | 2 |
| 2810004N20RIK | 1.13E−06 | 2 | 2 |
| B4GALNT2 | 0.00262346 | 2 | 2 |
| DNMT3B | 1.05E−06 | 0.5 | −2 |
| SCL000548.1_6 | 2.96E−06 | 0.5 | −2 |
| LYT-2 | 2.32E−05 | 0.5 | −2 |
| LBR | 1.18E−05 | 0.499134 | −2.00347 |
| 6330406L22RIK | 5.22E−05 | 0.499134 | −2.00347 |
| MIER1 | 1.68E−08 | 0.498271 | −2.00694 |
| 1810020D17RIK | 2.21E−07 | 0.498271 | −2.00694 |
| SLC9A9 | 4.02E−05 | 0.498271 | −2.00694 |
| TCRG-V5 | 0.00106772 | 0.498271 | −2.00694 |
| BC035295 | 8.94E−08 | 0.4974085 | −2.01042 |
| LOC386192 | 0.0045284 | 0.4974085 | −2.01042 |
| ARHGEF11 | 2.19E−08 | 0.49654652 | −2.01391 |
| B230114J08RIK | 6.30E−07 | 0.49654652 | −2.01391 |
| ARID1A | 2.03E−06 | 0.49654652 | −2.01391 |
| C030002B11RIK | 3.75E−05 | 0.49654652 | −2.01391 |
| E430013K19RIK | 3.08E−06 | 0.49568752 | −2.0174 |
| A130022A09RIK | 4.53E−06 | 0.49568752 | −2.0174 |
| LOC269401 | 5.58E−06 | 0.49568752 | −2.0174 |
| 2700007B13RIK | 3.11E−05 | 0.49568752 | −2.0174 |
| DDAH1 | 0.000139319 | 0.49568752 | −2.0174 |
| HP | 0.00181193 | 0.49568752 | −2.0174 |
| DNCHC1 | 5.02E−06 | 0.49482904 | −2.0209 |
| SPEC1 | 1.46E−05 | 0.49397108 | −2.02441 |
| KCNH3 | 5.03E−08 | 0.49226165 | −2.03144 |
| LRMP | 3.72E−06 | 0.49226165 | −2.03144 |
| CAMK4 | 5.35E−08 | 0.49141015 | −2.03496 |
| 1110001P04RIK | 2.50E−06 | 0.49141015 | −2.03496 |
| COL15A1 | 7.73E−05 | 0.49055919 | −2.03849 |
| E130307M08RIK | 3.42E−05 | 0.48971117 | −2.04202 |
| NFE2 | 3.74E−05 | 0.48971117 | −2.04202 |
| ASB13 | 4.76E−07 | 0.48717037 | −2.05267 |
| XLR4A | 0.000619052 | 0.48717037 | −2.05267 |
| LOC382020 | 0.0011986 | 0.48717037 | −2.05267 |
| 3110018A08RIK | 0.00353392 | 0.48717037 | −2.05267 |
| BC020108 | 0.000419903 | 0.48632692 | −2.05623 |
| SOX9 | 0.00119698 | 0.48632692 | −2.05623 |
| CD5 | 2.42E−06 | 0.48548638 | −2.05979 |
| ZFP96 | 2.60E−05 | 0.48548638 | −2.05979 |
| AKAP8L | 8.62E−07 | 0.48464405 | −2.06337 |
| 5530400P07RIK | 1.11E−06 | 0.48380464 | −2.06695 |
| A430107D22RIK | 2.94E−06 | 0.48380464 | −2.06695 |
| 0610012D17RIK | 7.81E−08 | 0.48296813 | −2.07053 |
| GALNT2 | 5.48E−07 | 0.48296813 | −2.07053 |
| EPPB9 | 2.51E−05 | 0.48296813 | −2.07053 |
| NSG2 | 8.56E−08 | 0.48213218 | −2.07412 |
| DUSP10 | 4.51E−08 | 0.48129681 | −2.07772 |
| 9430080K19RIK | 3.61E−08 | 0.48129681 | −2.07772 |
| RNASEN | 2.84E−06 | 0.48129681 | −2.07772 |
| GAS6 | 0.000310919 | 0.48129681 | −2.07772 |
| 1810015C11RIK | 7.75E−09 | 0.48046432 | −2.08132 |
| SLITL2 | 0.000449972 | 0.48046432 | −2.08132 |
| LOC386330 | 0.00291956 | 0.48046432 | −2.08132 |
| FKBP9 | 0.000504748 | 0.47963241 | −2.08493 |
| ZFPN1A1 | 8.59E−07 | 0.47880108 | −2.08855 |
| DDX6 | 3.03E−07 | 0.47797263 | −2.09217 |
| BACH1 | 1.88E−06 | 0.47797263 | −2.09217 |
| TNNT1 | 0.000226617 | 0.47797263 | −2.09217 |
| BLK | 2.09E−08 | 0.47714477 | −2.0958 |
| MSCP | 3.61E−07 | 0.47714477 | −2.0958 |
| 2900060B14RIK | 0.0188022 | 0.47714477 | −2.0958 |
| CNN3 | 2.16E−06 | 0.47549535 | −2.10307 |
| REEP1 | 3.13E−08 | 0.47467153 | −2.10672 |
| SDH1 | 4.36E−08 | 0.47302795 | −2.11404 |
| PPARGC1B | 9.21E−07 | 0.47302795 | −2.11404 |
| TLK1 | 5.15E−07 | 0.47302795 | −2.11404 |
| A630097D09RIK | 3.46E−05 | 0.47221042 | −2.1177 |
| 3110078M01RIK | 8.46E−07 | 0.47139126 | −2.12138 |
| 1110015K06RIK | 2.05E−06 | 0.47057716 | −2.12505 |
| EXT1 | 5.14E−06 | 0.46894857 | −2.13243 |
| FBLN2 | 1.48E−07 | 0.4681363 | −2.13613 |
| 1810018P12RIK | 4.02E−05 | 0.46651583 | −2.14355 |
| DCAMKL2 | 1.02E−06 | 0.46570979 | −2.14726 |
| PPT1 | 1.66E−06 | 0.46570979 | −2.14726 |
| 2810036L13RIK | 3.90E−05 | 0.46570979 | −2.14726 |
| H2-EB1 | 0.00495563 | 0.46490221 | −2.15099 |
| 2810470K03RIK | 6.54E−06 | 0.46409742 | −2.15472 |
| RAG1 | 2.91E−05 | 0.46329327 | −2.15846 |
| 2610020H15RIK | 1.83E−07 | 0.46249191 | −2.1622 |
| D10UCLA1 | 4.00E−05 | 0.46169117 | −2.16595 |
| 1110003A17RIK | 7.74E−07 | 0.46089109 | −2.16971 |
| KCTD2 | 2.52E−05 | 0.46089109 | −2.16971 |
| G630024G08RIK | 3.72E−08 | 0.45929709 | −2.17724 |
| 1700026B20RIK | 3.84E−06 | 0.45850107 | −2.18102 |
| FAS | 7.68E−09 | 0.4577078 | −2.1848 |
| 4933424M23RIK | 1.76E−07 | 0.4577078 | −2.1848 |
| 4921518A06RIK | 4.47E−06 | 0.4577078 | −2.1848 |
| IGTP | 0.00151097 | 0.4577078 | −2.1848 |
| 9430068D06RIK | 6.51E−08 | 0.45691518 | −2.18859 |
| A930005H10RIK | 1.37E−07 | 0.45691518 | −2.18859 |
| ABCA3 | 2.04E−07 | 0.45533401 | −2.19619 |
| 5330403D14RIK | 3.86E−07 | 0.45533401 | −2.19619 |
| 4631427C17RIK | 9.36E−07 | 0.45533401 | −2.19619 |
| TRIM28 | 2.42E−05 | 0.45454752 | −2.19999 |
| CERK | 1.15E−05 | 0.45454752 | −2.19999 |
| CRYL1 | 7.94E−07 | 0.45375963 | −2.20381 |
| IL7R | 1.25E−08 | 0.45297446 | −2.20763 |
| IHPK1 | 4.52E−07 | 0.45297446 | −2.20763 |
| RENBP | 1.68E−08 | 0.45218996 | −2.21146 |
| TPST1 | 1.05E−05 | 0.44984458 | −2.22299 |
| 9430029L20RIK | 9.20E−09 | 0.44828776 | −2.23071 |
| 5730593F17RIK | 3.51E−06 | 0.44828776 | −2.23071 |
| C530015C18 | 1.57E−08 | 0.4467377 | −2.23845 |
| HMGN2 | 0.00010418 | 0.4467377 | −2.23845 |
| TRAF4 | 8.39E−08 | 0.44596469 | −2.24233 |
| AXIN2 | 2.23E−09 | 0.44519237 | −2.24622 |
| BCL7A | 5.46E−07 | 0.44519237 | −2.24622 |
| A630082K20RIK | 7.65E−08 | 0.44442074 | −2.25012 |
| TNRC6C | 1.44E−07 | 0.44442074 | −2.25012 |
| PCOLCE | 0.000259954 | 0.44365179 | −2.25402 |
| PRICKLE1 | 6.89E−07 | 0.44288353 | −2.25793 |
| BCL6 | 1.24E−05 | 0.44288353 | −2.25793 |
| COL2A1 | 3.25E−05 | 0.44211792 | −2.26184 |
| MRPL14 | 5.42E−07 | 0.44135106 | −2.26577 |
| ZFP148 | 8.82E−07 | 0.44058686 | −2.2697 |
| CNOT2 | 1.44E−07 | 0.4398253 | −2.27363 |
| C230075L19RIK | 1.84E−07 | 0.43906251 | −2.27758 |
| 2700083E18RIK | 2.52E−07 | 0.43906251 | −2.27758 |
| CCND1 | 1.92E−05 | 0.43830237 | −2.28153 |
| CUTL1 | 8.66E−08 | 0.43754485 | −2.28548 |
| AI467606 | 5.41E−07 | 0.43603003 | −2.29342 |
| GMFG | 7.47E−05 | 0.43603003 | −2.29342 |
| GLDC | 0.000564796 | 0.43603003 | −2.29342 |
| CNP1 | 1.01E−07 | 0.43527466 | −2.2974 |
| RBM38 | 2.02E−08 | 0.4345219 | −2.30138 |
| BC039093 | 2.61E−06 | 0.4345219 | −2.30138 |
| 6.33E+19 | 2.02E−05 | 0.4345219 | −2.30138 |
| SCARA3 | 5.87E−05 | 0.4345219 | −2.30138 |
| FKBP5 | 3.81E−07 | 0.43376985 | −2.30537 |
| BC063749 | 1.34E−09 | 0.43301853 | −2.30937 |
| LOC226135 | 0.000314737 | 0.43301853 | −2.30937 |
| AFF1 | 5.19E−07 | 0.43226794 | −2.31338 |
| COL4A1 | 3.11E−05 | 0.43002799 | −2.32543 |
| COL6A3 | 0.00167802 | 0.43002799 | −2.32543 |
| VAMP4 | 4.17E−07 | 0.4292822 | −2.32947 |
| NUP210 | 0.000100262 | 0.42853898 | −2.33351 |
| ADCY6 | 1.78E−07 | 0.42779834 | −2.33755 |
| UHRF1 | 0.000151815 | 0.42779834 | −2.33755 |
| PTPRS | 3.83E−07 | 0.4270566 | −2.34161 |
| LBH | 5.98E−05 | 0.42631743 | −2.34567 |
| SCML4 | 1.21E−07 | 0.425579 | −2.34974 |
| 1700095N21RIK | 2.00E−07 | 0.425579 | −2.34974 |
| 5930416I19RIK | 4.45E−07 | 0.425579 | −2.34974 |
| SEMA4B | 1.98E−06 | 0.425579 | −2.34974 |
| SCA2 | 6.05E−07 | 0.42484313 | −2.35381 |
| 5830431A10RIK | 2.60E−06 | 0.42484313 | −2.35381 |
| MSH6 | 0.000132082 | 0.42484313 | −2.35381 |
| TTC3 | 5.71E−09 | 0.4219071 | −2.37019 |
| KCTD1 | 1.30E−06 | 0.4219071 | −2.37019 |
| BC028975 | 1.19E−08 | 0.42117677 | −2.3743 |
| GPSM1 | 2.16E−06 | 0.42117677 | −2.3743 |
| ERICH1 | 3.26E−08 | 0.42044896 | −2.37841 |
| GATA3 | 6.03E−07 | 0.42044896 | −2.37841 |
| TTYH3 | 4.09E−06 | 0.42044896 | −2.37841 |
| H2-OB | 6.36E−06 | 0.42044896 | −2.37841 |
| BHLHB9 | 1.72E−05 | 0.42044896 | −2.37841 |
| AW046396 | 0.00133505 | 0.42044896 | −2.37841 |
| 4632417D23 | 5.33E−07 | 0.41754351 | −2.39496 |
| PDLIM1 | 8.32E−07 | 0.41754351 | −2.39496 |
| 1810010N17RIK | 1.37E−06 | 0.41682124 | −2.39911 |
| CHRNA9 | 1.78E−09 | 0.41609973 | −2.40327 |
| GSTM2 | 0.000400316 | 0.41537899 | −2.40744 |
| SCL000121.1_106 | 6.07E−06 | 0.41394155 | −2.4158 |
| MNS1 | 0.000531845 | 0.41394155 | −2.4158 |
| GABABRBP | 3.45E−05 | 0.41250892 | −2.42419 |
| ANP32E | 3.77E−05 | 0.41108279 | −2.4326 |
| SMARCD2 | 6.84E−06 | 0.40402082 | −2.47512 |
| CASP6 | 6.84E−07 | 0.40332013 | −2.47942 |
| LTAP | 3.13E−08 | 0.40053511 | −2.49666 |
| EFEMP2 | 8.18E−05 | 0.39984166 | −2.50099 |
| 4932408F19RIK | 3.03E−07 | 0.39845876 | −2.50967 |
| CD1D1 | 9.54E−07 | 0.39845876 | −2.50967 |
| SH2D1A | 1.27E−05 | 0.39845876 | −2.50967 |
| ADRB2 | 1.53E−08 | 0.39776773 | −2.51403 |
| 6330403E01RIK | 2.81E−07 | 0.39707909 | −2.51839 |
| C230082I21RIK | 1.32E−09 | 0.39639283 | −2.52275 |
| FBXL12 | 3.35E−06 | 0.39502115 | −2.53151 |
| SMO | 3.82E−09 | 0.39433732 | −2.5359 |
| 6720469N11RIK | 1.55E−07 | 0.39365429 | −2.5403 |
| ZFPN1A2 | 1.20E−07 | 0.39297209 | −2.54471 |
| PHF2 | 2.68E−07 | 0.39297209 | −2.54471 |
| LIP1 | 8.34E−07 | 0.39297209 | −2.54471 |
| IFNGR1 | 1.21E−07 | 0.39229224 | −2.54912 |
| SPATA13 | 1.64E−07 | 0.39161321 | −2.55354 |
| NEDD4L | 4.00E−09 | 0.390935 | −2.55797 |
| SLA | 4.00E−05 | 0.390935 | −2.55797 |
| ARHGEF18 | 2.56E−05 | 0.390935 | −2.55797 |
| RASGRP1 | 7.85E−08 | 0.39025761 | −2.56241 |
| NOTCH1 | 2.02E−08 | 0.38823493 | −2.57576 |
| 2900016B01RIK | 6.34E−09 | 0.38756235 | −2.58023 |
| PITPNM2 | 1.79E−07 | 0.3868906 | −2.58471 |
| SMAD3 | 2.00E−06 | 0.38555257 | −2.59368 |
| CHDH | 1.11E−07 | 0.38355324 | −2.6072 |
| C920011N12RIK | 5.34E−06 | 0.38355324 | −2.6072 |
| NISCH | 5.43E−07 | 0.38288944 | −2.61172 |
| 2310007G05RIK | 1.06E−06 | 0.38288944 | −2.61172 |
| SIT1 | 3.76E−09 | 0.38222647 | −2.61625 |
| SLC29A3 | 7.22E−07 | 0.3809045 | −2.62533 |
| AEBP1 | 0.000162054 | 0.3809045 | −2.62533 |
| C730009F21RIK | 9.48E−09 | 0.37958587 | −2.63445 |
| PLEKHG2 | 9.65E−07 | 0.37958587 | −2.63445 |
| MBP | 2.65E−08 | 0.37827348 | −2.64359 |
| D8ERTD325E | 9.64E−06 | 0.37827348 | −2.64359 |
| VPS54 | 4.16E−08 | 0.37761784 | −2.64818 |
| MLL | 2.80E−07 | 0.37761784 | −2.64818 |
| LOC386144 | 4.21E−05 | 0.37761784 | −2.64818 |
| LOC386360 | 0.000403683 | 0.37761784 | −2.64818 |
| BACH2 | 0.000136247 | 0.37696446 | −2.65277 |
| BDH | 2.28E−10 | 0.37500938 | −2.6666 |
| KLHL6 | 2.22E−08 | 0.37500938 | −2.6666 |
| DAP3 | 1.93E−08 | 0.37371163 | −2.67586 |
| TCRB-V8.2 | 8.04E−08 | 0.37371163 | −2.67586 |
| A130062D16RIK | 1.07E−07 | 0.37371163 | −2.67586 |
| SSBP3 | 1.03E−07 | 0.37371163 | −2.67586 |
| MAPK1 | 5.13E−08 | 0.37242006 | −2.68514 |
| FRMD6 | 3.31E−08 | 0.37177485 | −2.6898 |
| TNFRSF13B | 6.85E−08 | 0.37177485 | −2.6898 |
| MMP2 | 0.000109136 | 0.37177485 | −2.6898 |
| ECM1 | 1.84E−07 | 0.3711305 | −2.69447 |
| CUL7 | 2.80E−07 | 0.36602149 | −2.73208 |
| NOTCH3 | 7.70E−06 | 0.36602149 | −2.73208 |
| D930015E06RIK | 2.19E−07 | 0.36538757 | −2.73682 |
| A430106G13RIK | 1.00E−06 | 0.3647545 | −2.74157 |
| 5830468F06RIK | 5.09E−06 | 0.36412363 | −2.74632 |
| HIBADH | 1.23E−08 | 0.36349361 | −2.75108 |
| TMEM9 | 4.73E−07 | 0.36223616 | −2.76063 |
| TSPAN32 | 8.74E−07 | 0.35662957 | −2.80403 |
| H2-T9 | 4.22E−05 | 0.35662957 | −2.80403 |
| ESM1 | 0.00132143 | 0.35601252 | −2.80889 |
| ALOX5AP | 1.28E−07 | 0.35539634 | −2.81376 |
| RFX2 | 1.13E−06 | 0.35539634 | −2.81376 |
| 2610019F03RIK | 3.33E−06 | 0.35478103 | −2.81864 |
| WHRN | 4.89E−07 | 0.35355303 | −2.82843 |
| 5830496L11RIK | 1.90E−07 | 0.35294159 | −2.83333 |
| GSTP1 | 7.09E−07 | 0.35294159 | −2.83333 |
| 3100002J23RIK | 3.91E−07 | 0.35232978 | −2.83825 |
| YPEL3 | 0.0001326 | 0.35111127 | −2.8481 |
| A130092J06RIK | 3.76E−06 | 0.35050332 | −2.85304 |
| IGSF3 | 5.35E−07 | 0.34929007 | −2.86295 |
| HDAC7A | 1.07E−06 | 0.34448192 | −2.90291 |
| IDB3 | 1.63E−07 | 0.34388605 | −2.90794 |
| OLFML3 | 6.69E−07 | 0.34328988 | −2.91299 |
| 6430510M02RIK | 1.07E−07 | 0.34269578 | −2.91804 |
| TRBV13- | 1.50E−05 | 0.34151023 | −2.92817 |
| 1_M15618_T_CELL_RECEPTOR_BETA_VARIABLE_13- | | | |
| CD97 | 2.26E−05 | 0.33973969 | −2.94343 |
| MTF2 | 1.12E−06 | 0.33797714 | −2.95878 |
| PLA2G12A | 1.72E−08 | 0.33622487 | −2.9742 |
| D15WSU75E | 2.04E−06 | 0.33622487 | −2.9742 |
| ETHE1 | 1.66E−08 | 0.33448172 | −2.9897 |
| HIVEP3 | 3.41E−09 | 0.33390319 | −2.99488 |
| CYB5 | 3.77E−08 | 0.33390319 | −2.99488 |
| CTSE | 0.149884 | 0.33332444 | −3.00008 |
| ZFP219 | 2.26E−07 | 0.32987732 | −3.03143 |
| ABHD8 | 1.69E−06 | 0.32987732 | −3.03143 |
| 4732481H14RIK | 8.63E−07 | 0.32930592 | −3.03669 |
| PRNP | 3.14E−05 | 0.32873542 | −3.04196 |
| A630038E17RIK | 0.000216397 | 0.32873542 | −3.04196 |
| A930013B10RIK | 8.09E−08 | 0.3253355 | −3.07375 |
| ETS1 | 5.90E−07 | 0.32252862 | −3.1005 |
| LOC385086 | 0.00104356 | 0.32252862 | −3.1005 |
| RNPEPL1 | 7.60E−09 | 0.32196994 | −3.10588 |
| KLF13 | 6.51E−07 | 0.32141319 | −3.11126 |
| KCNN4 | 1.98E−06 | 0.3208563 | −3.11666 |
| NIPSNAP1 | 1.39E−06 | 0.3208563 | −3.11666 |
| C920004C08RIK | 0.00056251 | 0.3208563 | −3.11666 |
| TRBV7_AE000663_T_CELL_RECEPTOR_BETA_VARIABLE_7_29 | 6.95E−05 | 0.31974625 | −3.12748 |
| SLC43A1 | 3.64E−08 | 0.31643867 | −3.16017 |
| STK4 | 7.89E−07 | 0.31589089 | −3.16565 |
| WISP2 | 1.27E−05 | 0.31262407 | −3.19873 |
| ACTN2 | 3.36E−07 | 0.31046452 | −3.22098 |
| EPB4.1L4B | 4.42E−07 | 0.31046452 | −3.22098 |
| RNF144 | 2.74E−09 | 0.30885455 | −3.23777 |
| SCL0001849.1_2273 | 1.73E−05 | 0.30778606 | −3.24901 |
| ART4 | 6.83E−07 | 0.30619056 | −3.26594 |
| 18S_RRNA_X00686_301 | 0.00445027 | 0.29988874 | −3.33457 |
| A330103N21RIK | 1.65E−06 | 0.29936983 | −3.34035 |
| TPCN1 | 2.60E−09 | 0.29885092 | −3.34615 |
| AJ237586 | 1.97E−07 | 0.29833381 | −3.35195 |
| BC026370 | 6.26E−09 | 0.29575998 | −3.38112 |
| EPHX1 | 4.14E−09 | 0.29524827 | −3.38698 |
| COL5A1 | 0.000124246 | 0.29371767 | −3.40463 |
| SNN | 1.47E−08 | 0.29270149 | −3.41645 |
| BCL9L | 1.00E−08 | 0.29219432 | −3.42238 |
| 0710008K08RIK | 9.65E−08 | 0.29168806 | −3.42832 |
| F730003H07RIK | 0.000110195 | 0.28967371 | −3.45216 |
| AI504432 | 2.49E−08 | 0.28717477 | −3.4822 |
| OACT1 | 9.74E−09 | 0.28667752 | −3.48824 |
| A130093I21RIK | 1.16E−06 | 0.28519116 | −3.50642 |
| RGS10 | 2.26E−05 | 0.28469751 | −3.5125 |
| 1110046J11RIK | 3.46E−06 | 0.28420394 | −3.5186 |
| E2F2 | 2.29E−06 | 0.27835537 | −3.59253 |
| BRD3 | 3.58E−07 | 0.27787349 | −3.59876 |
| AA408556 | 4.12E−06 | 0.27262665 | −3.66802 |
| SATB1 | 1.49E−07 | 0.27074371 | −3.69353 |
| ILVBL | 3.59E−07 | 0.27074371 | −3.69353 |
| TIAM1 | 1.97E−09 | 0.26887358 | −3.71922 |
| POU6F1 | 2.59E−08 | 0.26887358 | −3.71922 |
| MAGED1 | 1.17E−05 | 0.26887358 | −3.71922 |
| 1810055G02RIK | 1.69E−09 | 0.26655436 | −3.75158 |
| 0710001E13RIK | 6.72E−10 | 0.26471273 | −3.77768 |
| LMAN2L | 4.14E−08 | 0.26471273 | −3.77768 |
| SLC29A1 | 1.20E−07 | 0.26425455 | −3.78423 |
| 3830612M24 | 2.98E−07 | 0.26379726 | −3.79079 |
| H2-T10 | 0.000216723 | 0.26379726 | −3.79079 |
| CXCR4 | 4.41E−08 | 0.26334015 | −3.79737 |
| RIL-PENDING | 2.35E−08 | 0.26242931 | −3.81055 |
| SERPINH1 | 3.41E−05 | 0.26242931 | −3.81055 |
| PALM | 2.59E−09 | 0.26197487 | −3.81716 |
| CXCL12 | 0.000158114 | 0.26106866 | −3.83041 |
| 5430417L22RIK | 1.68E−08 | 0.2606162 | −3.83706 |
| TRIB2 | 4.82E−08 | 0.26016531 | −3.84371 |
| ETS2 | 5.78E−08 | 0.26016531 | −3.84371 |
| ALDH2 | 9.48E−07 | 0.25881592 | −3.86375 |
| HMGN1 | 3.20E−05 | 0.25702844 | −3.89062 |
| TRP53INP1 | 4.08E−08 | 0.25569562 | −3.9109 |
| ITPR2 | 5.70E−08 | 0.25392956 | −3.9381 |
| TCRB | 8.36E−06 | 0.25348992 | −3.94493 |
| A930023F05RIK | 4.58E−09 | 0.24784315 | −4.03481 |
| SLC5A9 | 1.20E−06 | 0.24698554 | −4.04882 |
| ICAM2 | 2.53E−07 | 0.24655805 | −4.05584 |
| H2-DMA | 1.25E−05 | 0.24655805 | −4.05584 |
| 4932414K18RIK | 3.75E−08 | 0.24443125 | −4.09113 |
| TAP2 | 9.85E−09 | 0.24358518 | −4.10534 |
| TRBV12- | 3.25E−05 | 0.24358518 | −4.10534 |
| 2_M15613_T_CELL_RECEPTOR_BETA_VARIABLE_12- | | | |
| PRELP | 8.99E−06 | 0.24232261 | −4.12673 |
| TRBV12- | 1.47E−06 | 0.24106609 | −4.14824 |
| 1_M15614_T_CELL_RECEPTOR_BETA_VARIABLE_12- | | | |
| LOX | 8.44E−06 | 0.24023216 | −4.16264 |
| 1500004A08RIK | 1.03E−07 | 0.2398162 | −4.16986 |
| 6720418B01RIK | 4.27E−08 | 0.23857238 | −4.1916 |
| 4930572J05RIK | 4.75E−07 | 0.23692472 | −4.22075 |
| SCL0001032.1_178 | 7.57E−06 | 0.23692472 | −4.22075 |
| PSAP | 4.23E−08 | 0.23610521 | −4.2354 |
| ASS1 | 5.82E−08 | 0.23569619 | −4.24275 |
| PARD6G | 1.30E−08 | 0.23528803 | −4.25011 |
| 1500009L16RIK | 9.73E−07 | 0.2324511 | −4.30198 |
| GM2A | 2.89E−06 | 0.23124595 | −4.3244 |
| LAT | 5.46E−08 | 0.23084559 | −4.3319 |
| C3 | 9.90E−06 | 0.23084559 | −4.3319 |
| PPAP2B | 1.69E−05 | 0.23044607 | −4.33941 |
| CTLA4 | 5.06E−06 | 0.23044607 | −4.33941 |
| FBP1 | 5.98E−05 | 0.22845811 | −4.37717 |
| B3BP | 1.28E−10 | 0.22453292 | −4.45369 |
| PRKCB | 6.59E−08 | 0.22453292 | −4.45369 |
| PPP1R1C | 3.85E−09 | 0.22067553 | −4.53154 |
| RAPGEF3 | 6.21E−10 | 0.21953174 | −4.55515 |
| BAMBI-PS1 | 7.87E−08 | 0.21915118 | −4.56306 |
| 1700012H17RIK | 5.26E−09 | 0.21839306 | −4.5789 |
| ACVR2B | 3.12E−10 | 0.21801502 | −4.58684 |
| 18S_RRNA_X00686_849 | 0.000602805 | 0.21538634 | −4.64282 |
| SERPINF1 | 1.43E−05 | 0.21464156 | −4.65893 |
| NAV1 | 4.40E−08 | 0.21426995 | −4.66701 |
| TBXA2R | 3.11E−08 | 0.2135283 | −4.68322 |
| SCL0001132.1_96 | 1.63E−05 | 0.20732994 | −4.82323 |
| SOX4 | 1.93E−09 | 0.2055414 | −4.8652 |
| E430021E22RIK | 3.57E−07 | 0.20447556 | −4.89056 |
| LOC381739 | 2.02E−08 | 0.203063 | −4.92458 |
| CD6 | 3.02E−10 | 0.20096261 | −4.97605 |
| H2-OA | 1.09E−07 | 0.19888426 | −5.02805 |
| LOC384370 | 7.41E−06 | 0.1978529 | −5.05426 |
| ZDHHC8 | 3.73E−09 | 0.19751019 | −5.06303 |
| AI481316 | 1.75E−09 | 0.19682715 | −5.0806 |
| H2-BL | 3.80E−06 | 0.19614612 | −5.09824 |
| AA407270 | 7.17E−09 | 0.1954675 | −5.11594 |
| ITGAE | 6.39E−06 | 0.1951288 | −5.12482 |
| GPR83 | 1.37E−07 | 0.19479128 | −5.1337 |
| SBK | 4.54E−10 | 0.18783011 | −5.32396 |
| RPS6KL1 | 1.75E−09 | 0.18750504 | −5.33319 |
| TCF7 | 8.08E−08 | 0.18396455 | −5.43583 |
| NRP | 4.24E−08 | 0.18364596 | −5.44526 |
| DNAJC6 | 3.58E−11 | 0.18269378 | −5.47364 |
| SCL0001090.1_202 | 3.66E−09 | 0.18269378 | −5.47364 |
| SCL0001131.1_227 | 1.31E−06 | 0.17924456 | −5.57897 |
| IL17RB | 1.94E−09 | 0.17800626 | −5.61778 |
| ACAS2L | 2.63E−07 | 0.17647048 | −5.66667 |
| AKR1C12 | 8.40E−09 | 0.1761652 | −5.67649 |
| COL6A1 | 3.56E−05 | 0.17464534 | −5.72589 |
| SOCS3 | 3.74E−08 | 0.17283904 | −5.78573 |
| LDH2 | 1.01E−07 | 0.17283904 | −5.78573 |
| DGKA | 6.54E−07 | 0.17283904 | −5.78573 |
| GM525 | 1.28E−07 | 0.17253963 | −5.79577 |
| TIMP2 | 2.00E−08 | 0.17224096 | −5.80582 |
| AQP11 | 3.40E−09 | 0.17045939 | −5.8665 |
| TRBV6_AE000663_T_CELL_RECEPTOR_BETA_VARIABLE_6_11 | 5.66E−08 | 0.16695132 | −5.98977 |
| TNFRSF7 | 9.20E−08 | 0.1615441 | −6.19026 |
| CD2 | 7.22E−08 | 0.15932015 | −6.27667 |
| DTX1 | 3.94E−09 | 0.15876897 | −6.29846 |
| AI875142 | 2.49E−07 | 0.15712672 | −6.36429 |
| IGFBP4 | 1.57E−08 | 0.15577125 | −6.41967 |
| SH3KBP1 | 2.13E−10 | 0.15496356 | −6.45313 |
| 2510015F01RIK | 3.11E−06 | 0.15362659 | −6.50929 |
| TRBV11_AE000663_T_CELL_RECEPTOR_BETA_VARIABLE_11_{—} | 2.50E−08 | 0.15151148 | −6.60016 |
| SLC16A5 | 1.12E−10 | 0.14865092 | −6.72717 |
| TUBB2B | 4.82E−08 | 0.14813652 | −6.75053 |
| DNTT | 7.39E−07 | 0.14533954 | −6.88044 |
| SYTL1 | 6.70E−08 | 0.14309058 | −6.98858 |
| 2410008J05RIK | 2.59E−09 | 0.13726158 | −7.28536 |
| 0610041G09RIK | 9.40E−06 | 0.13702403 | −7.29799 |
| 2210408F11RIK | 2.70E−08 | 0.13513751 | −7.39987 |
| LOC386545 | 2.77E−05 | 0.13121465 | −7.6211 |
| TRBV31_X03277_T_CELL_RECEPTOR_BETA_VARIABLE_31_33 | 4.97E−06 | 0.12918411 | −7.74089 |
| TCRB-V8.3 | 1.15E−08 | 0.12829181 | −7.79473 |
| KLF2 | 3.06E−06 | 0.125434 | −7.97232 |
| TCRB-V13 | 5.74E−08 | 0.12435182 | −8.0417 |
| A130038J17RIK | 8.77E−09 | 0.11784809 | −8.4855 |
| LOC381738 | 1.87E−09 | 0.11723701 | −8.52973 |
| G22P1 | 5.44E−10 | 0.11582345 | −8.63383 |
| CD27 | 4.55E−08 | 0.11383376 | −8.78474 |
| TMEM108 | 8.61E−08 | 0.11265625 | −8.87656 |
| ACTN1 | 2.20E−09 | 0.11091295 | −9.01608 |
| ST6GAL1 | 6.69E−10 | 0.10657936 | −9.38268 |
| 9626100_15 | 1.11E−05 | 0.10348549 | −9.66319 |
| 9626100_224 | 8.13E−06 | 0.10118027 | −9.88335 |
| C030046M14RIK | 1.45E−12 | 0.1010051 | −9.90049 |
| SELL | 6.85E−09 | 0.09944213 | −10.0561 |
| COX6A2 | 7.37E−05 | 0.0987547 | −10.1261 |
| FRAT2 | 6.49E−09 | 0.09841357 | −10.1612 |
| LY6D | 1.48E−09 | 0.09790292 | −10.2142 |
| 9130430L19RIK | 2.71E−10 | 0.09278245 | −10.7779 |
| CDCA7 | 3.37E−07 | 0.09214212 | −10.8528 |
| LOC382896 | 3.09E−09 | 0.09150554 | −10.9283 |
| CD8B | 7.37E−08 | 0.08993372 | −11.1193 |
| E430002D04RIK | 2.09E−10 | 0.08931203 | −11.1967 |
| TRGV2_M12831_T_CELL_RECEPTOR_GAMMA_VARIABLE_2_3 | 2.57E−11 | 0.08656959 | −11.5514 |
| PP11R | 2.12E−07 | 0.08641922 | −11.5715 |
| CD81 | 4.58E−08 | 0.08318706 | −12.0211 |
| IGH-6 | 8.08E−09 | 0.08289881 | −12.0629 |
| AI132321 | 2.09E−08 | 0.08204186 | −12.1889 |
| 9626958_317 | 8.03E−06 | 0.07721352 | −12.9511 |
| TRBV8_AE000663_T_CELL_RECEPTOR_BETA_VARIABLE_8_27 | 9.31E−08 | 0.07694675 | −12.996 |
| MGST2 | 3.44E−09 | 0.07419664 | −13.4777 |
| RAMP1 | 5.23E−08 | 0.072043 | −13.8806 |
| NCK2 | 3.02E−08 | 0.06863088 | −14.5707 |
| MARCKS | 1.18E−09 | 0.06572116 | −15.2158 |
| DPP4 | 2.19E−10 | 0.05642577 | −17.7224 |
| TRBV1_AE000663_T_CELL_RECEPTOR_BETA_VARIABLE_1_20 | 6.96E−07 | 0.05603622 | −17.8456 |
| H19 | 3.39E−07 | 0.0557454 | −17.9387 |
| TCRG-V4 | 3.97E−09 | 0.05129626 | −19.4946 |
| 1190002H23RIK | 4.58E−10 | 0.05024065 | −19.9042 |
| BCL11B | 2.77E−10 | 0.0495491 | −20.182 |
| CD3G | 8.20E−11 | 0.04647186 | −21.5184 |
| BGN | 3.71E−07 | 0.03969215 | −25.1939 |
| CD3D | 1.53E−09 | 0.0349758 | −28.5912 |
| CD3E | 7.19E−10 | 0.03190668 | −31.3414 |
| LOC434197 | 5.40E−11 | 0.02356029 | −42.4443 |
| MYLC2PL | 8.10E−10 | 0.01703917 | −58.6883 |
| PDLIM4 | 8.76E−11 | 0.00978646 | −102.182 |

TABLE 2
Comparison of cell surface receptor repertoires of ITNKs and LAKs.

| Cell Type | Ly49C/I | Ly49D | Ly49G2 | NK1.1 | NKp46 | NKG2A/C/E | NKG2D | CD3 |
|---|---|---|---|---|---|---|---|---|
| DN3- | − | − | − | + | + | + | − | − |
| reprogrammed | | | | | | | | |
| ITNK (in vitro) | | | | | | | | |
| DP- | − | − | − | + | + | + | ND | + |
| reprogrammed | | | | | | | | |
| ITNK (in vitro) | | | | | | | | |
| DP- | + | − | + | + | + | + | + | low |
| reprogrammed | | | | | | | | |
| ITNK (in vivo) | | | | | | | | |
| LAK | + | + | + | + | + | + | + | − |

Note:
N.D., not determined. +, present;. −, absent; low, low levels.

TABLE 3

| | |
|---|---|
| | Fold-Change |
| Column ID | (+OHT vs. −OHT) |

24 hours +OHT vs. −OHT

| | |
|---|---|
| TCRB-V13 | −14894.3 |
| TCRB-V13 | −412.694 |
| HIST1H2AO | −35.3085 |
| MTDNA_CYTB | −13.7646 |
| IFITM1 | −12.6519 |
| CDCA7 | −12.2615 |
| PDLIM4 | −10.206 |
| CD3D | −9.81142 |
| RPS29 | −9.27924 |
| MTDNA_COXIII | −8.38116 |
| RPS14 | −6.99771 |
| MYLC2PL | −6.76599 |
| CD3D | −5.74896 |
| IFITM2 | −5.49522 |
| RPL13 | −5.33629 |
| RPS17 | −5.30823 |
| RPL41 | −5.29871 |
| 18S_RRNA_X00686_301 | −5.08974 |
| HIST2H2AC | −4.87534 |
| IFITM3 | −4.86541 |
| MTDNA_ND4 | −4.61459 |
| HIST1H2AI | −4.60413 |
| MT-CYTB | −4.34 |
| MYLC2PL | −4.32543 |
| RPS11 | −4.20813 |
| ITGB7 | −4.05052 |
| RPL39 | −3.69839 |
| RPS27L | −3.63035 |
| CD3G | −3.49152 |
| EG668668 | −3.46858 |
| CD160 | −3.44386 |
| RPL23 | −3.38327 |
| CD3E | −3.30996 |
| HIST1H2AO | −3.29719 |
| EMP3 | −3.22525 |
| PDLIM4 | −3.15574 |
| TBCA | −3.1281 |
| THY1 | −3.11285 |
| CD8B | −3.01933 |
| PRKACB | −2.90749 |
| HIST1H2AF | −2.78505 |
| LOC226574 | −2.74888 |
| G22P1 | −2.74634 |
| HIST1H2AG | −2.64205 |
| AI481316 | −2.62333 |
| IGH-6 | −2.5636 |
| A130092J06RIK | −2.55682 |
| TCRG-V4 | −2.54552 |
| UBB | −2.45105 |
| LOC434197 | −2.4265 |
| RPS17 | −2.41723 |
| MTDNA_ATP6 | −2.41506 |
| TXNIP | −2.40294 |
| LOC381808 | −2.39227 |
| PPIA | −2.37637 |
| LOC382896 | −2.35265 |
| HMGCS1 | −2.30359 |
| TCRB-V8.2 | −2.2788 |
| HMGN2 | −2.2528 |
| UPP1 | −2.22963 |
| CSTB | −2.21395 |
| PSAP | −2.20351 |
| TRBV1_AE000663_T_CELL_RECEPTOR_BETA_VARIABLE_1_207 | −2.17733 |
| HIBADH | −2.16873 |
| E430002D04RIK | −2.15383 |
| CDCA7 | −2.15035 |
| RPS27 | −2.14844 |
| RPL8 | −2.11346 |
| RNPEPL1 | −2.11344 |
| COX6A2 | −2.10037 |
| CD27 | −2.07584 |
| MARCKS | −2.05494 |
| VIM | −2.04688 |
| AA408556 | −2.03945 |
| 4932414K18RIK | −2.01727 |
| BCL11B | −2.011 |
| RPS14 | −2.0086 |
| TCRB-V8.2 | −2.00629 |
| COX7C | 2.11831 |
| LOC270037 | 2.16383 |
| RPA1 | 2.26914 |
| LAPTM5 | 2.8707 |
| UBL5 | 3.35851 |
| ATP5G3 | 3.54832 |
| 1300002F13RIK | 3.6466 |
| CD52 | 3.81745 |
| LDH1 | 3.82439 |
| CYBA | 6.431 |
| FCER1G | 7.97969 |
| HMGCS1 | 27.1719 |

48 hours +OHT vs. −OHT

| | |
|---|---|
| ROG | 11.7941537 |
| FCER1G | 11.6317801 |
| UPP1 | 9.00046788 |
| IFITM1 | 8.6938789 |
| SCIN | 8.6938789 |
| SERPINA3G | 8.51496146 |
| XCL1 | 7.62110398 |
| AQP9 | 7.4127045 |
| NKG7 | 7.01284577 |
| IFITM2 | 6.40855902 |
| IFITM3 | 6.36429187 |
| 9130404D14RIK | 5.69620078 |
| GADD45G | 5.6177795 |
| LGALS3 | 5.46416103 |
| CD160 | 5.31474326 |
| KLRD1 | 5.27803164 |
| VIM | 4.9933222 |
| TYROBP | 4.89056111 |
| LITAF | 4.82323131 |
| BC025206 | 4.78991482 |
| AVIL | 4.72397065 |
| LMNA | 4.72397065 |
| GLRX1 | 4.40762046 |
| NFIL3 | 4.40762046 |
| LTA | 4.1410597 |
| CCR5 | 4.0278222 |
| WBSCR5 | 4 |
| P2RY14 | 3.91768119 |
| 1300002F13RIK | 3.83705648 |
| AMICA1 | 3.73213197 |
| LOC270152 | 3.70635225 |
| 9130211I03RIK | 3.6553258 |
| CDKN2B | 3.6553258 |
| PLCG2 | 3.55537072 |
| CTSW | 3.53081199 |
| BC049975 | 3.50642289 |
| LOC381140 | 3.36358566 |
| LGALS1 | 3.34035168 |
| MT1 | 3.27160823 |
| SYTL2 | 3.27160823 |
| GPR114 | 3.24900959 |
| S100A1 | 3.24900959 |
| 2310067E08RIK | 3.20427951 |
| LRRK1 | 3.20427951 |
| TNFRSF11B | 3.18214594 |
| IDB2 | 3.16016525 |
| CCL4 | 3.11665832 |
| E030006K04RIK | 3.11665832 |
| OSBPL3 | 3.11665832 |
| LY6A | 3.09512999 |
| TNFRSF9 | 3.09512999 |
| S100A6 | 3.07375036 |
| 1500031H04RIK | 3.05251842 |
| 2210411K11RIK | 3.05251842 |
| CTNNA1 | 3.03143313 |
| LOC381319 | 3.03143313 |
| EMILIN2 | 3.01049349 |
| 1110018K11RIK | 2.9896985 |
| ANXA2 | 2.9896985 |
| SIAT10 | 2.96904714 |
| 2310046K01RIK | 2.94853843 |
| CISH | 2.92817139 |
| 1110004P15RIK | 2.90794503 |
| GOLPH2 | 2.88785839 |
| HAVCR2 | 2.88785839 |
| PLSCR1 | 2.88785839 |
| SLC2A6 | 2.8679105 |
| CAPG | 2.84810039 |
| LAG3 | 2.84810039 |
| F2R | 2.82842712 |
| LOC269941 | 2.82842712 |
| 1190002C06RIK | 2.80888975 |
| CD9 | 2.78948733 |
| S100A11 | 2.78948733 |
| GCNT1 | 2.75108364 |
| CDKN1A | 2.73208051 |
| KLRE1 | 2.73208051 |
| GPC1 | 2.71320865 |
| SERPINE2 | 2.69446715 |
| LRP12 | 2.67585511 |
| MLKL | 2.67585511 |
| BC024955 | 2.65737163 |
| BHLHB2 | 2.65737163 |
| C330008K14RIK | 2.65737163 |
| F2RL2 | 2.63901582 |
| GLRX | 2.63901582 |
| IFNG | 2.62078681 |
| PGLYRP1 | 2.62078681 |
| 1110007C02RIK | 2.60268371 |
| BC029169 | 2.60268371 |
| TRAF1 | 2.60268371 |
| CDKN2A | 2.58470566 |
| DUSP6 | 2.58470566 |
| LY6G5B | 2.58470566 |
| RGS1 | 2.5668518 |
| MYO1F | 2.54912125 |
| HBA-A1 | 2.53151319 |
| 2310047C17RIK | 2.51402675 |
| AIM1L | 2.51402675 |
| PILRB | 2.4966611 |
| 2410008K03RIK | 2.4794154 |
| APOB48R | 2.4794154 |
| PDGFA | 2.4794154 |
| FURIN | 2.46228883 |
| SPP1 | 2.46228883 |
| ROM1 | 2.44528056 |
| SH3BP2 | 2.44528056 |
| PPP3CC | 2.42838977 |
| B4GALNT4 | 2.41161566 |
| IER3 | 2.41161566 |
| OSM | 2.41161566 |
| DAPK2 | 2.39495741 |
| LOC218482 | 2.39495741 |
| MAPKAPK3 | 2.39495741 |
| PLP2 | 2.37841423 |
| BAG3 | 2.36198532 |
| OSTF1 | 2.36198532 |
| SERPINB6A | 2.3456699 |
| FXYD4 | 2.32946717 |
| LOC327957 | 2.32946717 |
| AHNAK | 2.29739671 |
| CD69 | 2.28152743 |
| HK2 | 2.28152743 |
| FES | 2.26576777 |
| IL18R1 | 2.26576777 |
| PPAP2C | 2.26576777 |
| SLC39A4 | 2.25011697 |
| TES | 2.25011697 |
| TNF | 2.25011697 |
| HGFAC | 2.23457428 |
| CD244 | 2.21913894 |
| 6330414G02RIK | 2.20381023 |
| CD63 | 2.20381023 |
| LOC383981 | 2.1885874 |
| NAPSA | 2.1885874 |
| PKP3 | 2.1885874 |
| EMP1 | 2.17346973 |
| FOSL2 | 2.17346973 |
| GLIPR1 | 2.17346973 |
| NT5E | 2.17346973 |
| SLC24A3 | 2.17346973 |
| 2610009E16RIK | 2.15845647 |
| 1110020C13RIK | 2.14354693 |
| D10BWG1379E | 2.14354693 |
| ID2 | 2.14354693 |
| DOK2 | 2.12874036 |
| LOC381924 | 2.12874036 |
| 2210008N01RIK | 2.11403608 |
| 5330403J18RIK | 2.11403608 |
| HIST1H1C | 2.09943337 |
| 0610037M15RIK | 2.08493152 |
| 7420404O03RIK | 2.08493152 |
| A430006M23RIK | 2.07052985 |
| D930046M13RIK | 2.07052985 |
| GNG2 | 2.07052985 |
| GPR68 | 2.07052985 |
| H2-Q8 | 2.07052985 |
| IFI30 | 2.07052985 |
| ZFP608 | 2.07052985 |
| DCI | 2.05622765 |
| NFKB1 | 2.05622765 |
| PIM3 | 2.05622765 |
| SGK | 2.05622765 |
| CCNG1 | 2.04202425 |
| CYP51 | 2.04202425 |
| LOC385953 | 2.04202425 |
| EGR1 | 2.02791896 |
| HHEX | 2.02791896 |
| MYO1E | 2.02791896 |
| TMEM126A | 2.02791896 |
| NCF4 | 2.0139111 |
| PDLIM7 | 2.0139111 |
| CXCL9 | 2 |
| GPR18 | 2 |
| MVP | 2 |
| PRSS19 | 2 |
| A130038J17RIK | −2.0139111 |
| A130093I21RIK | −2.0139111 |
| EPHX1 | −2.0139111 |
| NOTCH3 | −2.0139111 |
| MTF2 | −2.027919 |
| TNFRSF7 | −2.027919 |
| 4932414K18RIK | −2.0420243 |
| GFI1 | −2.0420243 |
| 2410008J05RIK | −2.0562277 |
| 2610019F03RIK | −2.0705298 |
| H2-OB | −2.0705298 |
| SATB1 | −2.0705298 |
| TCF7 | −2.0705298 |
| 2900060B14RIK | −2.0849315 |
| TBXA2R | −2.0849315 |
| NISCH | −2.0994334 |
| LOC434197 | −2.1140361 |
| PARD6G | −2.1140361 |
| DPP4 | −2.1435469 |
| H2-AB1 | −2.1435469 |
| LMAN2L | −2.1435469 |
| BRD3 | −2.1584565 |
| CD27 | −2.1584565 |
| LOC386192 | −2.1584565 |
| H2-EB1 | −2.1734697 |
| NCK2 | −2.1734697 |
| RAMP1 | −2.1734697 |
| 1110046J11RIK | −2.1885874 |
| AQP11 | −2.2345743 |
| SLA | −2.2345743 |
| MARCKS | −2.250117 |
| IGH-6 | −2.2657678 |
| SH2D1A | −2.2657678 |
| F730003H07RIK | −2.2973967 |
| H2-T10 | −2.2973967 |
| DGKA | −2.3133764 |
| DNTT | −2.3133764 |
| ETS1 | −2.3294672 |
| LOC268393 | −2.3294672 |
| LOC386360 | −2.3294672 |
| TMEM108 | −2.3294672 |
| C230098O21RIK | −2.3619853 |
| RNPEPL1 | −2.3619853 |
| G22P1 | −2.3784142 |
| TRBV31_X03277_T_CELL_RECEPTOR_BETA_VARIABLE_31_33 | −2.3784142 |
| ALDH2 | −2.4283898 |
| CDCA7 | −2.4622888 |
| NRP | −2.4622888 |
| TXNIP | −2.4622888 |
| SLC16A5 | −2.4966611 |
| ACAS2L | −2.5140267 |
| FRAT2 | −2.5491213 |
| CD81 | −2.6390158 |
| PRKCB | −2.6573716 |
| PDLIM4 | −2.6758551 |
| H2-BL | −2.7132087 |
| PP11R | −2.7320805 |
| ACTN1 | −2.7510836 |
| CD6 | −2.7510836 |
| CD2 | −2.7894873 |
| ST6GAL1 | −2.8088898 |
| TRBV1_AE000663_T_CELL_RECEPTOR_BETA_VARIABLE_1_20 | −2.8088898 |
| CD8B | −2.8481004 |
| 9430068D06RIK | −2.8679105 |
| AI132321 | −3.0737504 |
| H19 | −3.1601652 |
| LY6D | −3.3869812 |
| CTSE | −3.5064229 |
| BCL11B | −3.5801003 |
| LOC382896 | −4.5947934 |
| COX6A2 | −6.2766728 |

TABLE 4
The list of primers in this study.

| | | | |
|---|---|---|---|
| Genotyping PCR | | | Size of PCR |
| primers | Primer sequences (5′-3′) | SEQ ID NO. | products (bp) |

Genotyping primers.

| Bcl11b-cko-FW | TGAGTCAATAAACCTGGGCGAC | 1 | 243 (wild type); |
|---|---|---|---|
| Bcl11b-cko-RV | GGAATCCTTGGAGTCACTTGTGC | 2 | 345 (flox); |
| Bcl11b-cko-DEL | TCCTGGTAACACACAATTGC | 3 | 450 (del) |
| qPCR primers | Primer sequences (5′-3′) | SEQ ID NO. | |

qRT-PCR primers.

| Notch1-Fwd | CCCTTGCTCTGCCTAACGC | 4 |
|---|---|---|
| Notch1-Rev | GGAGTCCTGGCATCGTTGG | 5 |
| Etsi-Fwd | TTAGGAAAGGCTCGTTTGCTC | 6 |
| Ets1-Rev | CCAAAGCACAAGCATAGTTTGC | 7 |
| Hes1-Fwd | CCAGCCAGTGTCAACACGA | 8 |
| Hes1-Rev | AATGCCGGGAGCTATCTTTCT | 9 |
| Gata3-Fwd | CTCGGCCATTCGTACATGGAA | 10 |
| Gata3-Rev | GGATACCTCTGCACCGTAGC | 11 |
| Deltax1-Fwd | TGTTCAGGCTATACACGCATCAA | 12 |
| Deltax1-Rev | CCACCGCCCACTTTCAAG | 13 |
| Tcf1-Fwd | ATGGGCGGCAACTCTTTGAT | 14 |
| Tcf1-Rev | CGTAGCCGGGCTGATTCAT | 15 |
| Cdkn1c-Fwd | CGAGGAGCAGGACGAGAATC | 16 |
| Cdkn1c-Rev | GAAGAAGTCGTTCGCATTGGC | 17 |
| Id2-Fwd | ATGAAAGCCTTCAGTCCGGTG | 18 |
| Id2-Rev | AGCAGACTCATCGGGTCGT | 19 |
| Il2rb-Fwd | TGGAGCCTGTCCCTCTACG | 20 |
| Il2rb-Rev | TCCACATGCAAGAGACATTGG | 21 |
| Zfp105-Fwd | GGCATCCAGCCAACAAGTGTA | 22 |
| Zfp105-Rev | CATTTCCTGACCCTTTTCCTCAT | 23 |
| Traf1-Fwd | GGAGGCATCCTTTGATGGT A | 24 |
| Traf1-Rev | AGGGACAGGTGGGTCTTCTT | 25 |
| Zbtb32-Fwd | GCTCTGAGAGAGGACTTGGGA | 26 |
| Zbtb32-Rev | TGCTTTATGCTTGTGTGACATCT | 27 |
| PCR primers | Primer sequences (5′-3′) | SEQ ID NO. |

Tcrb rearrangement PCR primers.

| | | |
|---|---|---|
| TCRB_Dβ2-Fwd | GTAGGCACCTGTGGGGAAGAAACT | 28 |
| TCRB_Vβ2-Fwd | GGGTCACTGATACGGAGCTG | 29 |
| TCRB_Jβ2-Rev | TGAGAGCTGTCTCCTACTATCGATT | 30 |

List of primers for ChIP assay qPCR.

| | | |
|---|---|---|
| BS1-Fwd | CCGCTACGAGGCACCCTCCTTT | 31 |
| BS1-Rev | AGTCTCCTTGGGAAGCACGCGCTA | 32 |
| Bs2-Fwd | GCTTGCTTGTTTTTAATTCAGTTTATGGG | 33 |
| BS2-Rev | TTGAATGTCTGTGTTGGTGTGTAATCAC | 34 |
| BS3-Fwd | GTGAAAAAAAGGGGGTAGGCCCTC | 35 |
| BS3-Rev | CAGCCCAAAGTCAAAAGGCAAGATG | 36 |
| CTL-Fwd | GTTCCTTAACTGAGAGTTCCTCCTCCC | 37 |
| CTL-Rev | TCACTCTGGGCCGGAGTCAGTT | 38 |

1-45. (canceled)

46. A method of producing induced T-to-Natural-Killer [ITNK] cells from T cells and/or pro-T cells, the method comprising modulating the activity and/or effect of at least one Bcl11b gene and/or protein present in a T cell and/or pro-T cell, and converting said T cell and/or pro-T cell to an ITNK cell or cells.

47. A method of producing target T cells and/or target pro-T cells, the method comprising modulating the activity and/or effect of at least one BcI11b gene and/or protein product present in a T cell and/or pro-T cell, and converting said T cell and/or pro-T cell to said target T cells and/or target pro-T cells.

48. A method according to claim 46 wherein said modulating of the activity and/or effect of said Bcl11b gene and/or protein product comprises inhibiting said activity and/or effect.

49. A method according to claim 47 wherein said modulating of the activity and/or effect of said Bcl11b gene and/or protein product comprises inhibiting said activity and/or effect.

50. A method according to claim 46, wherein said inhibiting of the activity and/or effect of said Bcl11b gene and/or protein product comprises deletion of at least part of said Bcl11b gene.

51. A method according to claim 47, wherein said inhibiting of the activity and/or effect of said Bcl11b gene and/or protein product comprises deletion of at least part of said Bcl11b gene.

52. A method according to claim 46, wherein said modulating of the activity and/or effect of said Bcl11b gene and/or protein product comprises directly or indirectly modulating the activity and/or effect of said Bcl11b protein.

53. A method according to claim 47, wherein said modulating of the activity and/or effect of said Bcl11b gene and/or protein product comprises directly or indirectly modulating the activity and/or effect of said Bcl11b protein.

54. A method according to claim 46, which comprises directly or indirectly inhibiting the activity and/or effect of said Bcl11b protein.

55. A method according to claim 47, which comprises directly or indirectly inhibiting the activity and/or effect of said Bcl11b protein.

56. An isolated ITNK cell characterized by exhibiting one or more or all of the following properties:
(a) a morphology comparable to natural killer cells, in comparison to T cells;
(b) TCR β specific genomic DNA re-arrangement;
(c) a gene expression profile more similar to that of NK cells than the parental cells from which they were developed;
(d) cellular expression of one or more NK specific genes;
(e) decreased or no expression of one or more T lineage genes, in comparison to the parent cells from which the ITNK cell was derived;
(f) cell killing ability; and
(g) capable of recognizing MHC—I molecules and capable of killing MHC—I positive or negative cells when produced in vivo.

57. An isolated ITNK cell according to claim 56 obtainable, or obtained, from a T cell or pro-T cell.

58. An isolated ITNK cell obtained by carrying out a process as defined in claim 46.

59. An isolated target T cell or target pro-T cell including at least one Bcl11b gene product and/or protein product the activity and/or effect of which has been modulated compared to the corresponding gene and/or protein product in a precursor T cell or precursor pro-T cell, so that the target T cell or target pro-T cell is capable of converting to an ITNK cell.

60. An isolated target T cell or pro-T cell obtained by carrying out a process as defined in claim 47.

61. A method of treating a human or non-human mammal subject suffering from, or susceptible to disease such as cancer or viral infection, comprising administering to said subject a therapeutically effective amount of ITNK cells according to claims 54-56.

62. A method of treating a human or non-human mammal subject suffering from, or susceptible to disease such as cancer or viral infection, comprising administering to said subject a therapeutically effective amount of cells according to claim 57 or 58.

63. The method of claim 50 or 51, wherein said deletion comprises at least part of exon 4 of said Bcl11b gene.

64. The isolated ITNK cell of claim 56, wherein said cell is characterized by a gene expression profile more similar to that of LAK cells than the parental cells from which they were developed.

65. The isolated ITNK cell of claim 56, wherein said one or more specific NK genes are selected from the group consisting of ZFP105, IL2Rβ3, Id2, JAK1, NKG2D, NKG2A/C/E, B220, Rog (Zbtb32), Tnfrsfθ, Cdknic, Trail, Perforin, Interferon̂, NK1.1, NKp46, E4 bp4, NKG7, KLRD1, LTA, PLCG2, Ly49C/1 and Ly49G2.

66. The isolated ITNK cell of claim 56, wherein said one or more T lineage genes are selected from the group consisting of Notchi, Est1, Hes1, Gata3, Deltaxi, TCRβ, CD3, TcM 1 IL7R, T-bet and/or CD8a.

67. The isolated ITNK cell of claim 56, wherein said cell killing ability is characterized by the ability to prevent or ameliorate tumour formation or growth, the ability to kill stromal cells, tumour cells, or infected cells, in comparison to the precursor cell used (parent T cells or pro T cells).
